(19) United States
(12) Patent Application Publication
(10) Pub. No.: US 2022/0251051 A1
(43) Pub. Date: Aug. 11, 2022

(54) ORGANIC ELECTROLUMINESCENT COMPOUND AND ORGANIC ELECTROLUMINESCENT DEVICE THEREOF

(71) Applicants: ROHM AND HAAS ELECTRONIC MATERIALS KOREA LTD., Chungcheongnam-do (KR); ROHM AND HAAS ELECTRONIC MATERIALS SHANGHAI LTD., Shanghai (CN)

(72) Inventors: Chi-Sik KIM, Gyeonggi-do (KR); Soo-Yong LEE, Gyeonggi-do (KR); Dong Hyung LEE, Gyeonggi-do (KR); Yuchen LIU, Shanghai (CN)

(21) Appl. No.: 17/610,983

(22) PCT Filed: May 22, 2019
(86) PCT No.: PCT/CN2019/087896

§ 371 Date: Nov. 12, 2021

(51) Int. Cl. C07D 263/57; H01L 51/00; C07D 413/10; C07D 493/04; C07D 277/66; C07D 417/10; C07D 487/14; C07D 209/88; C07D 209/86; C07D 307/91; C07F 1/02; C07D 519/00

(52) U.S. Cl. CPC C07D 263/57; H01L 51/5016; C07D 413/10; C07D 493/04; C07D 277/66; C07D 417/10; C07D 487/14; C07D 209/88; C07D 209/86; C07D 307/91; C07F 1/02; H01L 51/0073; H01L 51/0074; H01L 51/0067; H01L 51/0072; C07D 519/00; H01L 51/0058

(57) ABSTRACT

The present disclosure relates to an organic electroluminescent compound and an organic electroluminescent device comprising the same. By comprising the organic electroluminescent compound of the present disclosure, it is possible to provide an organic electroluminescent device having an operating voltage lower than that of a conventional organic electroluminescent device and thus achieving higher power efficiency.

### TECHNICAL FIELD

The present disclosure relates to an organic electroluminescent compound and an organic electroluminescent device comprising the same.

### BACKGROUND ART

An electroluminescent (EL) device is a self-light-emitting device which has advantages in that it provides a wider viewing angle, a greater contrast ratio, and a faster response time. An organic EL device was first developed by Eastman Kodak in 1987, by using small aromatic diamine molecules and aluminum complexes as materials for forming a light-emitting layer [Appl. Phys. Lett. 51, 913, 1987].

In organic light emitting diodes (OLED), low operating voltage is important for increasing power efficiency. Specifically, the power efficiency of an OLED is given by [(π/voltage)×current efficiency], and thus the power efficiency is inversely proportional to the voltage. That is, the power efficiency can be increased by lowering an operating voltage of an OLED.

Meanwhile, Korean Patent Appl. Laid-Open No. 2017-0022865 (published on Mar. 2, 2017) discloses an organic electroluminescent device using a phenanthroxazole derivative as a red host. Also, Korean Patent Appl. Laid-Open No. 2017-0051198 (published on May 11, 2017) discloses an organic electroluminescent device using a phenanthroxazole derivative as an electron buffer layer or an electron transport layer. However, the above references do not specifically disclose an anthracenyl-containing compound.

### DISCLOSURE OF THE INVENTION

### Problems to be Solved

The objective of the present disclosure is to provide an organic electroluminescent compound effective to produce an organic electroluminescent device having an operating voltage lower than that of a conventional organic electroluminescent device and thus achieving higher power efficiency.

### Solution to Problems

Recently, in the field of an OLED, the red device and the green device have succeeded in lowering an operating voltage, but the blue device still has an operating voltage about 0.5 V to 1 V higher than that of the red device and the green device. Thus, there is a need for development to reduce an operating voltage of a blue organic electroluminescent device.

The present inventors have recognized that ETU (Electron Transfer Unit) is required for a blue host in order to reduce an operating voltage of a blue organic electroluminescent device. However, if the electron mobility increases, the lifespan of the blue layer gradually decreases. This is thought to be due to the increase of the electron attack to the adjacent layer such as HTL (Hole Transport Layer). As a result of studies to solve these problems, the present inventors have found that the above objective can be achieved by using a compound represented by the following formula 1, which has a phenanthrene fused with an ETU, as a blue host. Without wishing to be bound by theory, it is believed that a phenanthrene has a stronger resonance compared to a benzene or a naphthalene, so that the electrons can be more stabilized in a phenanthrene. It is also believed that a compound represented by the following formula 1 may have better electron mobility and good electron stability by increasing the resonance of ETU.

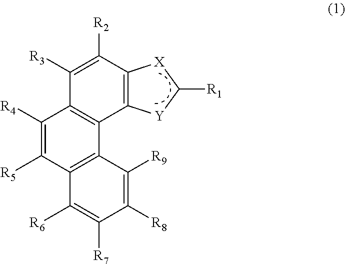

wherein

X represents —N═, —NR—, —O—, or —S—;

Y represents —N═, —NR—, —O—, or —S—; with the proviso that one of X and Y represents —N═, and the other of X and Y represents —NR—, —O—, or —S—;

R represents hydrogen, deuterium, a halogen, a cyano, a substituted or unsubstituted (C1-C30)alkyl, a substituted or unsubstituted (C6-C30)aryl, or a substituted or unsubstituted (5- to 30-membered)heteroaryl; and

R₁ to R₉, each independently, represent hydrogen, deuterium, a halogen, a cyano, a substituted or unsubstituted (C1-C30)alkyl, a substituted or unsubstituted (C6-C30)aryl, or a substituted or unsubstituted (5- to 30-membered)heteroaryl; with the proviso that at least one of R₁ to R9 represents a substituted or unsubstituted anthracenyl.

### Effects of the Invention

The organic electroluminescent device comprising the organic electroluminescent compound according to the present disclosure has an operating voltage lower than that of a conventional organic electroluminescent device, and thus can achieve higher power efficiency.

### EMBODIMENTS OF THE INVENTION

Hereinafter, the present disclosure will be described in detail. However, the following description is intended to explain the disclosure, and is not meant in any way to restrict the scope of the disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, an electron injection material, etc.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, iso-propyl, n-butyl, iso-butyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl” is meant to be a monocyclic or fused ring radical derived from an aromatic hydrocarbon having 6 to 30 ring backbone carbon atoms, in which the number of the ring backbone carbon atoms is preferably 6 to 25, more preferably 6 to 18. The above aryl may be partially saturated, and may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. More specifically, the aryl may include a phenyl group, a 1-naphthyl group, a 2-naphthyl group, a 1-anthryl group, a 2-anthryl group, a 9-anthryl group, a benzanthryl group, a 1-phenanthryl group, a 2-phenanthryl group, a 3-phenanthryl group, a 4-phenanthryl group, a 9-phenanthryl group, a naphthacenyl group, a pyrenyl group, a 1-chrysenyl group, a 2-chrysenyl group, a 3-chrysenyl group, a 4-chrysenyl group, a 5-chrysenyl group, a 6-chrysenyl group, a benzo[c]phenanthryl group, a benzo[g]chrysenyl group, a 1-triphenylenyl group, a 2-triphenylenyl group, a 3-triphenylenyl group, a 4-triphenylenyl group, a 1-fluorenyl group, a 2-fluorenyl group, a 3-fluorenyl group, a 4-fluorenyl group, a 9-fluorenyl group, a benzofluorenyl group, a dibenzofluorenyl group, a 2-biphenylyl group, a 3-biphenylyl group, a 4-biphenylyl group, an o-terphenyl group, an m-terphenyl-4-yl group, an m-terphenyl-3-yl group, an m-terphenyl-2-yl group, a p-terphenyl-4-yl group, a p-terphenyl-3-yl group, a p-terphenyl-2-yl group, an m-quaterphenyl group, a 3-fluoranthenyl group, a 4-fluoranthenyl group, an 8-fluoranthenyl group, a 9-fluoranthenyl group, a benzofluoranthenyl group, an o-tolyl group, an m-tolyl group, a p-tolyl group, a 2,3-xylyl group, a 3,4-xylyl group, a 2,5-xylyl group, a mesityl group, an o-cumenyl group, an m-cumenyl group, a p-cumenyl group, a p-t-butylphenyl group, a p-(2-phenylpropyl)phenyl group, a 4′-methylbiphenylyl group, a 4″-t-butyl-p-terphenyl-4-yl group, a 9,9-dimethyl-1-fluorenyl group, a 9,9-dimethyl-2-fluorenyl group, a 9,9-dimethyl-3-fluorenyl group, a 9,9-dimethyl-4-fluorenyl group, a 9,9-diphenyl-1-fluorenyl group, a 9,9-diphenyl-2-fluorenyl group, a 9,9-diphenyl-3-fluorenyl group, a 9,9-diphenyl-4-fluorenyl group, etc.

Herein, the term “(5- to 30-membered)heteroaryl” is meant to be an aryl group having 5 to 30 ring backbone atoms, and including at least one, preferably 1 to 4 heteroatoms selected from the group consisting of B, N, O, S, Si, and P. The above heteroaryl may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may include a 1-pyrrolyl group, a 2-pyrrolyl group, a 3-pyrrolyl group, a pyrazinyl group, a 2-pyridinyl group, a 2-pyrimidinyl group, a 4-pyrimidinyl group, a 5-pyrimidinyl group, a 6-pyrimidinyl group, a 1,2,3-triazin-4-yl group, a 1,2,4-triazin-3-yl group, a 1,3,5-triazin-2-yl group, a 1-imidazolyl group, a 2-imidazolyl group, a 1-pyrazolyl group, a 1-indolidinyl group, a 2-indolidinyl group, a 3-indolidinyl group, a 6-indolidinyl group, a 6-indolidinyl group, a 7-indolidinyl group, an 8-indolidinyl group, a 2-imidazopyndinyl group, a 3-imidazopyridinyl group, a 5-imidazopyridinyl group, a 6-imidazopyridinyl group, a 7-imidazopyridinyl group, an 8-imidazopyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a 2-quinolyl group, a 3-quinolyl group, a 4-quinolyl group, a 5-quinolyl group, a 6-quinolyl group, a 7-quinolyl group, an 8-quinolyl group, a 1-isoquinolyl group, a 3-isoquinolyl group, a 4-isoquinolyl group, a 5-isoquinolyl group, a 6-isoquinolyl group, a 7-isoquinolyl group, an 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, an azacarbazolyl-1-yl group, an azacarbazolyl-2-yl group, an azacarbazolyl-3-yl group, an azacarbazolyl-4-yl group, an azacarbazolyl-5-yl group, an azacarbazolyl-6-yl group, an azacarbazolyl-7-yl group, an azacarbazolyl-8-yl group, an azacarbazolyl-9-yl group, a 1-phenanthridinyl group, a 2-phenanthridinyl group, a 3-phenanthridinyl group, a 4-phenanthridinyl group, a 6-phenanthridinyl group, a 7-phenanthridinyl group, an 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrol-1-yl group, a 2-methylpyrrol-3-yl group, a 2-methylpyrrol-4-yl group, a 2-methylpyrrol-5-yl group, a 3-methylpyrrol-1-yl group, a 3-methylpyrrol-2-yl group, a 3-methylpyrrol-4-yl group, a 3-methylpyrrol-5-yl group, a 2-t-butylpyrrol-4-yl group, a 3-(2-phenylpropyl)pyrrol-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, a 4-t-butyl-3-indolyl group, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, a 1-silafluorenyl group, a 2-silafluorenyl group, a 3-silafluorenyl group, a 4-silafluorenyl group, a 1-germafluorenyl group, a 2-germafluorenyl group, a 3-germafluorenyl group, a 4-germafluorenyl group, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, In the present disclosure, the substituents of the substituted alkyl, the substituted aryl, the substituted heteroaryl, and the substituted anthracenyl, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (5- to 30-membered)heteroaryl unsubstituted or substituted with a (C6-C30)aryl; a (C6-C30)aryl unsubstituted or substituted with a (5- to 30-membered)heteroaryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl; a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of a (C1-C20)alkyl; a (C6-C25)aryl unsubstituted or substituted with a (C1-C20)alkyl(s) and/or a (5- to 25-membered)heteroaryl(s); and a (5- to 25-membered)heteroaryl unsubstituted or substituted with a (C6-C25)aryl(s). According to another embodiment of the present disclosure, the substituents, each independently, are at least one selected from the group consisting of a (C1-C10)alkyl; a (C6-C22)aryl unsubstituted or substituted with a (C1-C10)alkyl(s) and/or a (5- to 18-membered)heteroaryl(s); and a (5- to 20-membered)heteroaryl unsubstituted or substituted with a (C6-C18)aryl(s). For example, the substituents, each independently, may be at least one selected from the group consisting of a methyl, a phenyl, a naphthylphenyl, a phenyl substituted with a carbazolyl(s), a naphthyl, a phenylnaphthyl, a biphenylnaphthyl, a biphenyl, a dimethylfluorenyl, a phenanthrenyl unsubstituted or substituted with a phenyl(s), a terphenyl, a pyridyl substituted with a phenyl(s), a pyrimidinyl substituted with a phenyl(s), a benzofuranyl unsubstituted or substituted with a phenyl(s), a quinolyl substituted with a phenyl(s), a quinazolinyl substituted with a phenyl(s), a carbazolyl unsubstituted or substituted with a phenyl(s), a dibenzofuranyl, a dibenzothiophenyl, a benzofurobenzofuranyl, and a naphthobenzofuranyl.

Herein, the heteroaryl and the heterocycloalkyl, each independently, may contain at least one heteroatom selected from B, N, O, S, Si, and P. Also, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.
[symbol] to be bonded. For example, when X represents —N═, [symbol] bonded to X represents a double bond, and when X represents —O—, [symbol] bonded to X represents a single bond.

R represents hydrogen, deuterium, a halogen, a cyano, a substituted or unsubstituted (C1-C30)alkyl, a substituted or unsubstituted (C6-C30)aryl, or a substituted or unsubstituted (5- to 30-membered)heteroaryl.

In formula 1, R₁ to R₉, each independently, represent hydrogen, deuterium, a halogen, a cyano, a substituted or unsubstituted (C1-C30)alkyl, a substituted or unsubstituted (C6-C30)aryl, or a substituted or unsubstituted (5- to 30-membered)heteroaryl; with the proviso that at least one of R₁ to R₉ represents a substituted or unsubstituted anthracenyl. According to one embodiment of the present disclosure, R₁ to R₉, each independently, represent hydrogen, deuterium, or a substituted or unsubstituted (C6-C25)aryl; with the proviso that at least one of R₁ to R₉ represents a substituted anthracenyl. According to another embodiment of the present disclosure, R₁ represents a substituted or unsubstituted (C6-C18)aryl, and R₂ to R₉, each independently, represent hydrogen, deuterium, or a substituted or unsubstituted (C6-C22)aryl; with the proviso that at least one of R₁ to R₉ represents a substituted anthracenyl. For example, R₁ represents a phenyl or a substituted anthracenyl, and R₂ to R₉, each independently, represent hydrogen or a substituted anthracenyl; with the proviso that at least one of R₁ to R₉ represents a substituted anthracenyl. The substituent for the substituted anthracenyl, each independently, is at least one selected from the group consisting of a phenyl, a naphthylphenyl, a phenyl substituted with a carbazolyl(s), a naphthyl, a phenylnaphthyl, a biphenylnaphthyl, a biphenyl, a dimethylfluorenyl, a phenanthrenyl unsubstituted or substituted with a phenyl(s), a terphenyl, a pyridyl substituted with a phenyl(s), a pyrimidinyl substituted with a phenyl(s), a benzofuranyl unsubstituted or substituted with a phenyl(s), a quinolyl substituted with a phenyl(s), a quinazolinyl substituted with a phenyl(s), a carbazolyl unsubstituted or substituted with a phenyl(s), a dibenzofuranyl, a dibenzothiophenyl, a benzofurobenzofuranyl, and a naphthobenzofuranyl.

The formula 1 may be represented by any one of the following formulas 1-1 to 1-9.

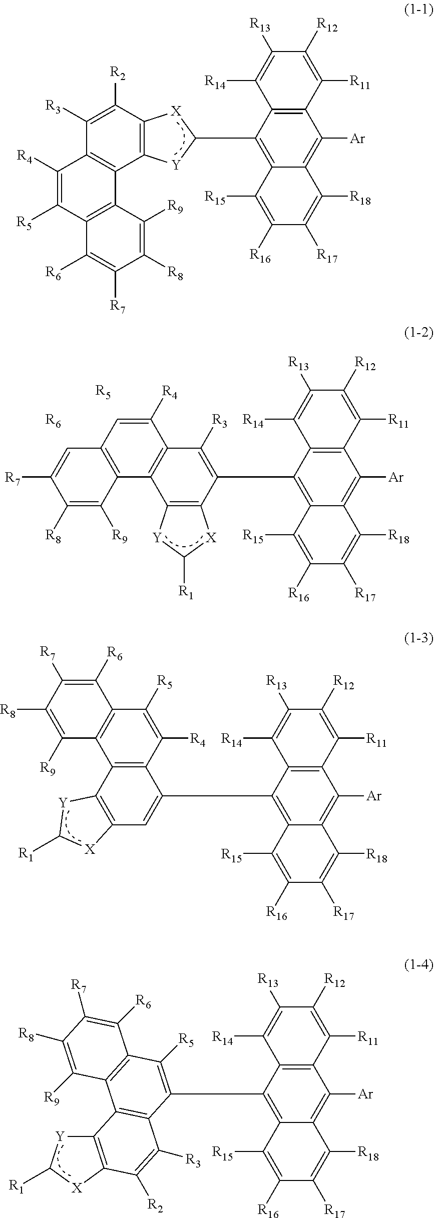

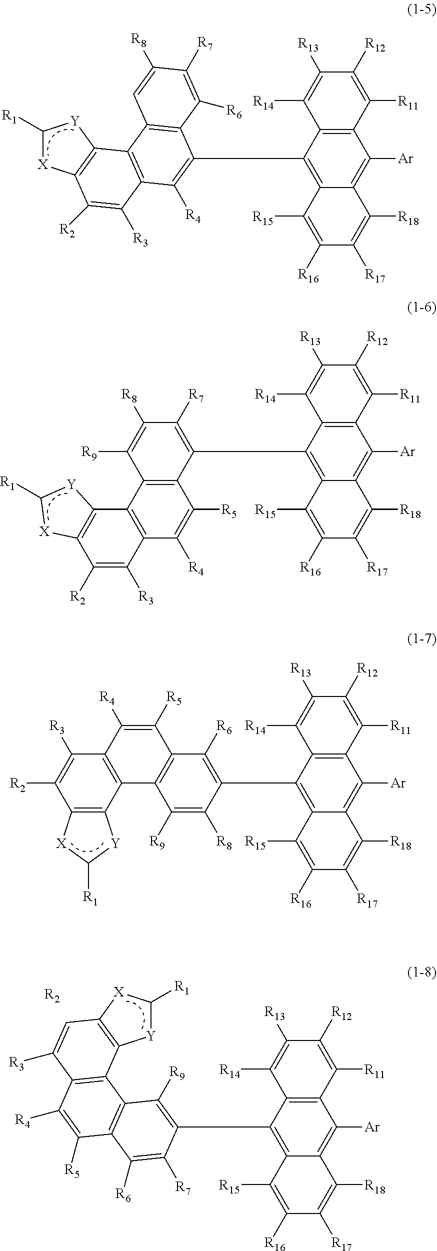

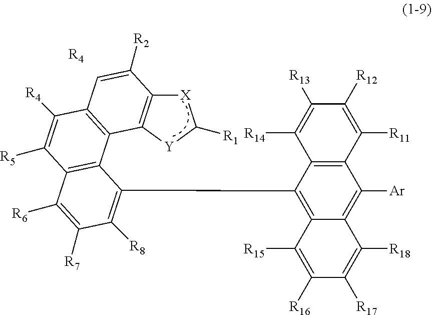

In formulas 1-1 to 1-9, R₁ to R₉, X, and Y are as defined in formula 1.

In formulas 1-1 to 1-9, R₁₁ to R₁₈, each independently, represent hydrogen, deuterium, a halogen, a cyano, a substituted or unsubstituted (C1-C30)alkyl, a substituted or unsubstituted (C6-C30)aryl, or a substituted or unsubstituted (5- to 30-membered)heteroaryl. For example, R₁₁ to R₁₅, each independently, represent hydrogen or deuterium.

In formulas 1-1 to 1-9, Ar represents a substituted or unsubstituted (C6-C30)aryl, or a substituted or unsubstituted (5- to 30-membered)heteroaryl. According to one embodiment of the present disclosure, Ar represents a substituted or unsubstituted (C6-C25)aryl, or a substituted or unsubstituted (5- to 20-membered)heteroaryl. According to another embodiment of the present disclosure, Ar represents a (C6-C25)aryl unsubstituted or substituted with at least one of a (C1-C10)alkyl(s), a (C6-C18)aryl(s) and a (5- to 20-membered)heteroaryl; or a (5- to 20-membered)heteroaryl unsubstituted or substituted with a (C6-C18)aryl(s). For example, Ar may represent a phenyl, a naphthylphenyl, a phenyl substituted with a carbazolyl(s), a naphthyl, a phenylnaphthyl, a biphenylnaphthyl, a biphenyl, a dimethylfluorenyl, a phenanthrenyl unsubstituted or substituted with a phenyl(s), a terphenyl, a pyridyl substituted with a phenyl(s), a pyrimidinyl substituted with a phenyl(s), a benzofuranyl unsubstituted or substituted with a phenyl(s), a quinolyl substituted with a phenyl(s), a quinazolinyl substituted with a phenyl(s), a carbazolyl unsubstituted or substituted with a phenyl(s), a dibenzofuranyl, a dibenzothiophenyl, a benzofurobenzofuranyl, or a naphthobenzofuranyl.

The compound represented by formula 1 may be selected from the group consisting of the following compounds, but is not limited thereto.

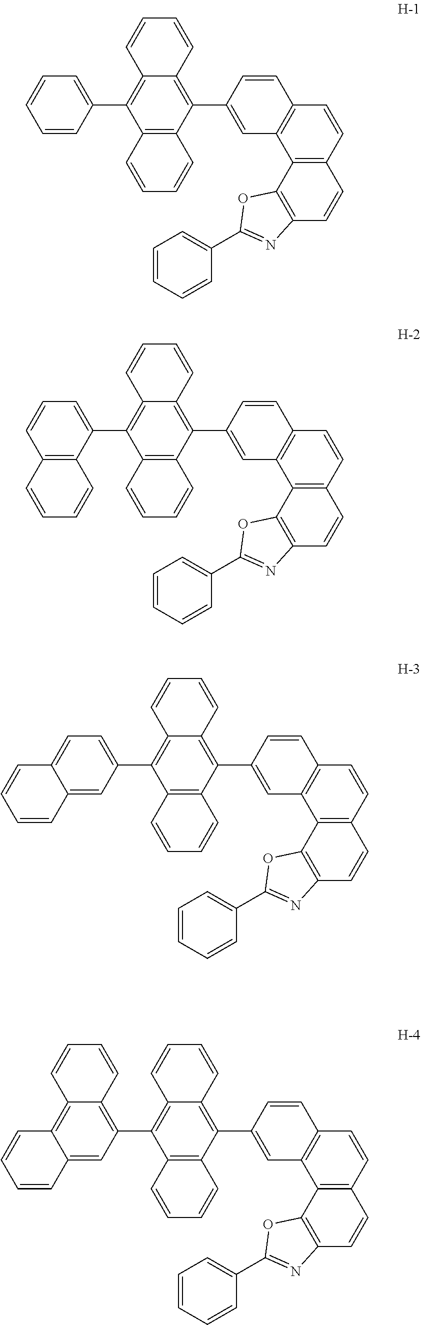

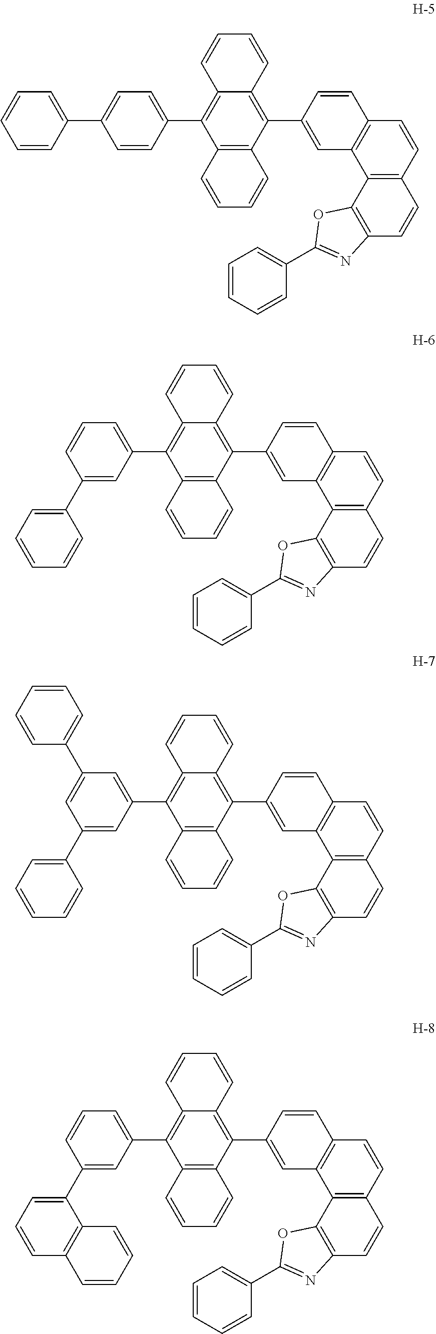 [SMILES: c1ccc(-c2ccc(-c3c4ccccc4c(-c4ccc5ccc6ccc7nc(-c8ccccc8)oc7c6c5c4)c4ccccc34)cc2)cc1 ; c1ccc(-c2cccc(-c3c4ccccc4c(-c4ccc5ccc6ccc7nc(-c8ccccc8)oc7c6c5c4)c4ccccc34)c2)cc1 ; c1ccc(-c2cc(-c3ccccc3)cc(-c3c4ccccc4c(-c4ccc5ccc6ccc7nc(-c8ccccc8)oc7c6c5c4)c4ccccc34)c2)cc1 ; c1ccc(-c2nc3ccc4ccc5ccc(-c6c7ccccc7c(-c7cccc(-c8cccc9ccccc89)c7)c7ccccc67)cc5c4c3o2)cc1]

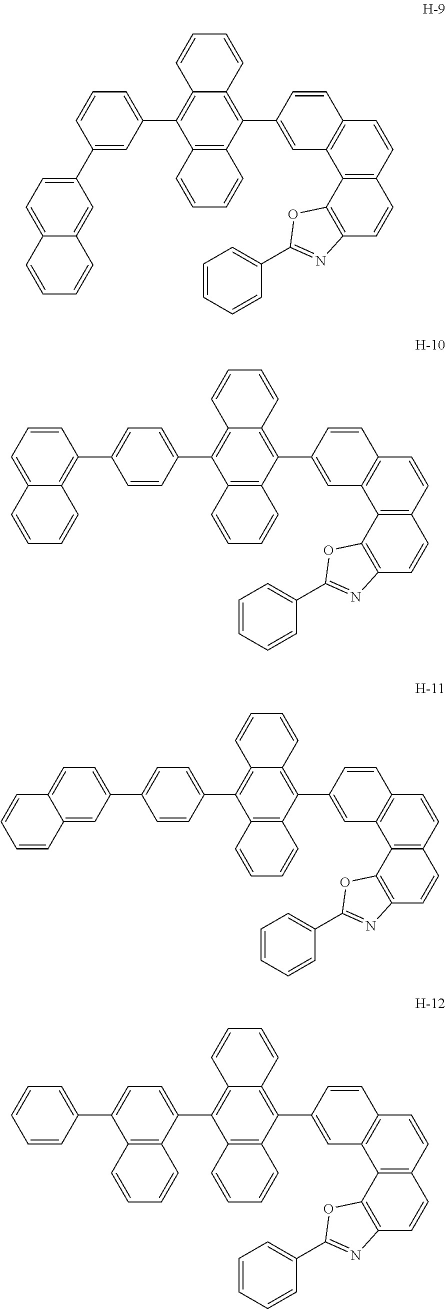 [SMILES: c1ccc(-c2nc3ccc4ccc5ccc(-c6c7ccccc7c(-c7cccc(-c8ccc9ccccc9c8)c7)c7ccccc67)cc5c4c3o2)cc1 ; c1ccc(-c2nc3ccc4ccc5ccc(-c6c7ccccc7c(-c7ccc(-c8cccc9ccccc89)cc7)c7ccccc67)cc5c4c3o2)cc1 ; c1ccc(-c2nc3ccc4ccc5ccc(-c6c7ccccc7c(-c7ccc(-c8ccc9ccccc9c8)cc7)c7ccccc67)cc5c4c3o2)cc1 ; c1ccc(-c2nc3ccc4ccc5ccc(-c6c7ccccc7c(-c7ccc(-c8ccccc8)c8ccccc78)c7ccccc67)cc5c4c3o2)cc1]

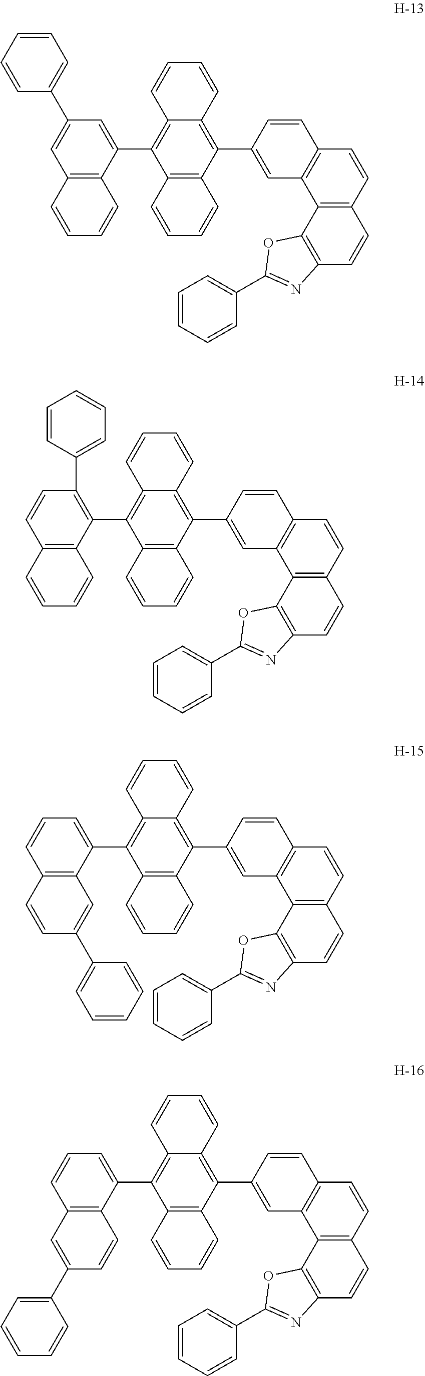 [SMILES: c1ccc(-c2cc(-c3c4ccccc4c(-c4ccc5ccc6ccc7nc(-c8ccccc8)oc7c6c5c4)c4ccccc34)c3ccccc3c2)cc1 ; c1ccc(-c2nc3ccc4ccc5ccc(-c6c7ccccc7c(-c7c(-c8ccccc8)ccc8ccccc78)c7ccccc67)cc5c4c3o2)cc1 ; c1ccc(-c2ccc3cccc(-c4c5ccccc5c(-c5ccc6ccc7ccc8nc(-c9ccccc9)oc8c7c6c5)c5ccccc45)c3c2)cc1 ; c1ccc(-c2ccc3c(-c4c5ccccc5c(-c5ccc6ccc7ccc8nc(-c9ccccc9)oc8c7c6c5)c5ccccc45)cccc3c2)cc1]

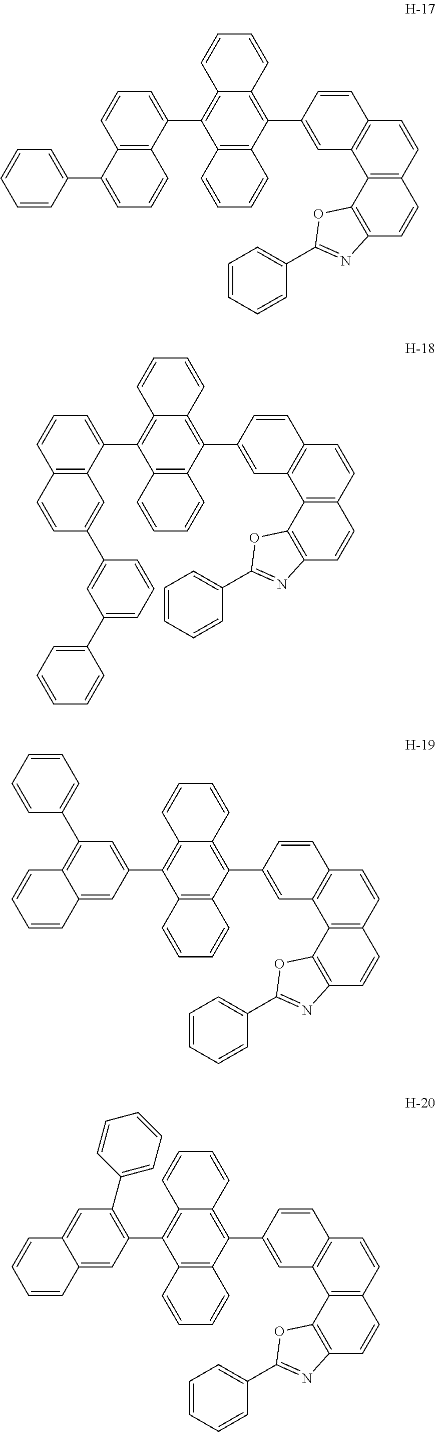 [SMILES: c1ccc(-c2nc3ccc4ccc5ccc(-c6c7ccccc7c(-c7cccc8c(-c9ccccc9)cccc78)c7ccccc67)cc5c4c3o2)cc1 ; c1ccc(-c2cccc(-c3ccc4cccc(-c5c6ccccc6c(-c6ccc7ccc8ccc9nc(-c%10ccccc%10)oc9c8c7c6)c6ccccc56)c4c3)c2)cc1 ; c1ccc(-c2nc3ccc4ccc5ccc(-c6c7ccccc7c(-c7cc(-c8ccccc8)c8ccccc8c7)c7ccccc67)cc5c4c3o2)cc1 ; c1ccc(-c2nc3ccc4ccc5ccc(-c6c7ccccc7c(-c7cc8ccccc8cc7-c7ccccc7)c7ccccc67)cc5c4c3o2)cc1]

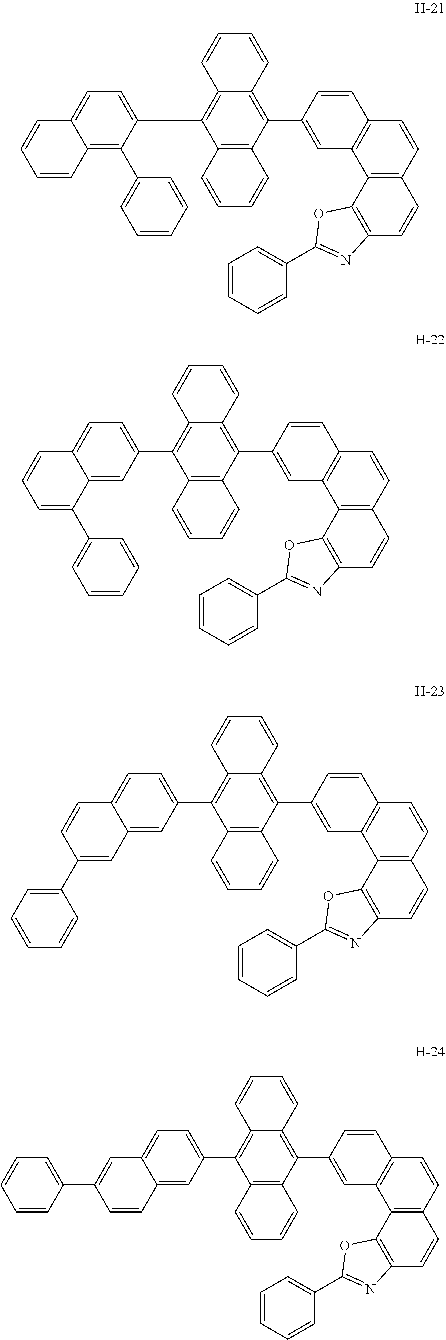 [SMILES: c1ccc(-c2nc3ccc4ccc5ccc(-c6c7ccccc7c(-c7ccc8ccccc8c7-c7ccccc7)c7ccccc67)cc5c4c3o2)cc1 ; c1ccc(-c2nc3ccc4ccc5ccc(-c6c7ccccc7c(-c7ccc8cccc(-c9ccccc9)c8c7)c7ccccc67)cc5c4c3o2)cc1 ; c1ccc(-c2ccc3ccc(-c4c5ccccc5c(-c5ccc6ccc7ccc8nc(-c9ccccc9)oc8c7c6c5)c5ccccc45)cc3c2)cc1 ; c1ccc(-c2ccc3cc(-c4c5ccccc5c(-c5ccc6ccc7ccc8nc(-c9ccccc9)oc8c7c6c5)c5ccccc45)ccc3c2)cc1]

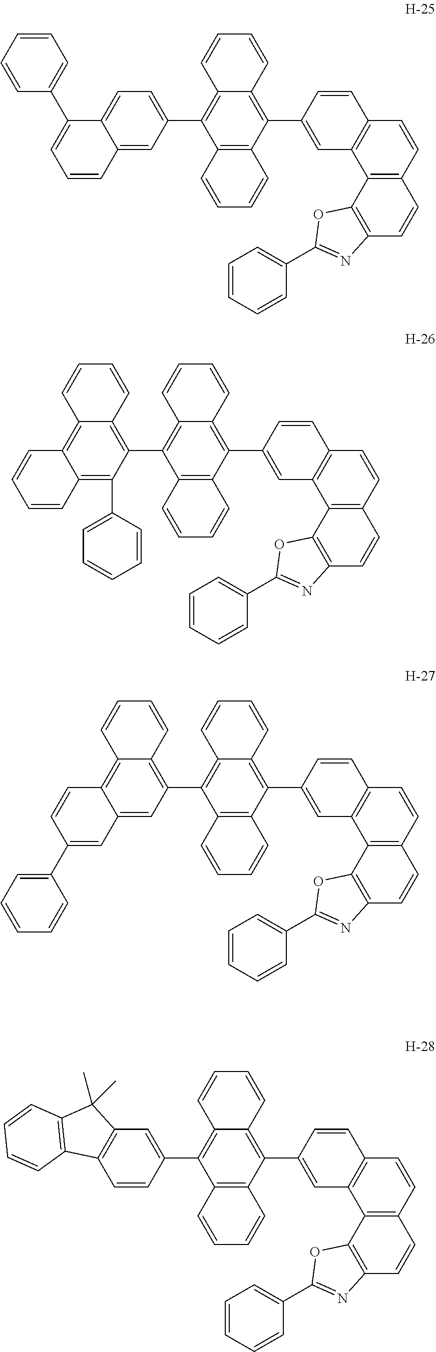 [SMILES: c1ccc(-c2nc3ccc4ccc5ccc(-c6c7ccccc7c(-c7ccc8c(-c9ccccc9)cccc8c7)c7ccccc67)cc5c4c3o2)cc1 ; c1ccc(-c2nc3ccc4ccc5ccc(-c6c7ccccc7c(-c7c(-c8ccccc8)c8ccccc8c8ccccc78)c7ccccc67)cc5c4c3o2)cc1 ; c1ccc(-c2ccc3c(c2)cc(-c2c4ccccc4c(-c4ccc5ccc6ccc7nc(-c8ccccc8)oc7c6c5c4)c4ccccc24)c2ccccc23)cc1 ; CC1(C)c2ccccc2-c2ccc(-c3c4ccccc4c(-c4ccc5ccc6ccc7nc(-c8ccccc8)oc7c6c5c4)c4ccccc34)cc21]

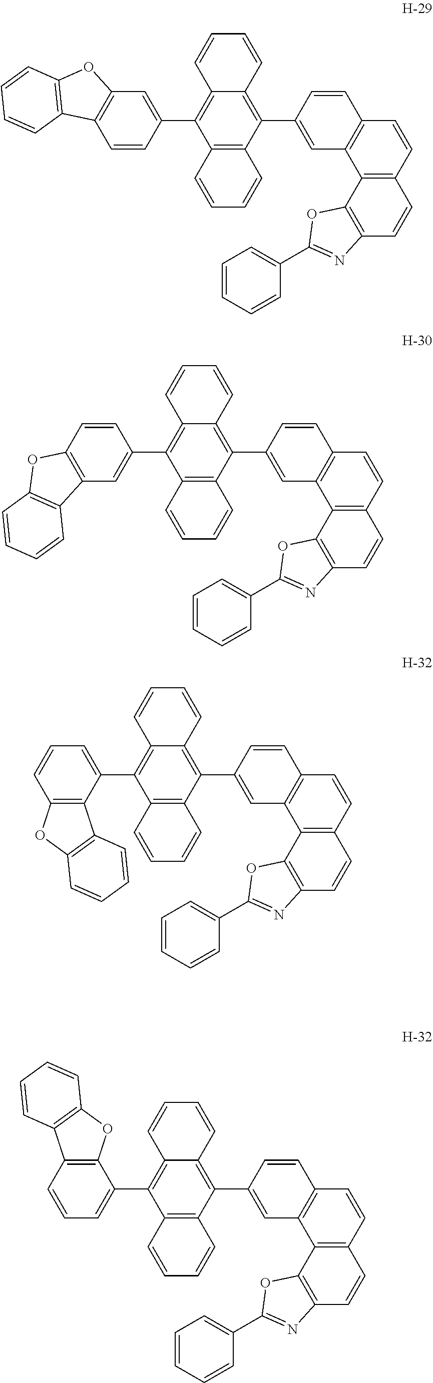 [SMILES: c1ccc(-c2nc3ccc4ccc5ccc(-c6c7ccccc7c(-c7ccc8c(c7)oc7ccccc78)c7ccccc67)cc5c4c3o2)cc1 ; c1ccc(-c2nc3ccc4ccc5ccc(-c6c7ccccc7c(-c7ccc8oc9ccccc9c8c7)c7ccccc67)cc5c4c3o2)cc1 ; c1ccc(-c2nc3ccc4ccc5ccc(-c6c7ccccc7c(-c7cccc8oc9ccccc9c78)c7ccccc67)cc5c4c3o2)cc1 ; c1ccc(-c2nc3ccc4ccc5ccc(-c6c7ccccc7c(-c7cccc8c7oc7ccccc78)c7ccccc67)cc5c4c3o2)cc1]

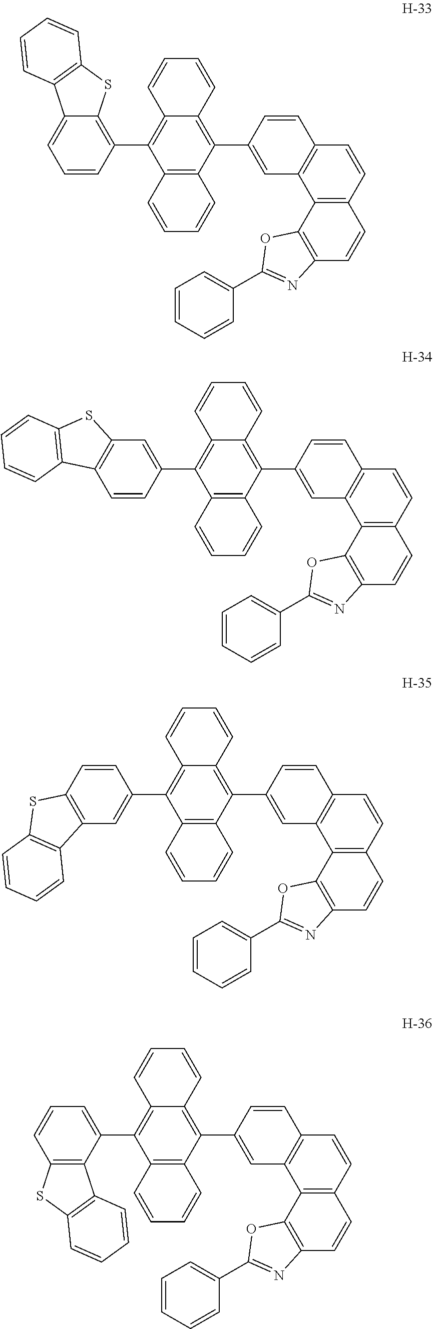

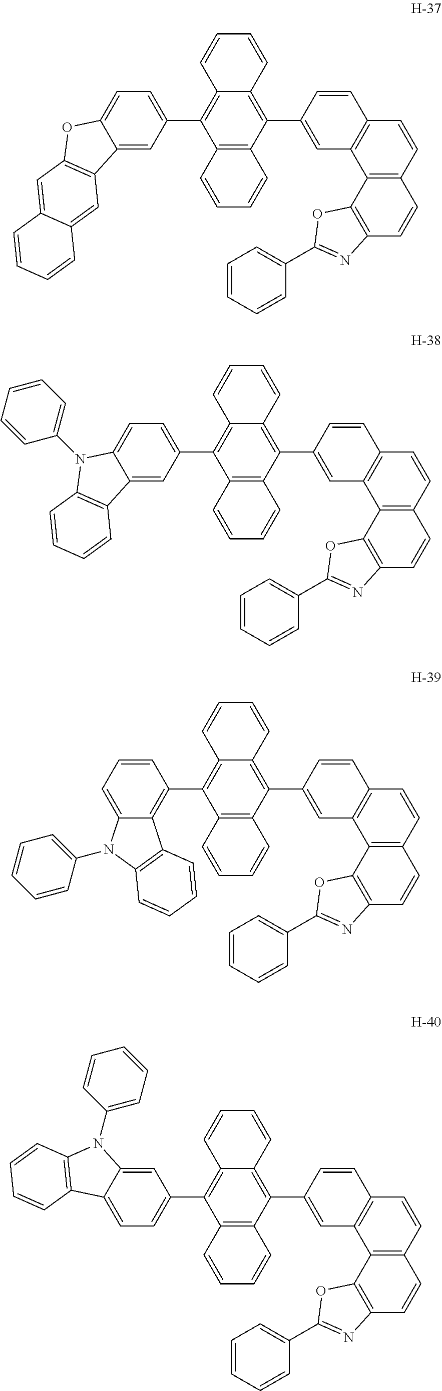 [SMILES: c1ccc(-c2nc3ccc4ccc5ccc(-c6c7ccccc7c(-c7ccc8oc9cc%10ccccc%10cc9c8c7)c7ccccc67)cc5c4c3o2)cc1 ; c1ccc(-c2nc3ccc4ccc5ccc(-c6c7ccccc7c(-c7ccc8c(c7)c7ccccc7n8-c7ccccc7)c7ccccc67)cc5c4c3o2)cc1 ; c1ccc(-c2nc3ccc4ccc5ccc(-c6c7ccccc7c(-c7cccc8c7c7ccccc7n8-c7ccccc7)c7ccccc67)cc5c4c3o2)cc1 ; c1ccc(-c2nc3ccc4ccc5ccc(-c6c7ccccc7c(-c7ccc8c9ccccc9n(-c9ccccc9)c8c7)c7ccccc67)cc5c4c3o2)cc1]

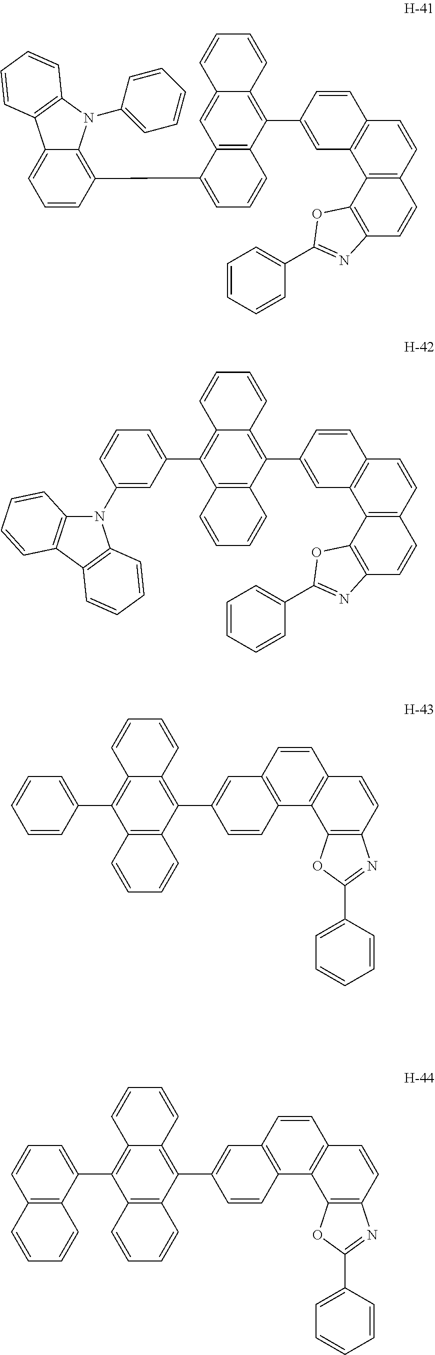 [SMILES: c1ccc(-c2nc3ccc4ccc5ccc(-c6c7ccccc7cc7c(-c8cccc9c%10ccccc%10n(-c%10ccccc%10)c89)cccc67)cc5c4c3o2)cc1 ; c1ccc(-c2nc3ccc4ccc5ccc(-c6c7ccccc7c(-c7cccc(-n8c9ccccc9c9ccccc98)c7)c7ccccc67)cc5c4c3o2)cc1 ; c1ccc(-c2nc3ccc4ccc5cc(-c6c7ccccc7c(-c7ccccc7)c7ccccc67)ccc5c4c3o2)cc1 ; c1ccc(-c2nc3ccc4ccc5cc(-c6c7ccccc7c(-c7cccc8ccccc78)c7ccccc67)ccc5c4c3o2)cc1]

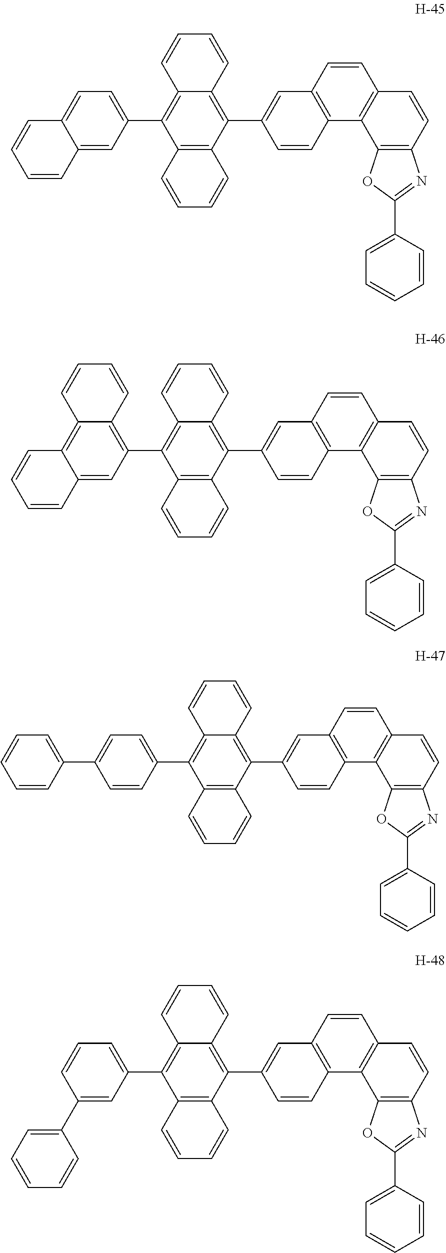 [SMILES: c1ccc(-c2nc3ccc4ccc5cc(-c6c7ccccc7c(-c7ccc8ccccc8c7)c7ccccc67)ccc5c4c3o2)cc1 ; c1ccc(-c2nc3ccc4ccc5cc(-c6c7ccccc7c(-c7cc8ccccc8c8ccccc78)c7ccccc67)ccc5c4c3o2)cc1 ; c1ccc(-c2ccc(-c3c4ccccc4c(-c4ccc5c(ccc6ccc7nc(-c8ccccc8)oc7c65)c4)c4ccccc34)cc2)cc1 ; c1ccc(-c2cccc(-c3c4ccccc4c(-c4ccc5c(ccc6ccc7nc(-c8ccccc8)oc7c65)c4)c4ccccc34)c2)cc1]

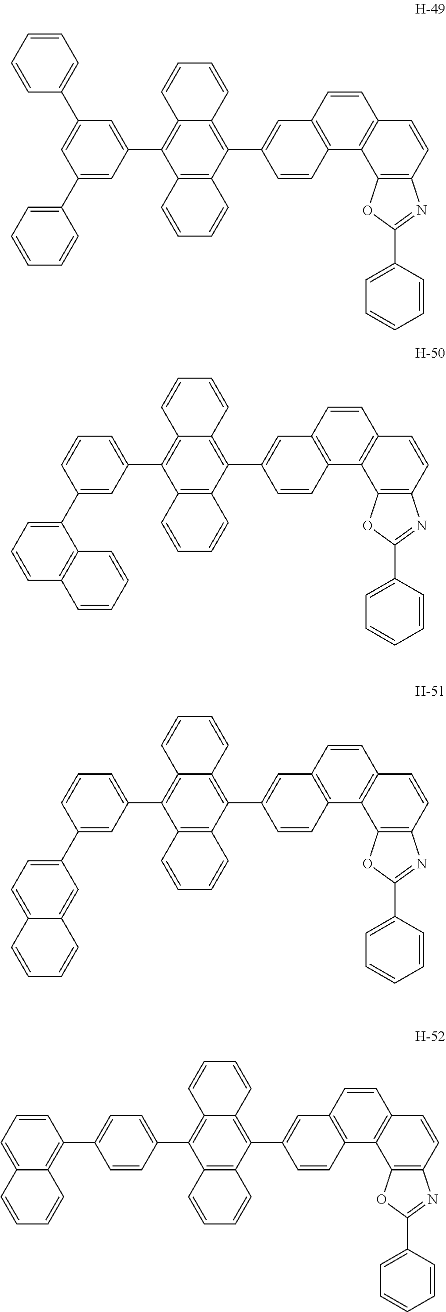 [SMILES: c1ccc(-c2cc(-c3ccccc3)cc(-c3c4ccccc4c(-c4ccc5c(ccc6ccc7nc(-c8ccccc8)oc7c65)c4)c4ccccc34)c2)cc1 ; c1ccc(-c2nc3ccc4ccc5cc(-c6c7ccccc7c(-c7cccc(-c8cccc9ccccc89)c7)c7ccccc67)ccc5c4c3o2)cc1 ; c1ccc(-c2nc3ccc4ccc5cc(-c6c7ccccc7c(-c7cccc(-c8ccc9ccccc9c8)c7)c7ccccc67)ccc5c4c3o2)cc1 ; c1ccc(-c2nc3ccc4ccc5cc(-c6c7ccccc7c(-c7ccc(-c8cccc9ccccc89)cc7)c7ccccc67)ccc5c4c3o2)cc1]

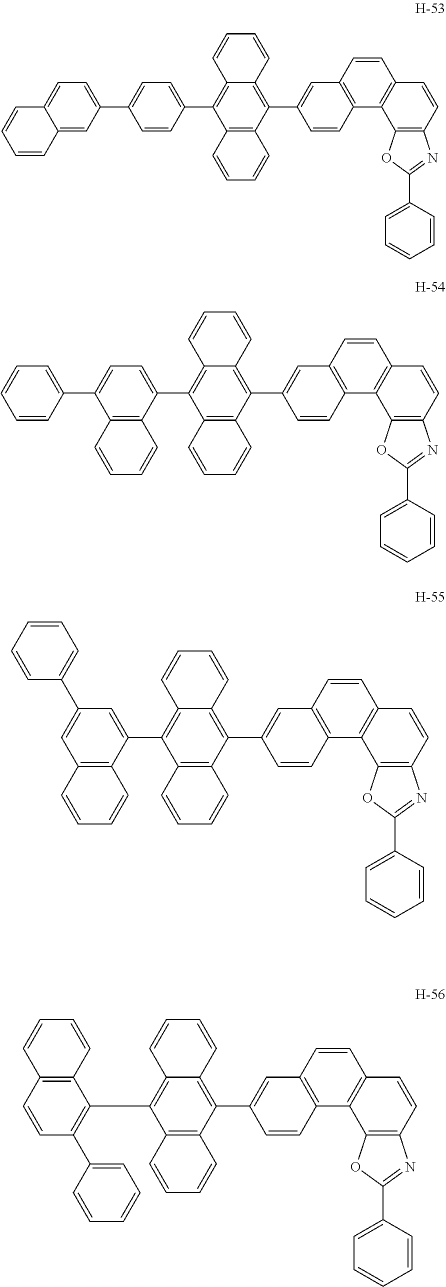 [SMILES: c1ccc(-c2nc3ccc4ccc5cc(-c6c7ccccc7c(-c7ccc(-c8ccc9ccccc9c8)cc7)c7ccccc67)ccc5c4c3o2)cc1 ; c1ccc(-c2nc3ccc4ccc5cc(-c6c7ccccc7c(-c7ccc(-c8ccccc8)c8ccccc78)c7ccccc67)ccc5c4c3o2)cc1 ; c1ccc(-c2cc(-c3c4ccccc4c(-c4ccc5c(ccc6ccc7nc(-c8ccccc8)oc7c65)c4)c4ccccc34)c3ccccc3c2)cc1 ; c1ccc(-c2nc3ccc4ccc5cc(-c6c7ccccc7c(-c7c(-c8ccccc8)ccc8ccccc78)c7ccccc67)ccc5c4c3o2)cc1]

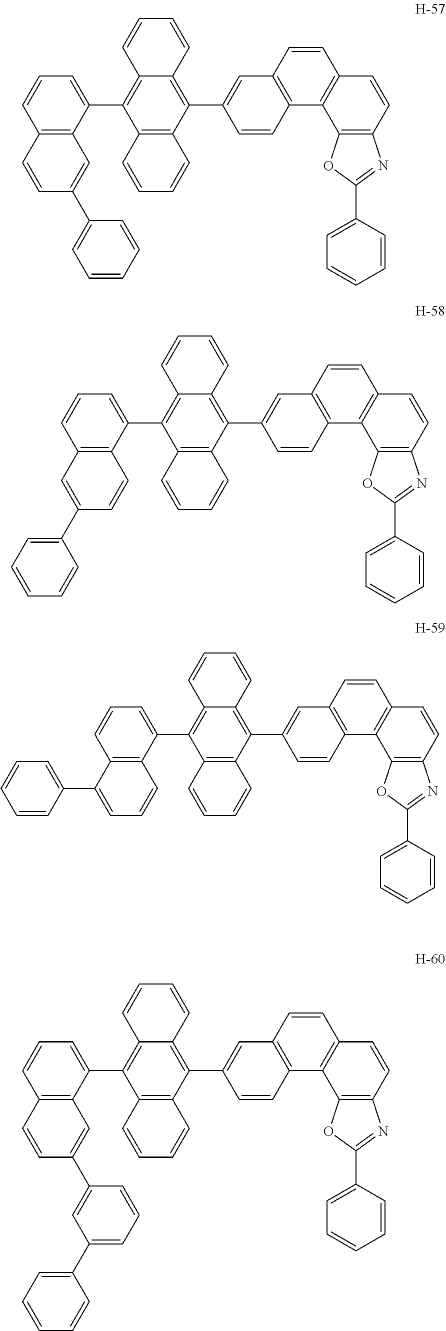 [SMILES: c1ccc(-c2ccc3cccc(-c4c5ccccc5c(-c5ccc6c(ccc7ccc8nc(-c9ccccc9)oc8c76)c5)c5ccccc45)c3c2)cc1 ; c1ccc(-c2ccc3c(-c4c5ccccc5c(-c5ccc6c(ccc7ccc8nc(-c9ccccc9)oc8c76)c5)c5ccccc45)cccc3c2)cc1 ; c1ccc(-c2nc3ccc4ccc5cc(-c6c7ccccc7c(-c7cccc8c(-c9ccccc9)cccc78)c7ccccc67)ccc5c4c3o2)cc1 ; c1ccc(-c2cccc(-c3ccc4cccc(-c5c6ccccc6c(-c6ccc7c(ccc8ccc9nc(-c%10ccccc%10)oc9c87)c6)c6ccccc56)c4c3)c2)cc1]

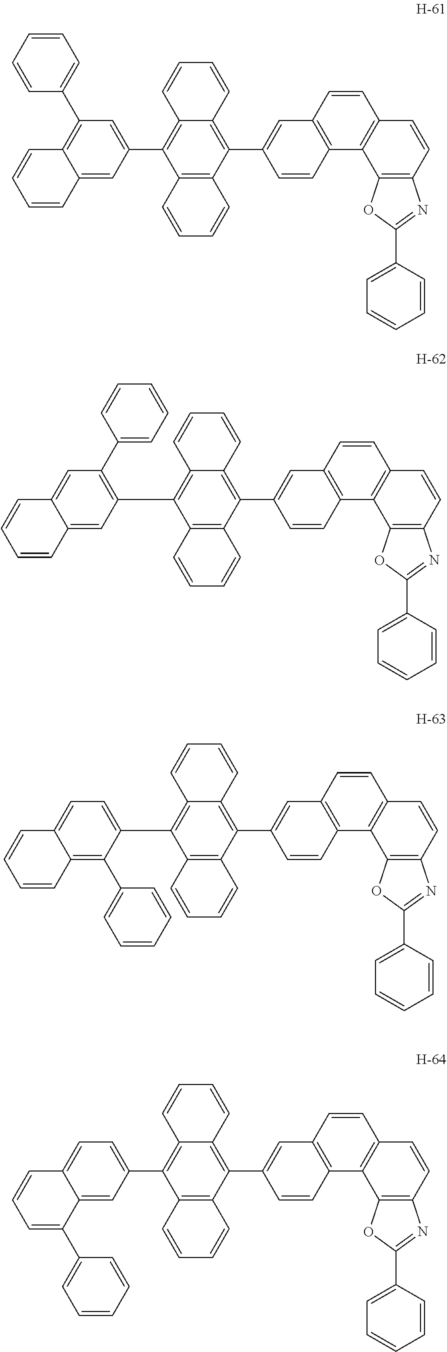 [SMILES: c1ccc(-c2nc3ccc4ccc5cc(-c6c7ccccc7c(-c7cc(-c8ccccc8)c8ccccc8c7)c7ccccc67)ccc5c4c3o2)cc1 ; c1ccc(-c2nc3ccc4ccc5cc(-c6c7ccccc7c(-c7cc8ccccc8cc7-c7ccccc7)c7ccccc67)ccc5c4c3o2)cc1 ; c1ccc(-c2nc3ccc4ccc5cc(-c6c7ccccc7c(-c7ccc8ccccc8c7-c7ccccc7)c7ccccc67)ccc5c4c3o2)cc1 ; c1ccc(-c2nc3ccc4ccc5cc(-c6c7ccccc7c(-c7ccc8cccc(-c9ccccc9)c8c7)c7ccccc67)ccc5c4c3o2)cc1]

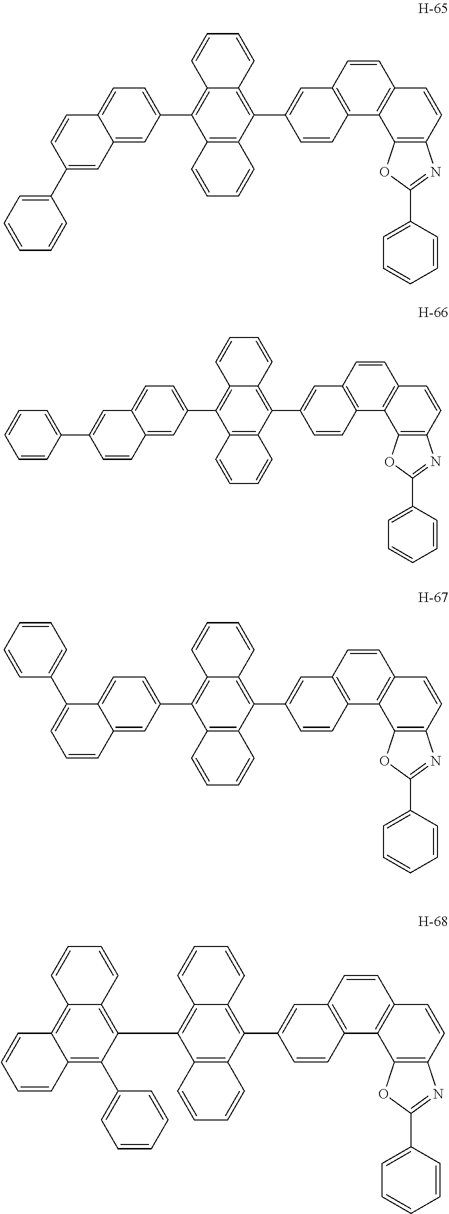 [SMILES: c1ccc(-c2ccc3ccc(-c4c5ccccc5c(-c5ccc6c(ccc7ccc8nc(-c9ccccc9)oc8c76)c5)c5ccccc45)cc3c2)cc1 ; c1ccc(-c2ccc3cc(-c4c5ccccc5c(-c5ccc6c(ccc7ccc8nc(-c9ccccc9)oc8c76)c5)c5ccccc45)ccc3c2)cc1 ; c1ccc(-c2nc3ccc4ccc5cc(-c6c7ccccc7c(-c7ccc8c(-c9ccccc9)cccc8c7)c7ccccc67)ccc5c4c3o2)cc1 ; c1ccc(-c2nc3ccc4ccc5cc(-c6c7ccccc7c(-c7c(-c8ccccc8)c8ccccc8c8ccccc78)c7ccccc67)ccc5c4c3o2)cc1]

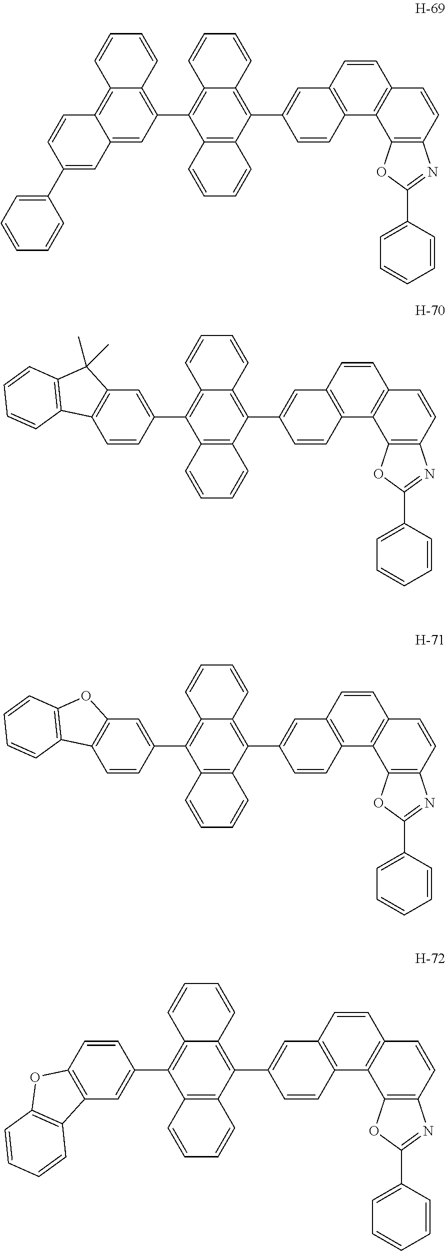 [SMILES: c1ccc(-c2ccc3c(c2)cc(-c2c4ccccc4c(-c4ccc5c(ccc6ccc7nc(-c8ccccc8)oc7c65)c4)c4ccccc24)c2ccccc23)cc1 ; CC1(C)c2ccccc2-c2ccc(-c3c4ccccc4c(-c4ccc5c(ccc6ccc7nc(-c8ccccc8)oc7c65)c4)c4ccccc34)cc21 ; c1ccc(-c2nc3ccc4ccc5cc(-c6c7ccccc7c(-c7ccc8c(c7)oc7ccccc78)c7ccccc67)ccc5c4c3o2)cc1 ; c1ccc(-c2nc3ccc4ccc5cc(-c6c7ccccc7c(-c7ccc8oc9ccccc9c8c7)c7ccccc67)ccc5c4c3o2)cc1]

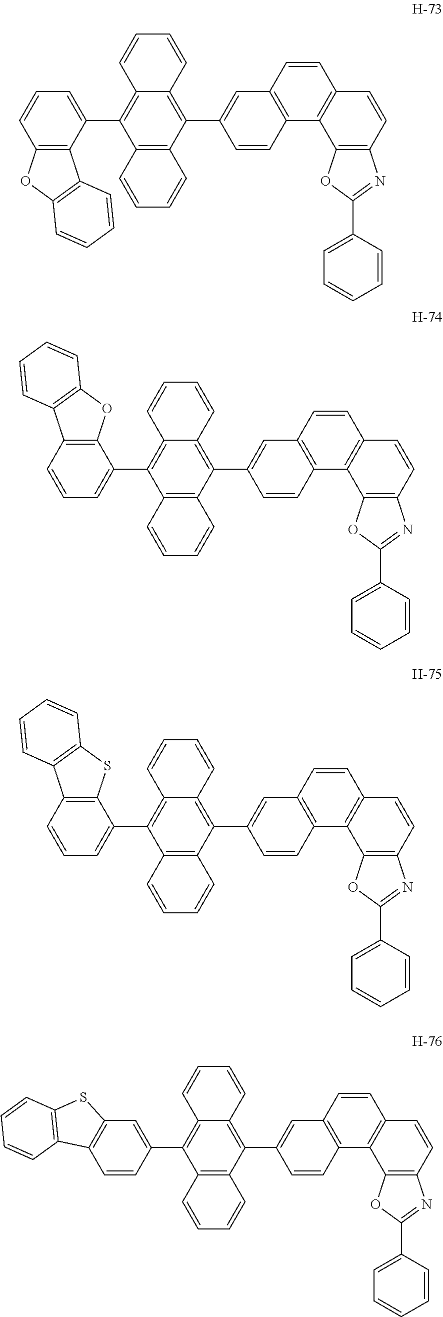 [SMILES: c1ccc(-c2nc3ccc4ccc5cc(-c6c7ccccc7c(-c7cccc8oc9ccccc9c78)c7ccccc67)ccc5c4c3o2)cc1 ; c1ccc(-c2nc3ccc4ccc5cc(-c6c7ccccc7c(-c7cccc8c7oc7ccccc78)c7ccccc67)ccc5c4c3o2)cc1 ; c1ccc(-c2nc3ccc4ccc5cc(-c6c7ccccc7c(-c7cccc8c7sc7ccccc78)c7ccccc67)ccc5c4c3o2)cc1 ; c1ccc(-c2nc3ccc4ccc5cc(-c6c7ccccc7c(-c7ccc8c(c7)sc7ccccc78)c7ccccc67)ccc5c4c3o2)cc1]

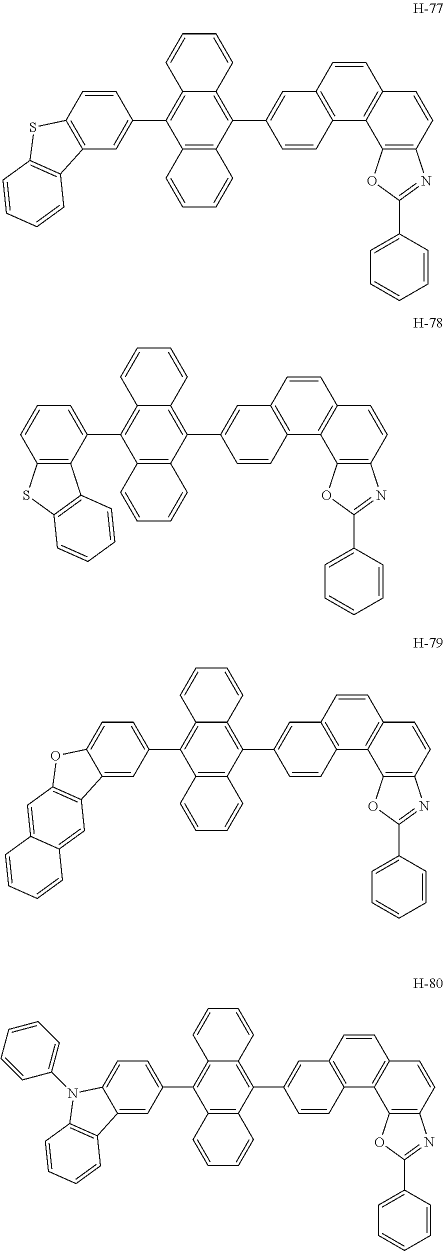 [SMILES: c1ccc(-c2nc3ccc4ccc5cc(-c6c7ccccc7c(-c7ccc8sc9ccccc9c8c7)c7ccccc67)ccc5c4c3o2)cc1 ; c1ccc(-c2nc3ccc4ccc5cc(-c6c7ccccc7c(-c7cccc8sc9ccccc9c78)c7ccccc67)ccc5c4c3o2)cc1 ; c1ccc(-c2nc3ccc4ccc5cc(-c6c7ccccc7c(-c7ccc8oc9cc%10ccccc%10cc9c8c7)c7ccccc67)ccc5c4c3o2)cc1 ; c1ccc(-c2nc3ccc4ccc5cc(-c6c7ccccc7c(-c7ccc8c(c7)c7ccccc7n8-c7ccccc7)c7ccccc67)ccc5c4c3o2)cc1]

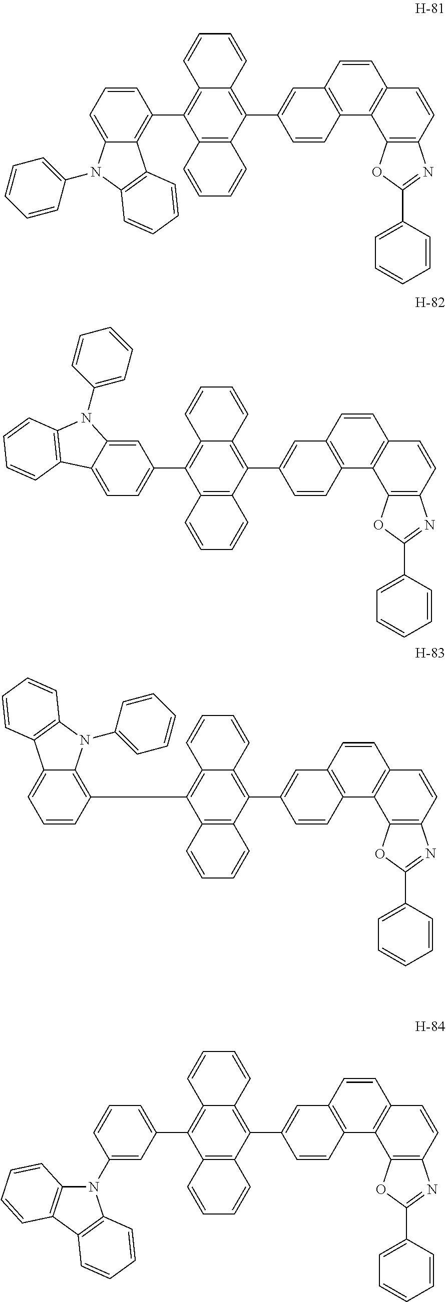 [SMILES: c1ccc(-c2nc3ccc4ccc5cc(-c6c7ccccc7c(-c7cccc8c7c7ccccc7n8-c7ccccc7)c7ccccc67)ccc5c4c3o2)cc1 ; c1ccc(-c2nc3ccc4ccc5cc(-c6c7ccccc7c(-c7ccc8c9ccccc9n(-c9ccccc9)c8c7)c7ccccc67)ccc5c4c3o2)cc1 ; c1ccc(-c2nc3ccc4ccc5cc(-c6c7ccccc7c(-c7cccc8c9ccccc9n(-c9ccccc9)c78)c7ccccc67)ccc5c4c3o2)cc1 ; c1ccc(-c2nc3ccc4ccc5cc(-c6c7ccccc7c(-c7cccc(-n8c9ccccc9c9ccccc98)c7)c7ccccc67)ccc5c4c3o2)cc1]

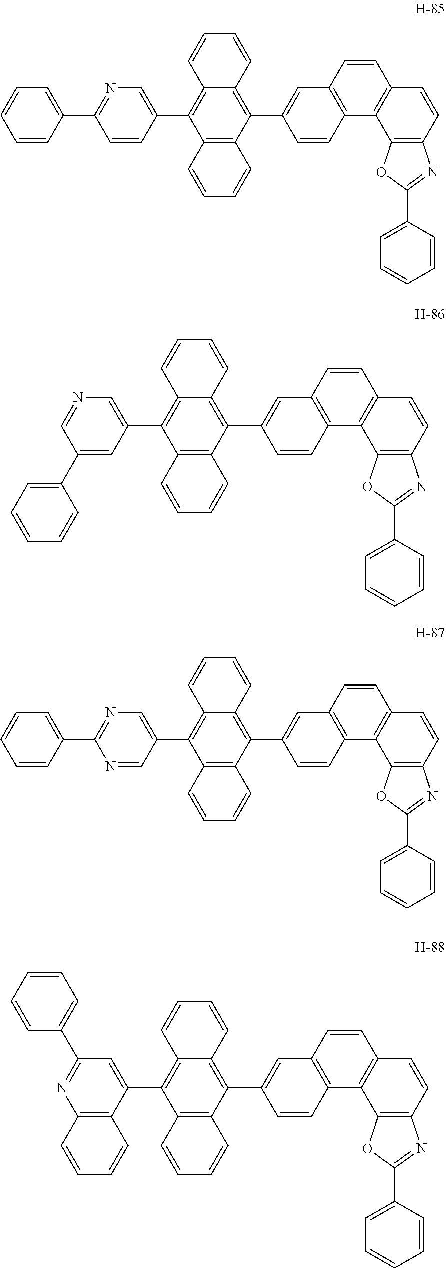 [SMILES: c1ccc(-c2ccc(-c3c4ccccc4c(-c4ccc5c(ccc6ccc7nc(-c8ccccc8)oc7c65)c4)c4ccccc34)cn2)cc1 ; c1ccc(-c2cncc(-c3c4ccccc4c(-c4ccc5c(ccc6ccc7nc(-c8ccccc8)oc7c65)c4)c4ccccc34)c2)cc1 ; c1ccc(-c2ncc(-c3c4ccccc4c(-c4ccc5c(ccc6ccc7nc(-c8ccccc8)oc7c65)c4)c4ccccc34)cn2)cc1 ; c1ccc(-c2cc(-c3c4ccccc4c(-c4ccc5c(ccc6ccc7nc(-c8ccccc8)oc7c65)c4)c4ccccc34)c3ccccc3n2)cc1]

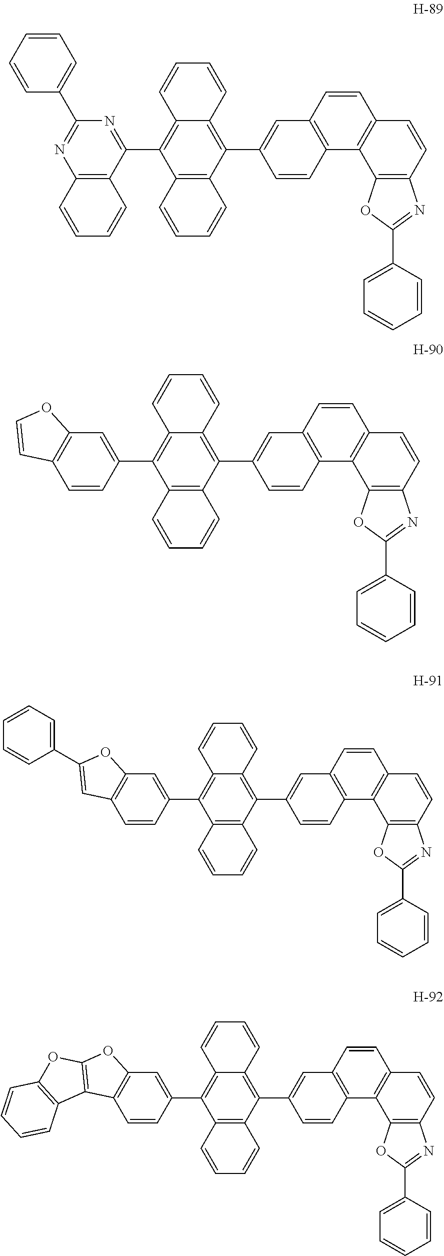 [SMILES: c1ccc(-c2nc(-c3c4ccccc4c(-c4ccc5c(ccc6ccc7nc(-c8ccccc8)oc7c65)c4)c4ccccc34)c3ccccc3n2)cc1 ; c1ccc(-c2nc3ccc4ccc5cc(-c6c7ccccc7c(-c7ccc8ccoc8c7)c7ccccc67)ccc5c4c3o2)cc1 ; c1ccc(-c2cc3ccc(-c4c5ccccc5c(-c5ccc6c(ccc7ccc8nc(-c9ccccc9)oc8c76)c5)c5ccccc45)cc3o2)cc1 ; c1ccc(-c2nc3ccc4ccc5cc(-c6c7ccccc7c(-c7ccc8c(c7)oc7oc9ccccc9c78)c7ccccc67)ccc5c4c3o2)cc1]

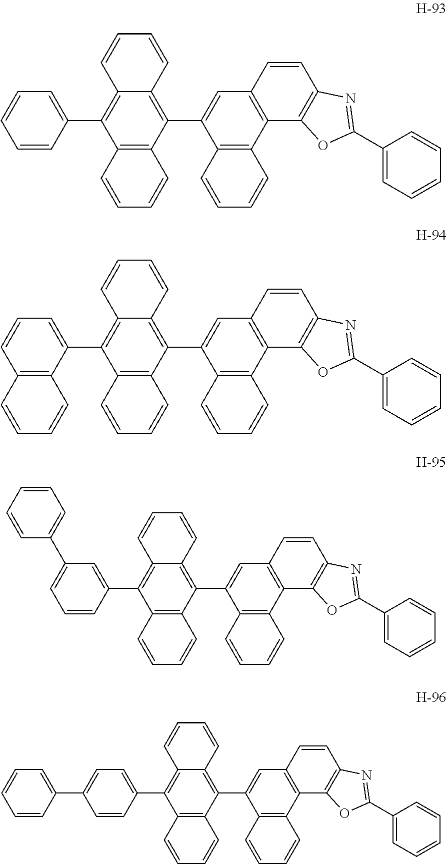 [SMILES: c1ccc(-c2nc3ccc4cc(-c5c6ccccc6c(-c6ccccc6)c6ccccc56)c5ccccc5c4c3o2)cc1 ; c1ccc(-c2nc3ccc4cc(-c5c6ccccc6c(-c6cccc7ccccc67)c6ccccc56)c5ccccc5c4c3o2)cc1 ; c1ccc(-c2cccc(-c3c4ccccc4c(-c4cc5ccc6nc(-c7ccccc7)oc6c5c5ccccc45)c4ccccc34)c2)cc1 ; c1ccc(-c2ccc(-c3c4ccccc4c(-c4cc5ccc6nc(-c7ccccc7)oc6c5c5ccccc45)c4ccccc34)cc2)cc1]

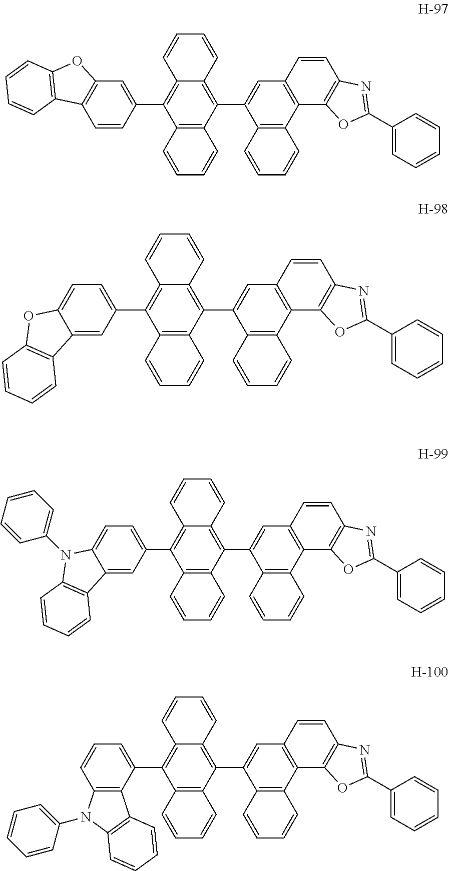 [SMILES: c1ccc(-c2nc3ccc4cc(-c5c6ccccc6c(-c6ccc7c(c6)oc6ccccc67)c6ccccc56)c5ccccc5c4c3o2)cc1 ; c1ccc(-c2nc3ccc4cc(-c5c6ccccc6c(-c6ccc7oc8ccccc8c7c6)c6ccccc56)c5ccccc5c4c3o2)cc1 ; c1ccc(-c2nc3ccc4cc(-c5c6ccccc6c(-c6ccc7c(c6)c6ccccc6n7-c6ccccc6)c6ccccc56)c5ccccc5c4c3o2)cc1 ; c1ccc(-c2nc3ccc4cc(-c5c6ccccc6c(-c6cccc7c6c6ccccc6n7-c6ccccc6)c6ccccc56)c5ccccc5c4c3o2)cc1]

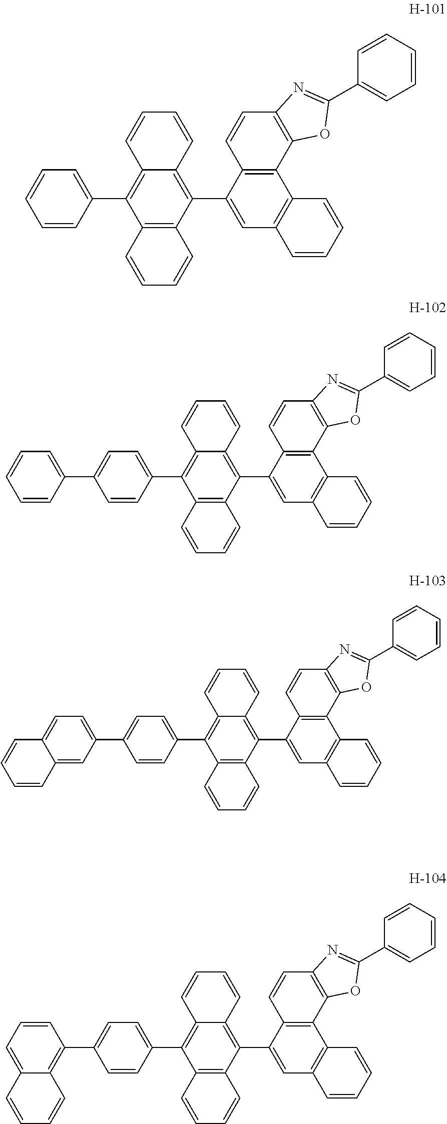 [SMILES: c1ccc(-c2nc3ccc4c(-c5c6ccccc6c(-c6ccccc6)c6ccccc56)cc5ccccc5c4c3o2)cc1 ; c1ccc(-c2ccc(-c3c4ccccc4c(-c4cc5ccccc5c5c4ccc4nc(-c6ccccc6)oc45)c4ccccc34)cc2)cc1 ; c1ccc(-c2nc3ccc4c(-c5c6ccccc6c(-c6ccc(-c7ccc8ccccc8c7)cc6)c6ccccc56)cc5ccccc5c4c3o2)cc1 ; c1ccc(-c2nc3ccc4c(-c5c6ccccc6c(-c6ccc(-c7cccc8ccccc78)cc6)c6ccccc56)cc5ccccc5c4c3o2)cc1]

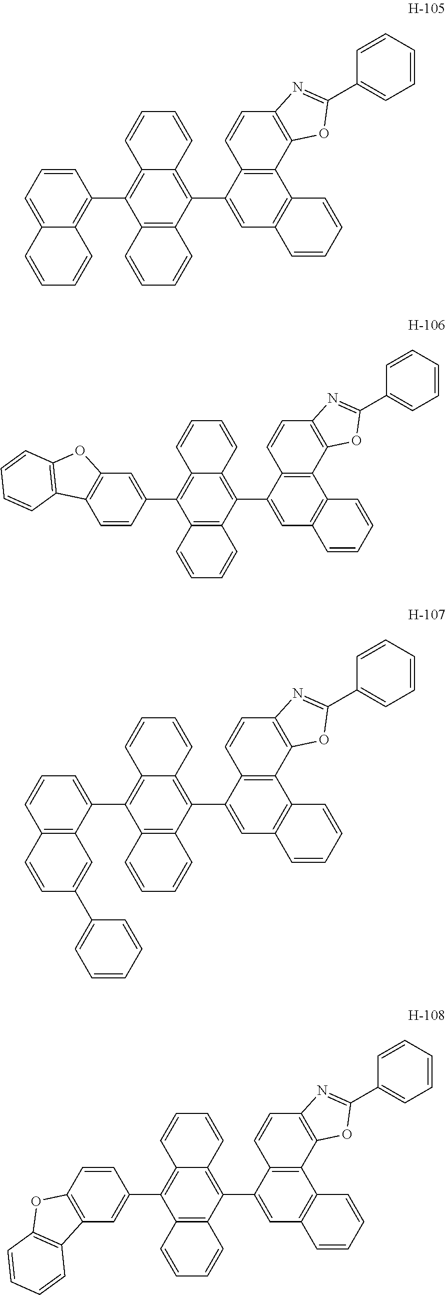 [SMILES: c1ccc(-c2nc3ccc4c(-c5c6ccccc6c(-c6cccc7ccccc67)c6ccccc56)cc5ccccc5c4c3o2)cc1 ; c1ccc(-c2nc3ccc4c(-c5c6ccccc6c(-c6ccc7c(c6)oc6ccccc67)c6ccccc56)cc5ccccc5c4c3o2)cc1 ; c1ccc(-c2ccc3cccc(-c4c5ccccc5c(-c5cc6ccccc6c6c5ccc5nc(-c7ccccc7)oc56)c5ccccc45)c3c2)cc1 ; c1ccc(-c2nc3ccc4c(-c5c6ccccc6c(-c6ccc7oc8ccccc8c7c6)c6ccccc56)cc5ccccc5c4c3o2)cc1]

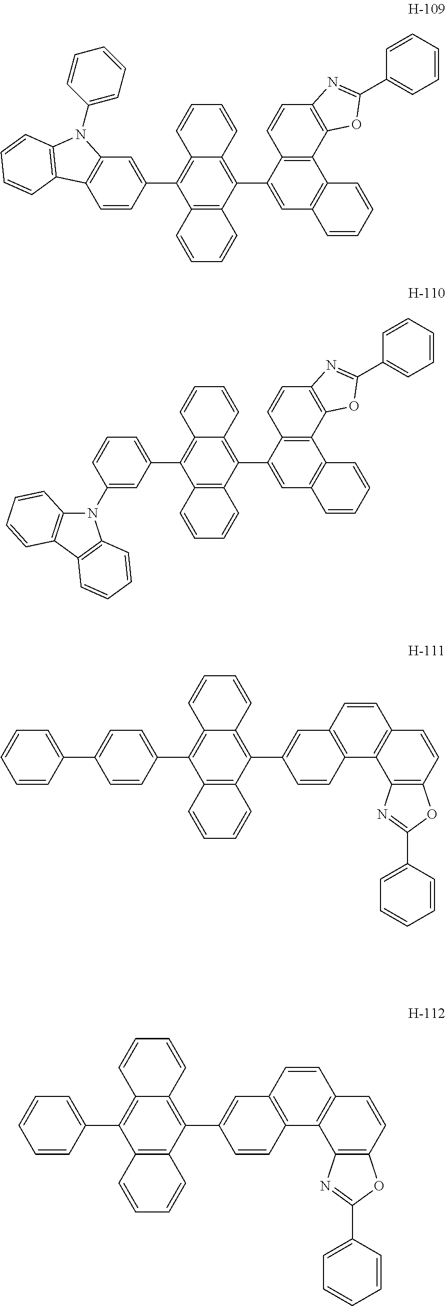 [SMILES: c1ccc(-c2nc3ccc4c(-c5c6ccccc6c(-c6ccc7c8ccccc8n(-c8ccccc8)c7c6)c6ccccc56)cc5ccccc5c4c3o2)cc1 ; c1ccc(-c2nc3ccc4c(-c5c6ccccc6c(-c6cccc(-n7c8ccccc8c8ccccc87)c6)c6ccccc56)cc5ccccc5c4c3o2)cc1 ; c1ccc(-c2ccc(-c3c4ccccc4c(-c4ccc5c(ccc6ccc7oc(-c8ccccc8)nc7c65)c4)c4ccccc34)cc2)cc1 ; c1ccc(-c2nc3c(ccc4ccc5cc(-c6c7ccccc7c(-c7ccccc7)c7ccccc67)ccc5c43)o2)cc1]

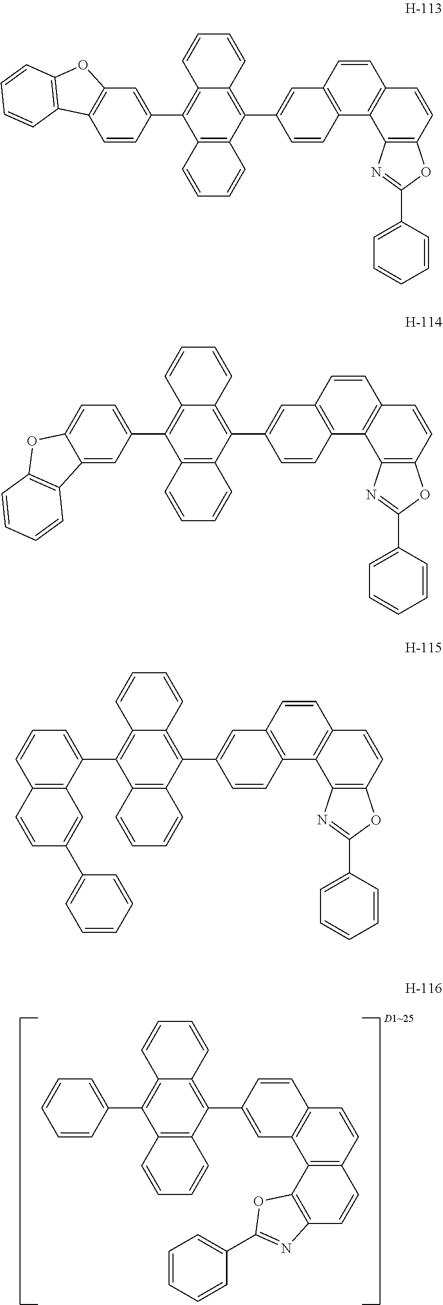

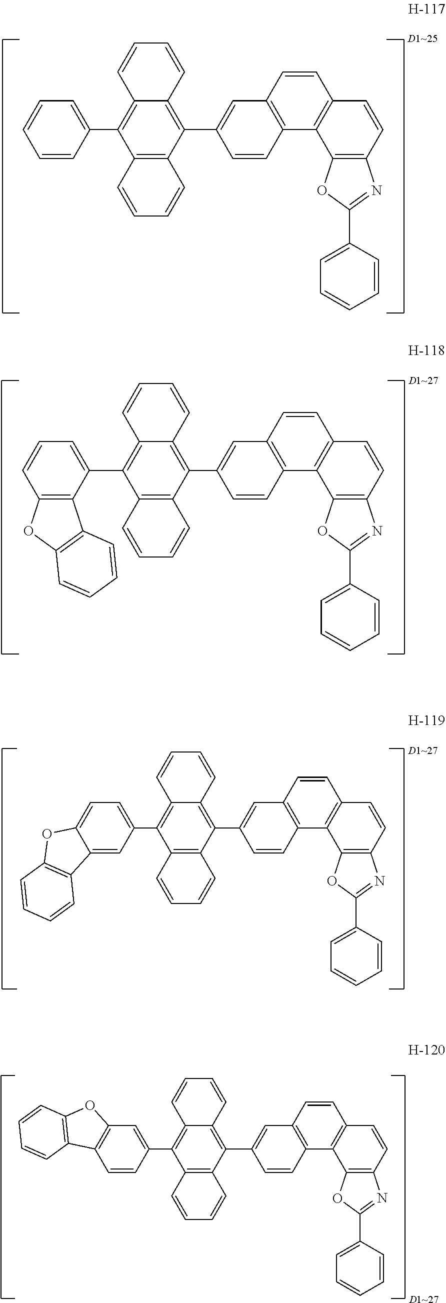

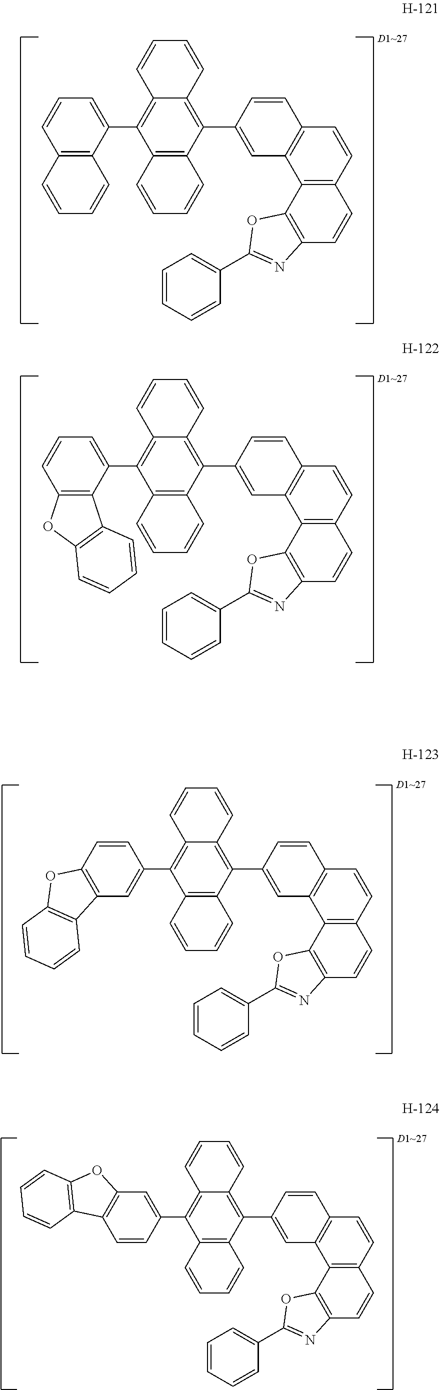

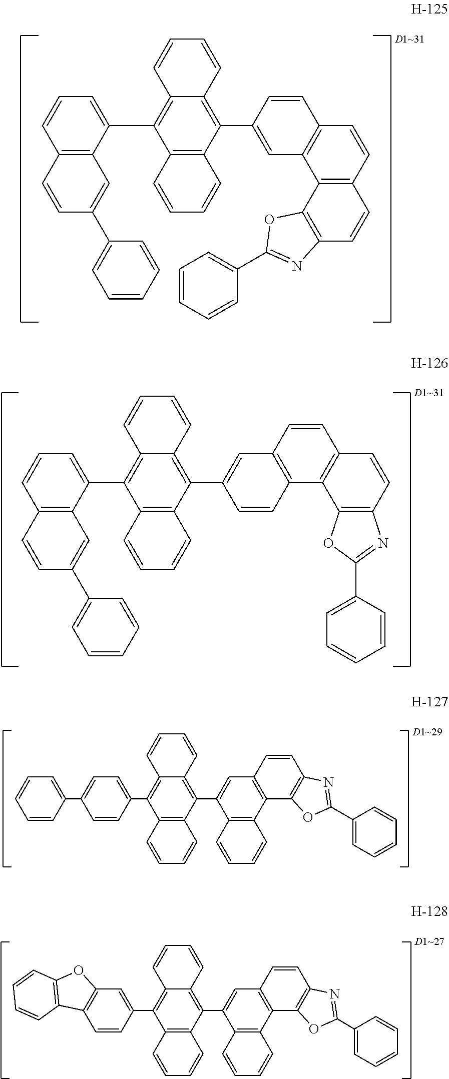

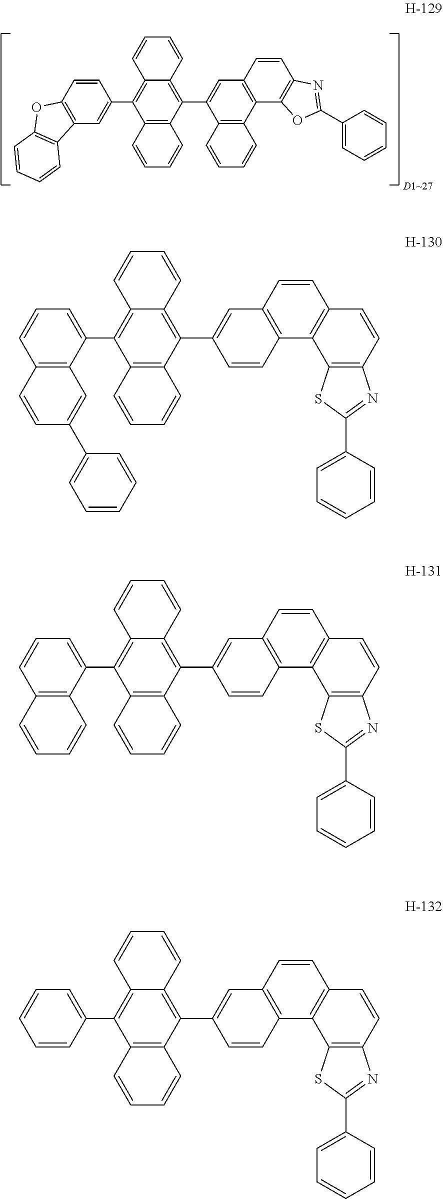

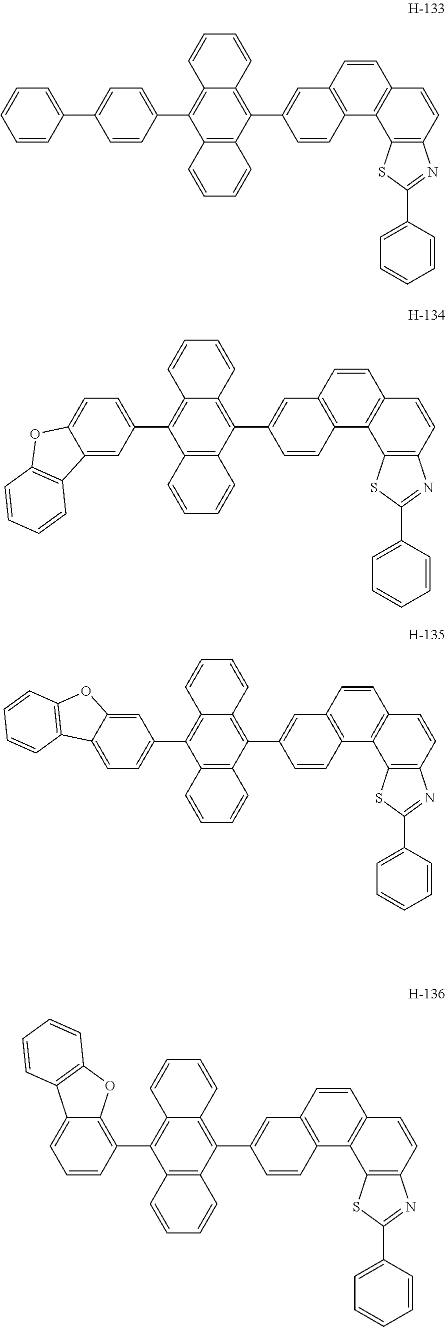 [SMILES: c1ccc(-c2ccc(-c3c4ccccc4c(-c4ccc5c(ccc6ccc7nc(-c8ccccc8)sc7c65)c4)c4ccccc34)cc2)cc1 ; c1ccc(-c2nc3ccc4ccc5cc(-c6c7ccccc7c(-c7ccc8oc9ccccc9c8c7)c7ccccc67)ccc5c4c3s2)cc1 ; c1ccc(-c2nc3ccc4ccc5cc(-c6c7ccccc7c(-c7ccc8c(c7)oc7ccccc78)c7ccccc67)ccc5c4c3s2)cc1 ; c1ccc(-c2nc3ccc4ccc5cc(-c6c7ccccc7c(-c7cccc8c7oc7ccccc78)c7ccccc67)ccc5c4c3s2)cc1]

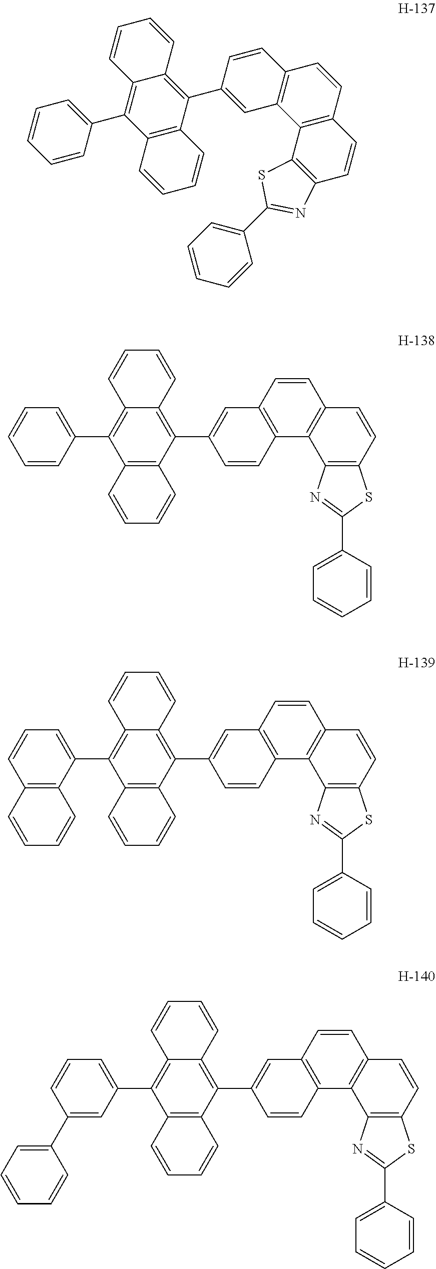 [SMILES: c1ccc(-c2nc3ccc4ccc5ccc(-c6c7ccccc7c(-c7ccccc7)c7ccccc67)cc5c4c3s2)cc1 ; c1ccc(-c2nc3c(ccc4ccc5cc(-c6c7ccccc7c(-c7ccccc7)c7ccccc67)ccc5c43)s2)cc1 ; c1ccc(-c2nc3c(ccc4ccc5cc(-c6c7ccccc7c(-c7cccc8ccccc78)c7ccccc67)ccc5c43)s2)cc1 ; c1ccc(-c2cccc(-c3c4ccccc4c(-c4ccc5c(ccc6ccc7sc(-c8ccccc8)nc7c65)c4)c4ccccc34)c2)cc1]

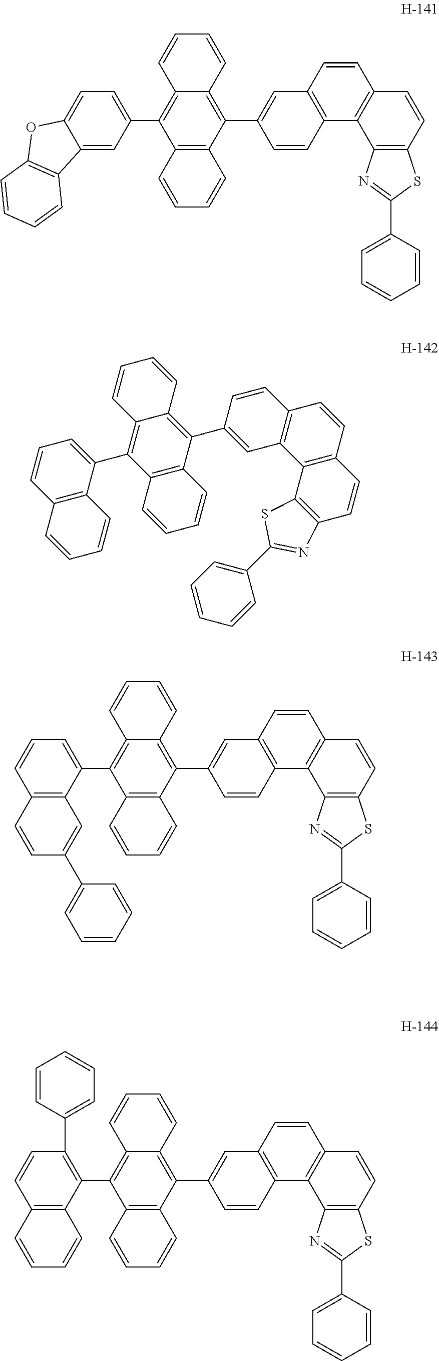 [SMILES: c1ccc(-c2nc3c(ccc4ccc5cc(-c6c7ccccc7c(-c7ccc8oc9ccccc9c8c7)c7ccccc67)ccc5c43)s2)cc1 ; c1ccc(-c2nc3ccc4ccc5ccc(-c6c7ccccc7c(-c7cccc8ccccc78)c7ccccc67)cc5c4c3s2)cc1 ; c1ccc(-c2ccc3cccc(-c4c5ccccc5c(-c5ccc6c(ccc7ccc8sc(-c9ccccc9)nc8c76)c5)c5ccccc45)c3c2)cc1 ; c1ccc(-c2nc3c(ccc4ccc5cc(-c6c7ccccc7c(-c7c(-c8ccccc8)ccc8ccccc78)c7ccccc67)ccc5c43)s2)cc1]

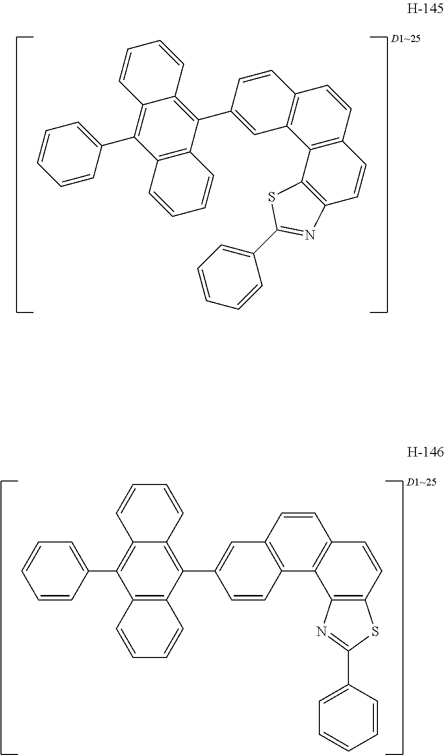

In the compounds above, Dn indicates n hydrogens having been replaced by deuterium. For example, D1˜25 indicates 1 to 25 hydrogens having been replaced by deuterium.

The organic electroluminescent compound according to the present disclosure may be prepared by a synthetic method known to one skilled in the art. For example, the organic electroluminescent compound according to the present disclosure can be prepared by referring to the following reaction schemes 1 and 2, but is not limited thereto.

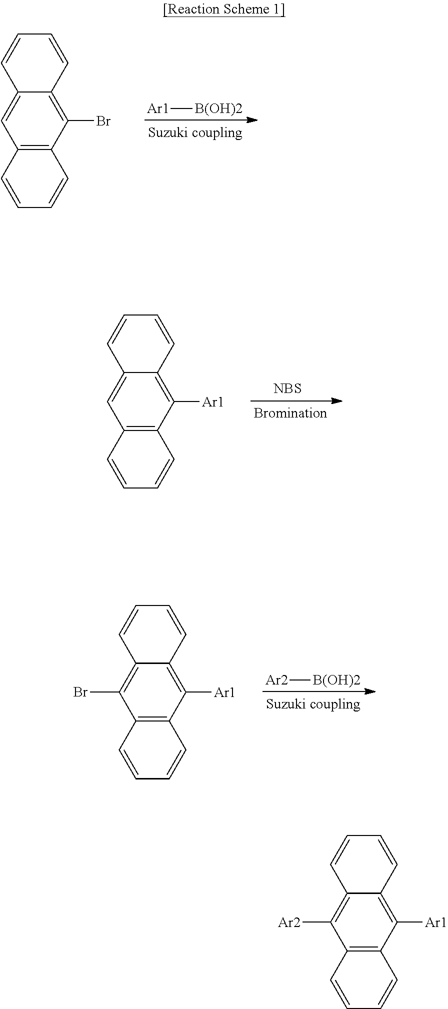

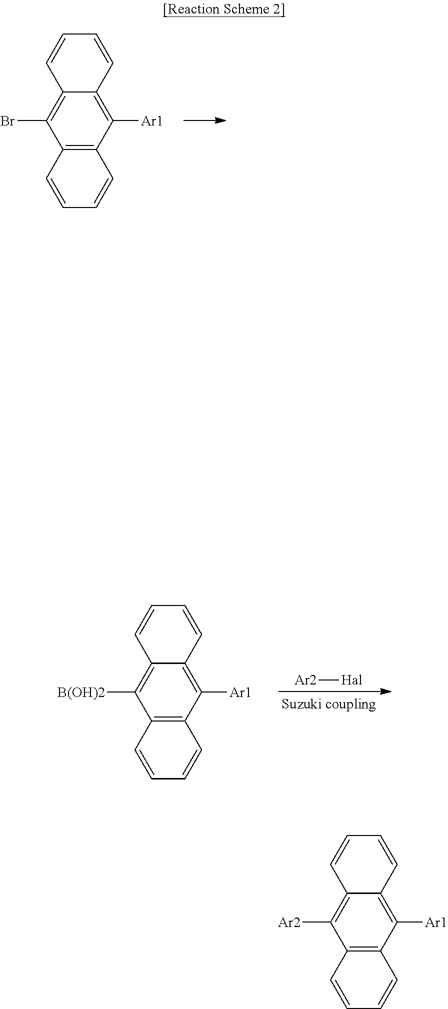

In reaction schemes 1 and 2, any one of Ar₁ and Ar₂ represents a phenanthro-oxazole derivative, and the other has the same definition as Ar above.

Although illustrative synthesis examples of the compound represented by formula 1 were described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, and a Phosphine-mediated reductive cyclization reaction, and the above reactions proceed even when substituents, which are defined in formula 1 above but are not specified in the specific synthesis examples, are bonded.

In addition, the non-deuterated analogues of the compound represented by formula 1 can be prepared by known coupling and substitution reactions. Also, it may be prepared in a similar manner by using deuterated precursor materials, or more generally may be prepared by treating the non-deuterated compound with a deuterated solvent or D6-benzene in the presence of an H/D exchange catalyst such as a Lewis acid, e.g., aluminum trichloride or ethyl aluminum chloride, a trifluoromethanesulfonic acid, or a trifluoromethanesulfonic acid-D.

The dopant that may be used in combination with the compound of the present disclosure may be at least one phosphorescent or fluorescent dopant, preferably at least one phosphorescent dopant. The phosphorescent dopant is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The compound represented by formula 1 of the present disclosure may be comprised in at least one layer consistituting an organic electroluminescent device, and for example, at least one layer selected from a hole injection layer, a hole transport layer, a hole auxiliary layer, a light-emitting auxiliary layer, a light-emitting layer, an electron transport layer, an electron buffer layer, an electron injection layer, an interlayer, a hole blocking layer, and an electron blocking layer. Each of the layers may be additionally composed of several layers. The compound represented by formula 1 of the present disclosure is not limited thereto, but may be included in the light-emitting layer, and may be included in the light-emitting layer as a host material.

The organic electroluminescent materials of the present disclosure, for example, at least one of a hole injection material, a hole transport material, a hole auxiliary material, a light-emitting auxiliary material, an electron blocking material, a light-emitting material, an electron buffer material, a hole blocking material, an electron transport material, and an electron injection layer, may comprise the compound represented by formula 1. The material may be a light-emitting material. The light-emitting material may consist of only the compound represented by formula 1, and may further comprise conventional materials) included in the organic electroluminescent material. When two or more materials are included in one layer, mixed deposition may be performed to form a layer, or co-deposition may be performed separately to form a layer.

The organic electroluminescent device according to the present disclosure comprises a first electrode, a second electrode, and at least one organic layer between the first and second electrodes. One of the first and second electrodes may be an anode, and the other may be a cathode. The organic layer may comprise at least one light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

The first electrode and the second electrode may each be formed with a transmissive conductive material, a transflective conductive material, or a reflective conductive material. The organic electroluminescent device may be a top emission type, a bottom emission type, or both-sides emission type according to the kinds of the material forming the first electrode and the second electrode. In addition, the hole injection layer may be further doped with a p-dopant, and the electron injection layer may be further doped with an n-dopant.

The organic electroluminescent device of the present disclosure may comprise the compound represented by formula 1, and may further comprise conventional material(s) included in the organic electroluminescent device. The organic electroluminescent device comprising the organic electroluminescent compound represented by formula 1 of the present disclosure may exhibit a low operating voltage property.

In addition, the organic electroluminescent material according to one embodiment of the present disclosure may be used as a light-emitting material for a blue organic electroluminescent device. The organic electroluminescent material according to one embodiment of the present disclosure may also be applied to the organic electroluminescent device comprising QD (quantum dot).

The present disclosure may provide a display system by using the compound represented by formula 1. In addition, it is possible to produce a display system or a lighting system by using the compound of the present disclosure. Specifically, it is possible to produce a display system, e.g., a display system for smartphones, tablets, notebooks, PCs, TVs, or cars, or a lighting system, e.g., an outdoor or indoor lighting system, by using the compound of the present disclosure.

Hereinafter, the preparation method of the compound according to the present disclosure and the properties thereof will be explained in detail. However, the present disclosure is not limited to the following examples.

### EXAMPLE 1

### Preparation of Compound H-43

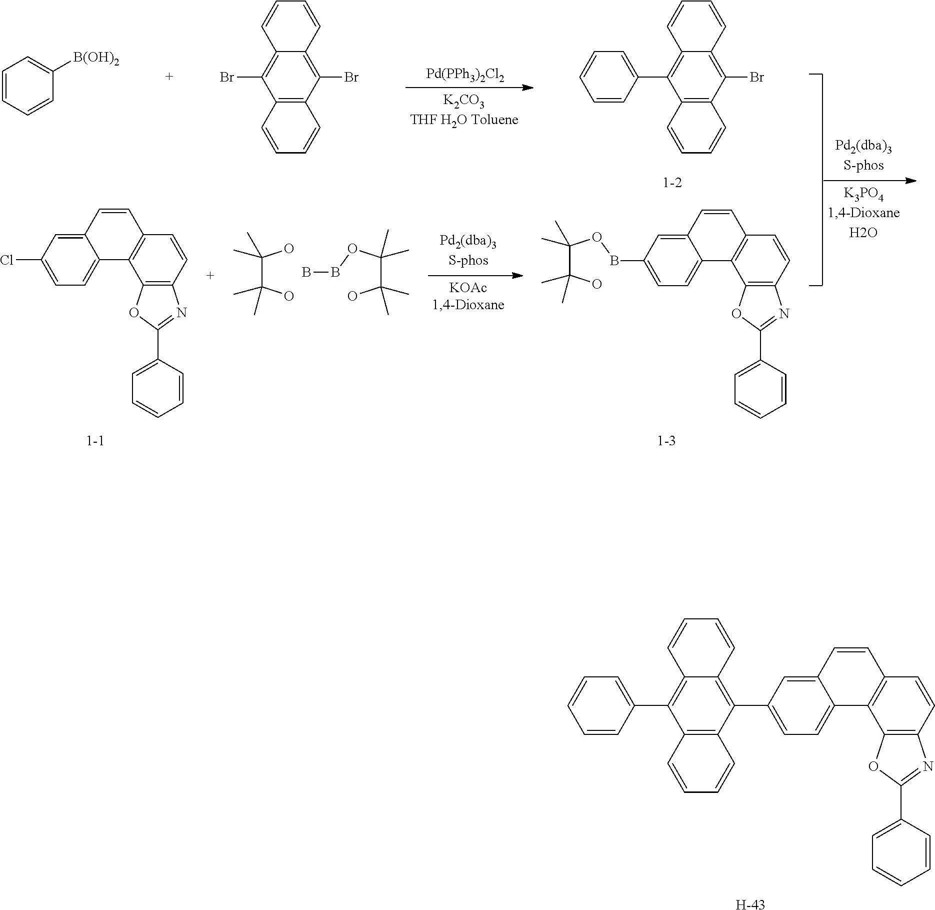

Synthesis of Compound 1 -2

1.21 g of Pd(PPh₃)₂Cl₂ (1.72 mmol), 19.8 g of K₂CO₃ (143.5 mmol), 7 g of phenylboronic acid (57 nmol), and 19.27 g of 9,10-dibromoanthracene (57.41 mmol) were added to 100 mL of tetrahydrofuran, 100 mL of distilled water and 100 mL of toluene, and the mixture was stirred at 70° C. for 12 hours under nitrogen. After completion of the reaction, the water layer was removed, and the organic layer was distilled under reduced pressure. The resulting mixture was separated by column chromatography to obtain 11.68 g of compound 1-2 (yield: 61.5%).

Synthesis of Compound 1-3

1.264 g of Pd₂(dba)₃ (1.38 mmol), 1.133 g of s-phos (2.76 mmol), 10.16 g of KOAc (103.52 mmol), 11.38 g of compound 1-1 (34.51 mmol), and 10.514 g of bis(pinacolato)diborane (41.41 mmol) were added to 250 mL of dioxane, and the mixture was stirred at 100° C. for 12 hours under nitrogen. After completion of the reaction, distilled water was added. The resulting solid was filtered. The obtained solid was separated by column chromatography to obtain 13 g of compound 1-3 (yield: 89.4%).

Synthesis of Compound H-43

1.462 g of Pd₂(dba)₂ (1.60 mmol), 1.31 g of s-phos (3.19 mmol), 16.92 g of K₃PO₄ (79.81 mmol), 10.74 g of compound 1-2 (32.24 mmol), and 13.45 g of compound 1-3 (31.92mmol) were added to 100 mL of 1,4-dioxane, 100 mL of distilled water, and 100 mL of toluene, and the mixture was stirred at 100° C. for 12 hours under nitrogen. After completion of the reaction, the water layer was removed, and the organic layer was distilled under reduced pressure. The obtained solid was separated by column chromatography to obtain 15.7 g of compound H-43 (yield: 89.9%).

| MW | M.P. |
|---|---|
| 547.6 | 332° C. |

### EXAMPLE 2

### Synthesis of Compound H-1

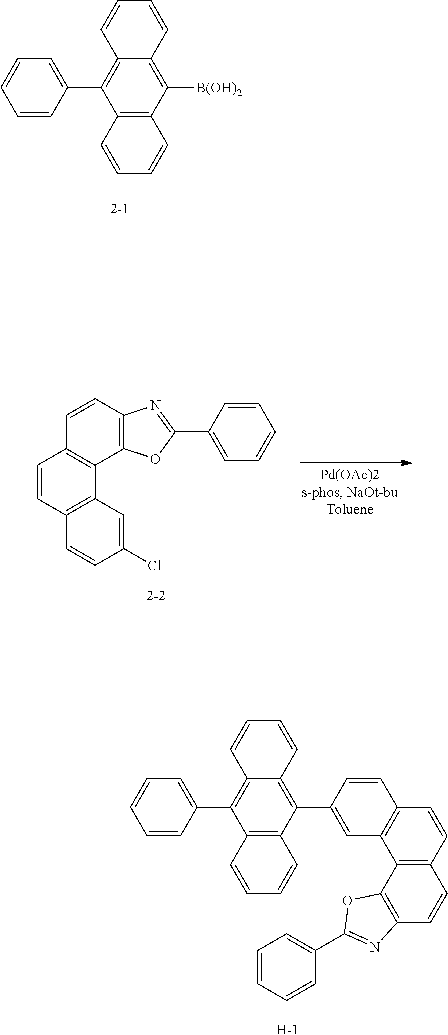

In a reaction container, 7.6 g of compound 2-1 (25.5 mmol), 6 g of compound 2-2 (18.2 mmol), 0.4 g of Pd(OAc)₂ (1.8 mmol), 1.47 g of s-phos (3,6 mmol), and 3.49 g of NaOt-bu (36.4 mmol) were added to 250 mL of toluene, and the mixture was stirred under reflux. After 2 hours, the reaction mixture was cooled to room temperature, and extracted with dichloromethane. The organic layer was washed with distilled water. The obtained organic layer was distilled under reduced pressure, and the residue was separated by column chromatography (chlorobenzene:chloroform=1:0 to 0:1) to obtain 8.7 g of compound H-1 (yield: 87.4%).

| MW | M.P. |
|---|---|
| 547.19 | 320.9° C. |

### EXAMPLE 3

### Synthesis of Compound H-29

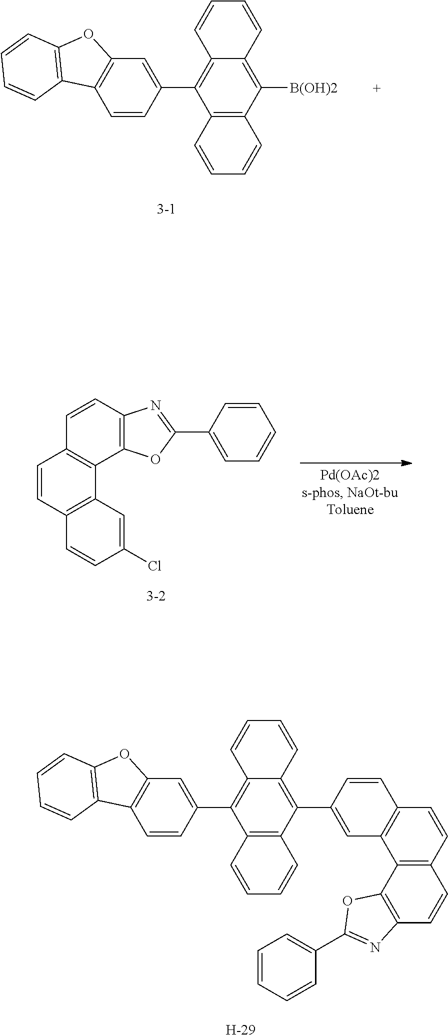

In a reaction container, 5.1 g of compound 3-1 (13.13 mmol), 6 g of compound 3-2 (18.2 mmol), 0.4 g of Pd(OAc)₂ (1.8 mmol), 1.47 g of s-phos (3.6 mmol), and 3.49 g of NaOt-bu (36.4 mmol) were added to 250 mL of toluene, and the mixture was stirred under reflux. After 2 hours, the reaction mixture was cooled to room temperature, and extracted with dichloromethane. The organic layer was washed with distilled water. The obtained organic layer was distilled under reduced pressure, and the residue was separated by column chromatography (chlorobenzene:chloroform=1:0 to 0:1) to obtain 1.1 g of compound H-29 (yield: 13.1%).

| MW | M.P. |
|---|---|
| 637.74 | 358° C. |

Hereinafter, the properties of an OLED comprising the compound according to the present disclosure will be explained. However, the following examples merely illustrate the properties of an OLED according to the present disclosure in detail, but the present disclosure is not limited to the following examples.

### DEVICE EXAMPLE 1

### Producing an OLED Using the Compound According to the Present Disclosure

An OLED was produced using the organic electroluminescent compound according to the present disclosure, as follows: A transparent electrode indium tin oxide (ITO) thin film (10 Ω/sq) on a glass substrate for an OLED (GEOMATEC CO., LTD., Japan) was subjected to an ultrasonic washing with acetone, ethanol and distilled water, sequentially, and then was stored in isopropanol. The ITO substrate was mounted on a substrate holder of a vacuum vapor deposition apparatus. Compound HI-1 was introduced into a cell of the vacuum vapor deposition apparatus, and the pressure in the chamber of the apparatus was then controlled to 10⁻⁶ torr. Thereafter, an electric current was applied to the cell to evaporate the above-introduced material, thereby forming a first hole injection layer having a thickness of 60 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into another cell of the vacuum vapor deposition apparatus and was evaporated by applying an electric current to the cell, thereby forming a first hole transport layer having a thickness of 20 nm on the second hole injection layer. Compound HT-2 was then introduced into another cell of the vacuum vapor deposition apparatus and was evaporated by applying an electric current to the cell, thereby forming a second hole transport layer having a thickness of 5 nm on the first hole transport layer. After forming the hole injection layers and the hole transport layers, a light-emitting layer was formed thereon as follows: Compound H-43 was introduced into one cell of the vacuum vapor depositing apparatus as a host and compound BD was introduced into another cell as a dopant. The two materials were evaporated and the dopant was deposited in a doping amount of 2 wt % based on the total amount of the host and dopant to form a light-emitting layer having a thickness of 20 nm on the second hole transport layer. Next, compound ET-1 and compound EI-1 were evaporated at a rate of 1:1 in two other cells to deposit an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### DEVICE EXAMPLE 2

### Producing an OLED Using the Compound According to the Present Disclosure

An OLED was produced in the same manner as in Device Example 1, except that compound H-1 was used as a host material of the light-emitting layer.

### COMPARATIVE EXAMPLE 1

### Producing an OLED Using a Conventional Compound

An OLED was produced in the same manner as in Device Example 1, except that compound BH-1 was used as a host material of the light-emitting layer.

### COMPARATIVE EXAMPLE 2

### Producing an OLED Using a Conventional Compound

An OLED was produced in the same manner as in Device Example 1, except that compound BH-2 was used as a host material of the light-emitting layer.

The compounds used in the Device Examples and the Comparative Examples are as follows.

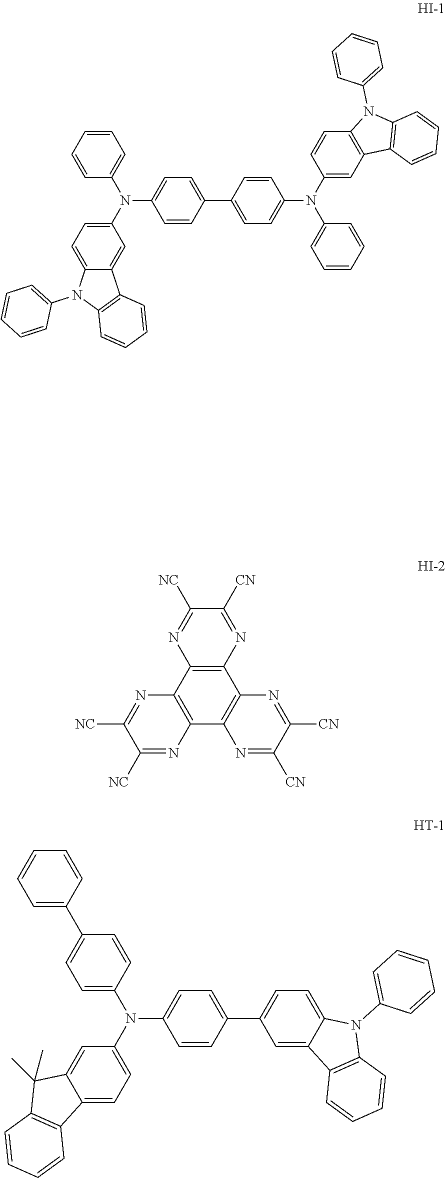 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21]

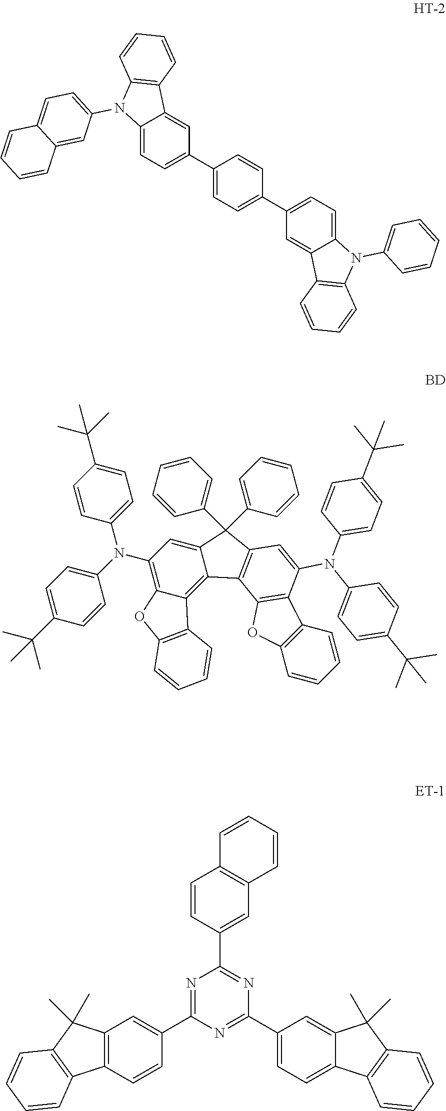 [SMILES: c1ccc(-n2c3ccccc3c3cc(-c4ccc(-c5ccc6c(c5)c5ccccc5n6-c5ccc6ccccc6c5)cc4)ccc32)cc1 ; CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C3(c2ccccc2)c2ccccc2)cc1 ; CC1(C)c2ccccc2-c2ccc(-c3nc(-c4ccc5c(c4)C(C)(C)c4ccccc4-5)nc(-c4ccc5ccccc5c4)n3)cc21]

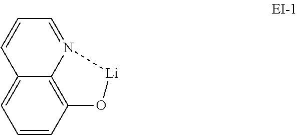

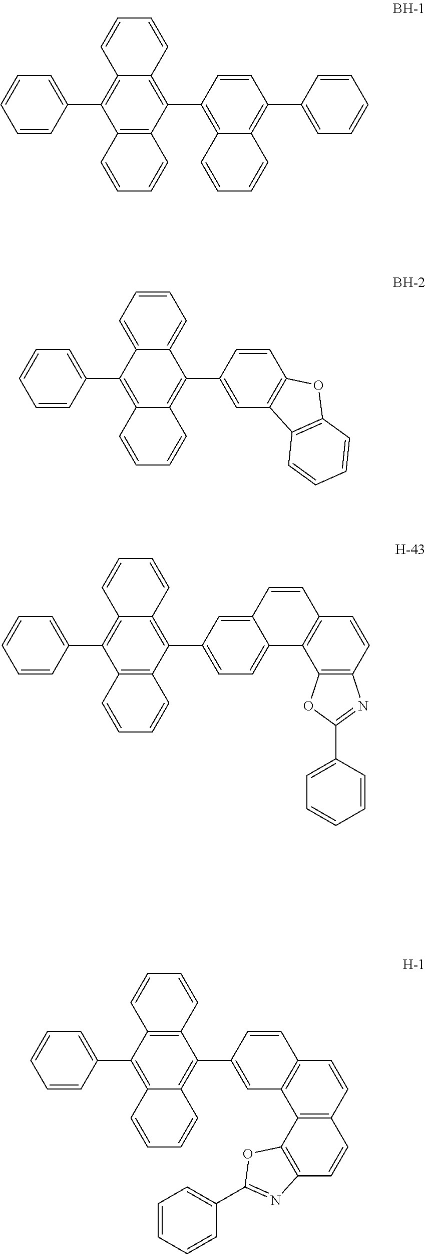

The results of the the operating voltage, luminous efficiency, and CIE color coordinates at a luminance of 1,000 nits of the OLEDs produced in the Device Examples and the Comparative Examples, are shown in the following Table 1.

TABLE 1

| | | | Luminous | | |
|---|---|---|---|---|---|
| | | Operating | Efficiency | CIE | |
| | Host | Voltage [V] | [cd/A] | x | y |
| Device Example 1 | H-43 | 3.4 | 8.5 | 0.138 | 0.111 |
| Device Example 2 | H-1 | 3.4 | 8.4 | 0.138 | 0.117 |
| Comparative | BH-1 | 4.3 | 8.4 | 0.138 | 0.102 |
| Example 1 | | | | | |
| Comparative | BH-2 | 4.0 | 7.8 | 0.137 | 0.106 |
| Example 2 | | | | | |

From the above results, it was confirmed that an organic electroluminescent device comprising a compound having both a phenanthro-oxazole structure and an anthracenyl structure as the host in the light-emitting layer has an operating voltage lower than a conventional organic electroluminescent device. According to the present disclosure, a competitive operating voltage of a blue device, which may be balanced with the operating voltages with red- and green-devices, can be ensured in order to apply it to various applications such as a display.

1. An organic electroluminescent compound represented by the following formula 1: 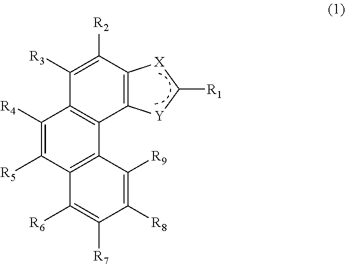
wherein
X represents —N═, —NR—, —O—, or —S—;
Y represents —N═, —NR—, —O—, or —S—; with the proviso that one of X and Y represents —N═, and the other of X and Y represents —NR—, —O—, or —S—;
R represents hydrogen, deuterium, a halogen, a cyano, a substituted or unsubstituted (C1-C30)alkyl, a substituted or unsubstituted (C6-C30)aryl, or a substituted or unsubstituted (5- to 30-membered)heteroaryl; and
R₁ to R₉, each independently, represent hydrogen, deuterium, a halogen, a cyano, a substituted or unsubstituted (C1-C30)alkyl, a substituted or unsubstituted (C6-C30)aryl, or a substituted or unsubstituted (5- to 30-membered)heteroaryl; with the proviso that at least one of R₁ to R9 represents a substituted or unsubstituted anthracenyl.

2. The organic electroluminescent compound according to claim 1, wherein formula 1 is represented by any one of the following formulas 1-1 to 1-9: 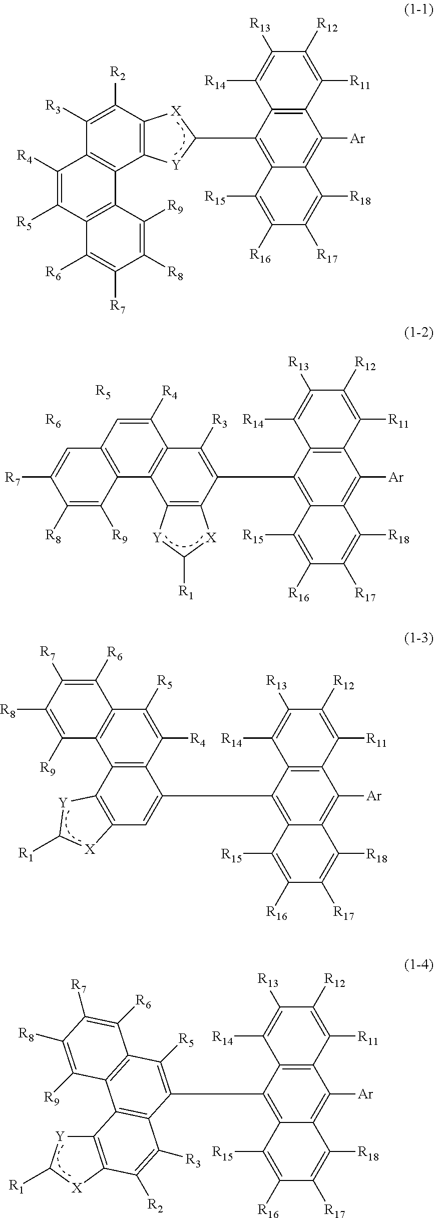 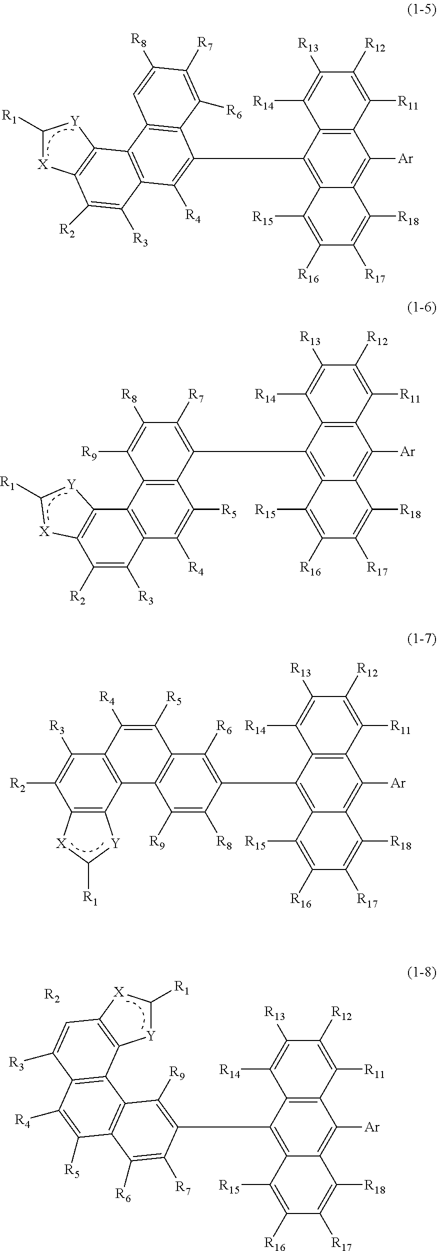 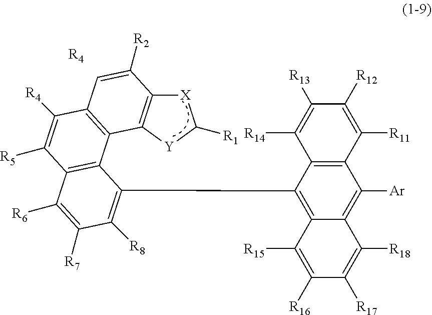
wherein
R₁₁ to R₁₈, each independently, represent hydrogen, deuterium, a halogen, a cyano, a substituted or unsubstituted (C1-C30)alkyl, a substituted or unsubstituted (C6-C30)aryl, or a substituted or unsubstituted (5- to 30-membered)heteroaryl;
Ar represents a substituted or unsubstituted (C6-C30)aryl, or a substituted or unsubstituted (5- to 30-membered)heteroaryl; and
R₁ to R₉, X, and Y are as defined in claim 1.

3. The organic electroluminescent compound according to claim 1, wherein the substituents of the substituted alkyl, the substituted aryl, the substituted heteroaryl, and the substituted anthracenyl, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (5- to 30-membered)heteroaryl unsubstituted or substituted with a (C6-C30)aryl; a (C6-C30)aryl unsubstituted or substituted with a (5- to 30-membered)heteroaryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl; a (C1-C30)alkyl(C6-C30)arylamino; a (C1-C30)alkylcarbonyl; a (C1-C30)alkoxycarbonyl; a (C6-C30)arylcarbonyl: a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

4. The organic electroluminescent compound according to claim 1, wherein the compound represented by the formula 1 is any one selected from the group consisting of the following compounds:

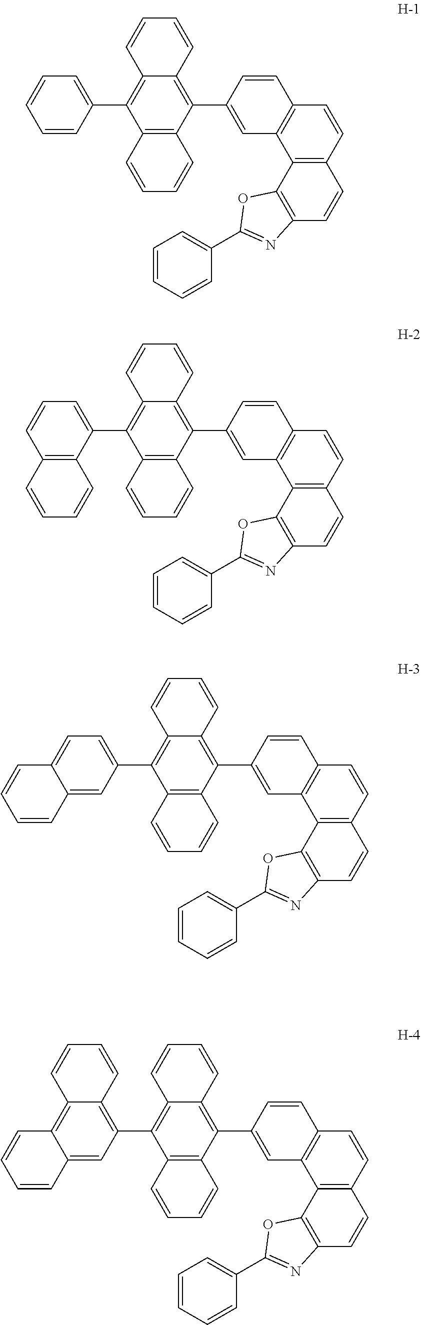

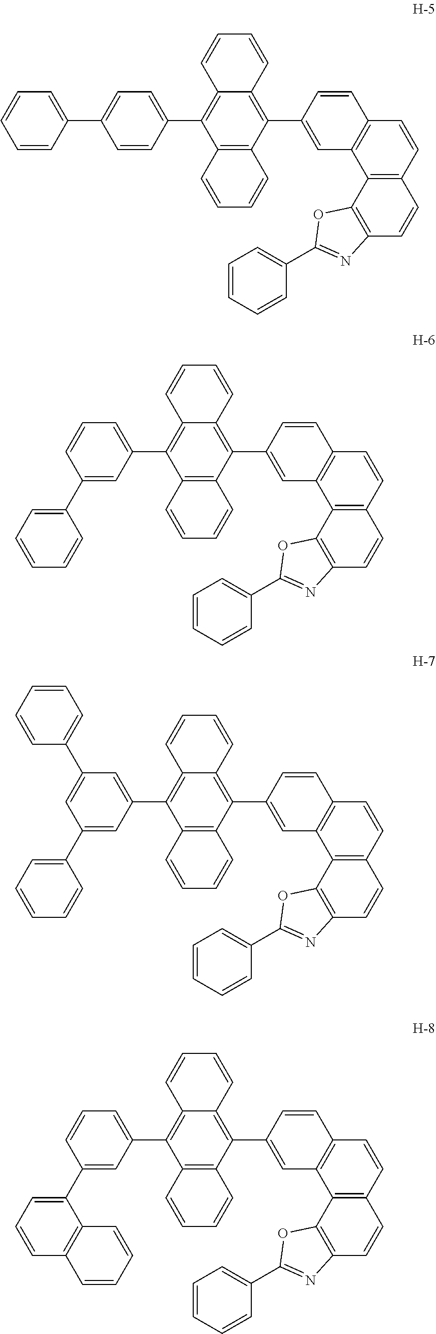 [SMILES: c1ccc(-c2ccc(-c3c4ccccc4c(-c4ccc5ccc6ccc7nc(-c8ccccc8)oc7c6c5c4)c4ccccc34)cc2)cc1 ; c1ccc(-c2cccc(-c3c4ccccc4c(-c4ccc5ccc6ccc7nc(-c8ccccc8)oc7c6c5c4)c4ccccc34)c2)cc1 ; c1ccc(-c2cc(-c3ccccc3)cc(-c3c4ccccc4c(-c4ccc5ccc6ccc7nc(-c8ccccc8)oc7c6c5c4)c4ccccc34)c2)cc1 ; c1ccc(-c2nc3ccc4ccc5ccc(-c6c7ccccc7c(-c7cccc(-c8cccc9ccccc89)c7)c7ccccc67)cc5c4c3o2)cc1]

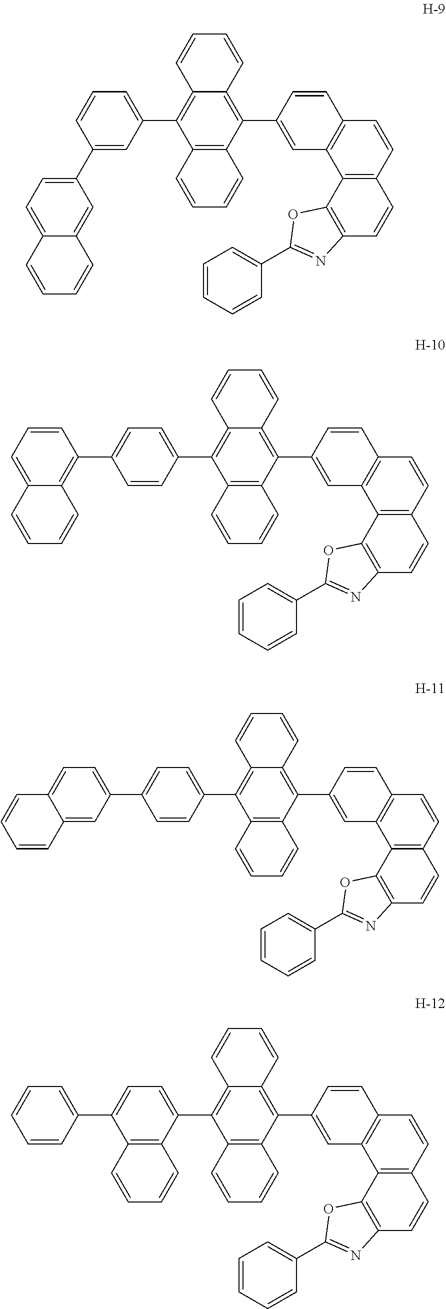 [SMILES: c1ccc(-c2nc3ccc4ccc5ccc(-c6c7ccccc7c(-c7cccc(-c8ccc9ccccc9c8)c7)c7ccccc67)cc5c4c3o2)cc1 ; c1ccc(-c2nc3ccc4ccc5ccc(-c6c7ccccc7c(-c7ccc(-c8cccc9ccccc89)cc7)c7ccccc67)cc5c4c3o2)cc1 ; c1ccc(-c2nc3ccc4ccc5ccc(-c6c7ccccc7c(-c7ccc(-c8ccc9ccccc9c8)cc7)c7ccccc67)cc5c4c3o2)cc1 ; c1ccc(-c2nc3ccc4ccc5ccc(-c6c7ccccc7c(-c7ccc(-c8ccccc8)c8ccccc78)c7ccccc67)cc5c4c3o2)cc1]

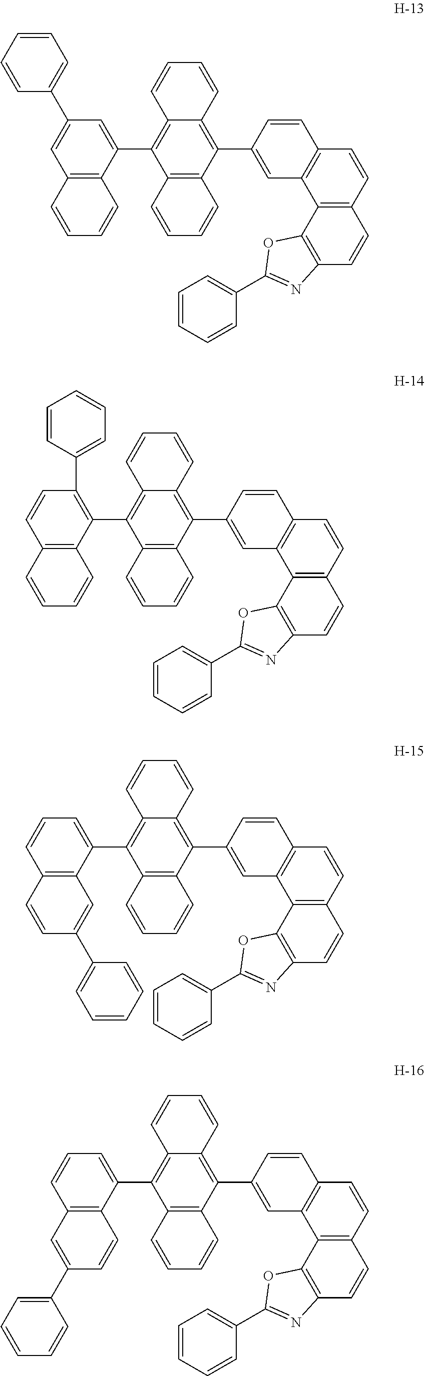 [SMILES: c1ccc(-c2cc(-c3c4ccccc4c(-c4ccc5ccc6ccc7nc(-c8ccccc8)oc7c6c5c4)c4ccccc34)c3ccccc3c2)cc1 ; c1ccc(-c2nc3ccc4ccc5ccc(-c6c7ccccc7c(-c7c(-c8ccccc8)ccc8ccccc78)c7ccccc67)cc5c4c3o2)cc1 ; c1ccc(-c2ccc3cccc(-c4c5ccccc5c(-c5ccc6ccc7ccc8nc(-c9ccccc9)oc8c7c6c5)c5ccccc45)c3c2)cc1 ; c1ccc(-c2ccc3c(-c4c5ccccc5c(-c5ccc6ccc7ccc8nc(-c9ccccc9)oc8c7c6c5)c5ccccc45)cccc3c2)cc1]

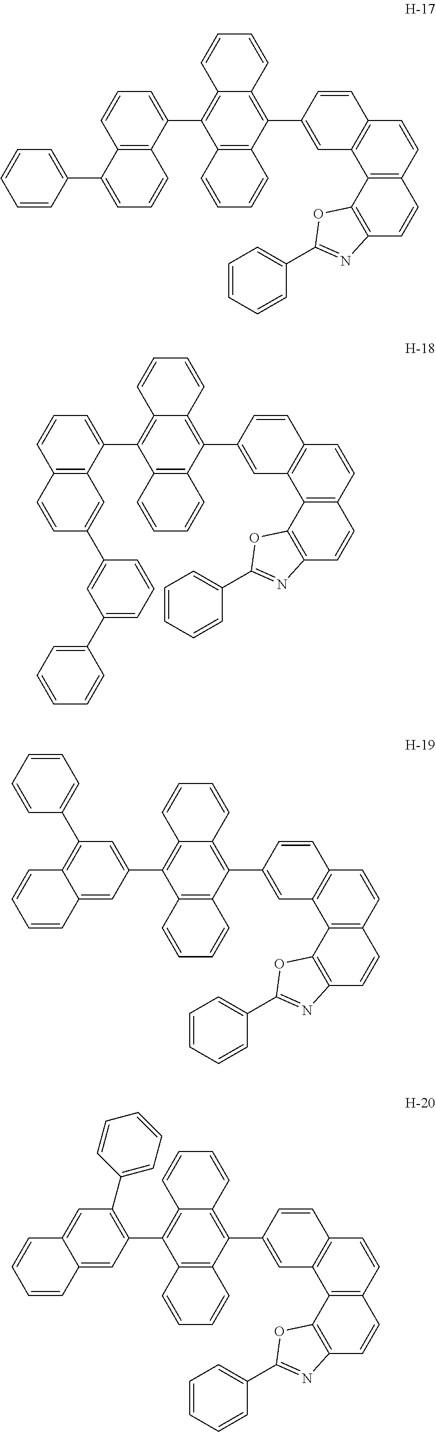 [SMILES: c1ccc(-c2nc3ccc4ccc5ccc(-c6c7ccccc7c(-c7cccc8c(-c9ccccc9)cccc78)c7ccccc67)cc5c4c3o2)cc1 ; c1ccc(-c2cccc(-c3ccc4cccc(-c5c6ccccc6c(-c6ccc7ccc8ccc9nc(-c%10ccccc%10)oc9c8c7c6)c6ccccc56)c4c3)c2)cc1 ; c1ccc(-c2nc3ccc4ccc5ccc(-c6c7ccccc7c(-c7cc(-c8ccccc8)c8ccccc8c7)c7ccccc67)cc5c4c3o2)cc1 ; c1ccc(-c2nc3ccc4ccc5ccc(-c6c7ccccc7c(-c7cc8ccccc8cc7-c7ccccc7)c7ccccc67)cc5c4c3o2)cc1]

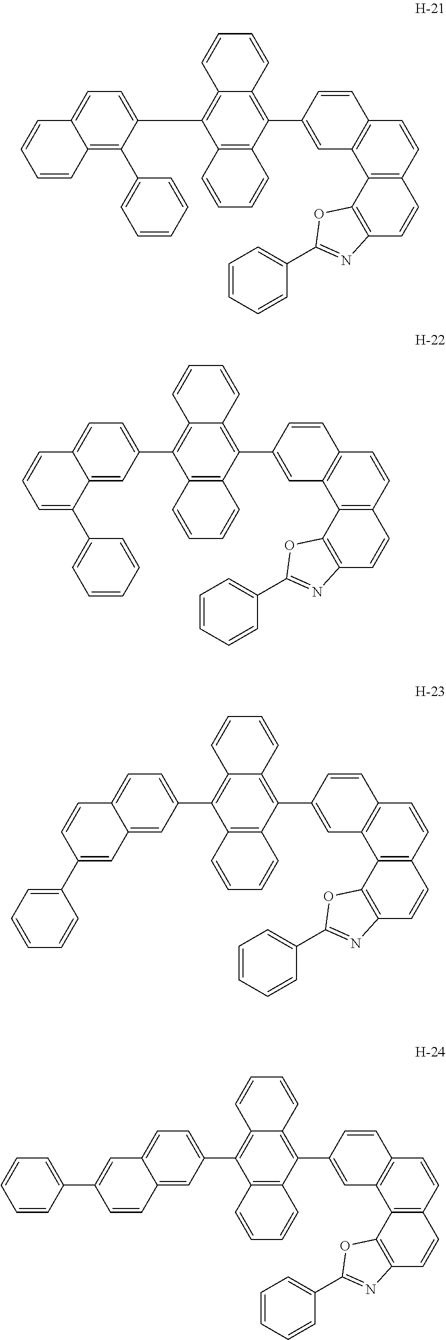 [SMILES: c1ccc(-c2nc3ccc4ccc5ccc(-c6c7ccccc7c(-c7ccc8ccccc8c7-c7ccccc7)c7ccccc67)cc5c4c3o2)cc1 ; c1ccc(-c2nc3ccc4ccc5ccc(-c6c7ccccc7c(-c7ccc8cccc(-c9ccccc9)c8c7)c7ccccc67)cc5c4c3o2)cc1 ; c1ccc(-c2ccc3ccc(-c4c5ccccc5c(-c5ccc6ccc7ccc8nc(-c9ccccc9)oc8c7c6c5)c5ccccc45)cc3c2)cc1 ; c1ccc(-c2ccc3cc(-c4c5ccccc5c(-c5ccc6ccc7ccc8nc(-c9ccccc9)oc8c7c6c5)c5ccccc45)ccc3c2)cc1]

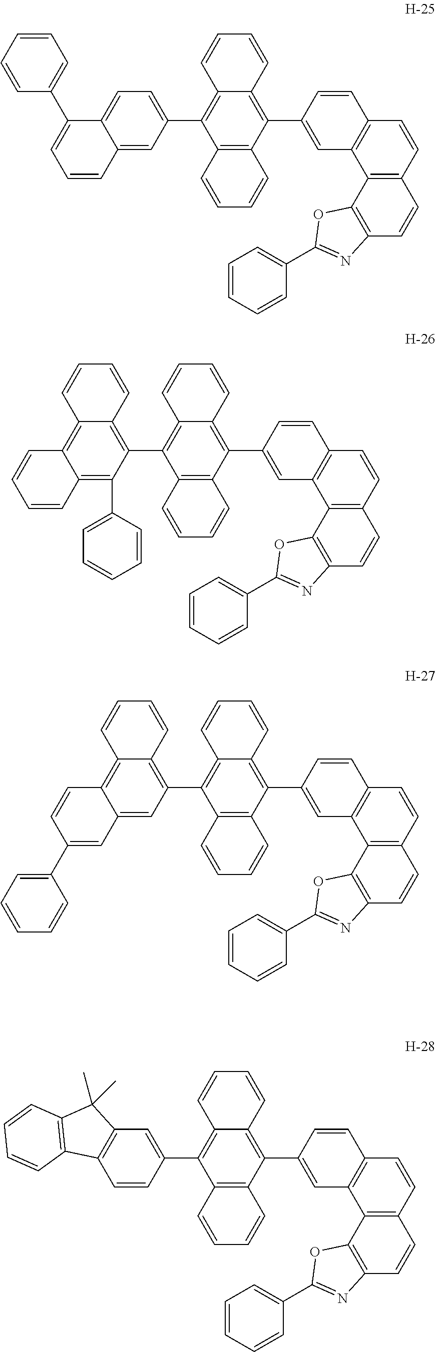 [SMILES: c1ccc(-c2nc3ccc4ccc5ccc(-c6c7ccccc7c(-c7ccc8c(-c9ccccc9)cccc8c7)c7ccccc67)cc5c4c3o2)cc1 ; c1ccc(-c2nc3ccc4ccc5ccc(-c6c7ccccc7c(-c7c(-c8ccccc8)c8ccccc8c8ccccc78)c7ccccc67)cc5c4c3o2)cc1 ; c1ccc(-c2ccc3c(c2)cc(-c2c4ccccc4c(-c4ccc5ccc6ccc7nc(-c8ccccc8)oc7c6c5c4)c4ccccc24)c2ccccc23)cc1 ; CC1(C)c2ccccc2-c2ccc(-c3c4ccccc4c(-c4ccc5ccc6ccc7nc(-c8ccccc8)oc7c6c5c4)c4ccccc34)cc21]

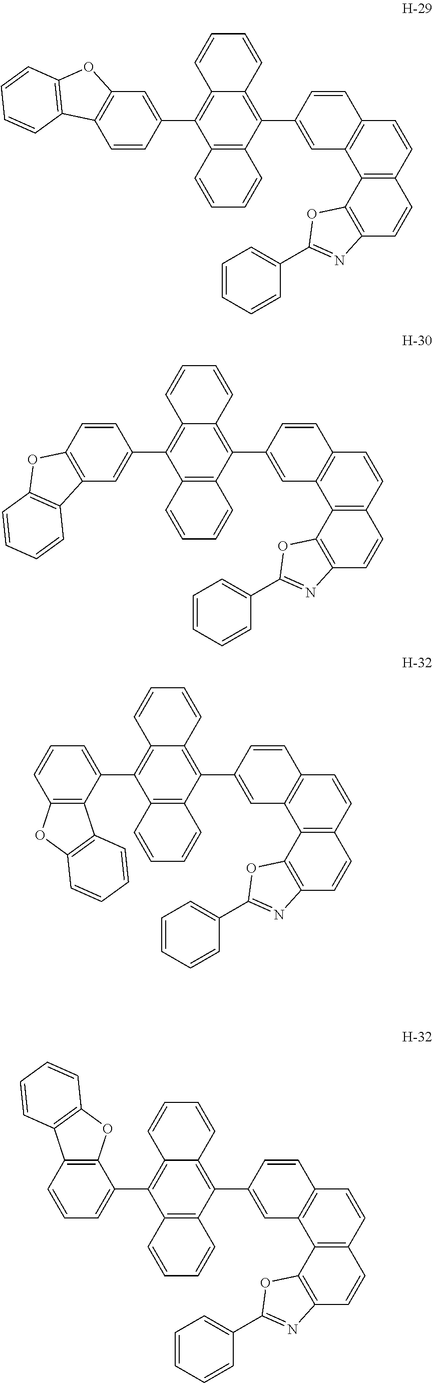 [SMILES: c1ccc(-c2nc3ccc4ccc5ccc(-c6c7ccccc7c(-c7ccc8c(c7)oc7ccccc78)c7ccccc67)cc5c4c3o2)cc1 ; c1ccc(-c2nc3ccc4ccc5ccc(-c6c7ccccc7c(-c7ccc8oc9ccccc9c8c7)c7ccccc67)cc5c4c3o2)cc1 ; c1ccc(-c2nc3ccc4ccc5ccc(-c6c7ccccc7c(-c7cccc8oc9ccccc9c78)c7ccccc67)cc5c4c3o2)cc1 ; c1ccc(-c2nc3ccc4ccc5ccc(-c6c7ccccc7c(-c7cccc8c7oc7ccccc78)c7ccccc67)cc5c4c3o2)cc1]

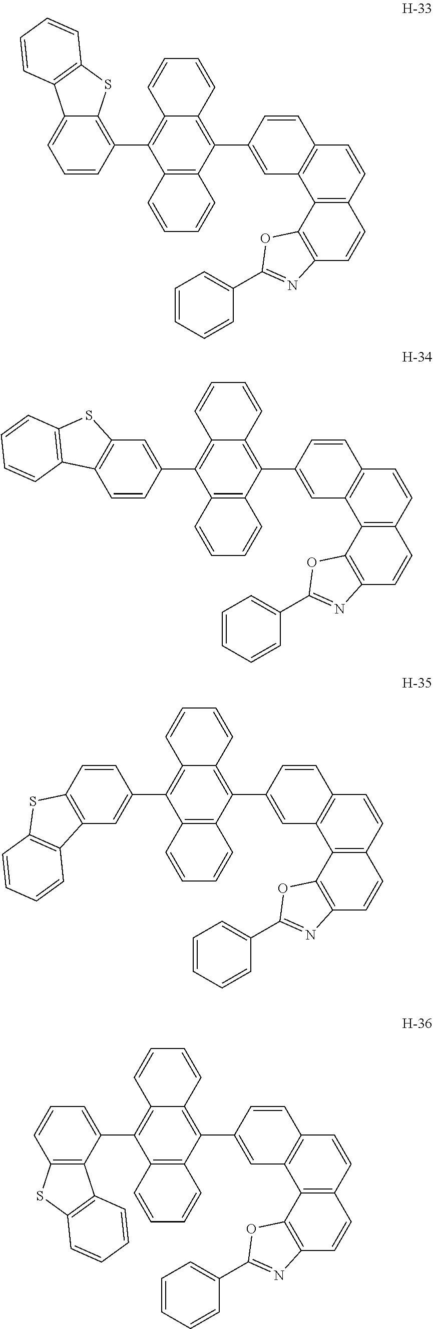

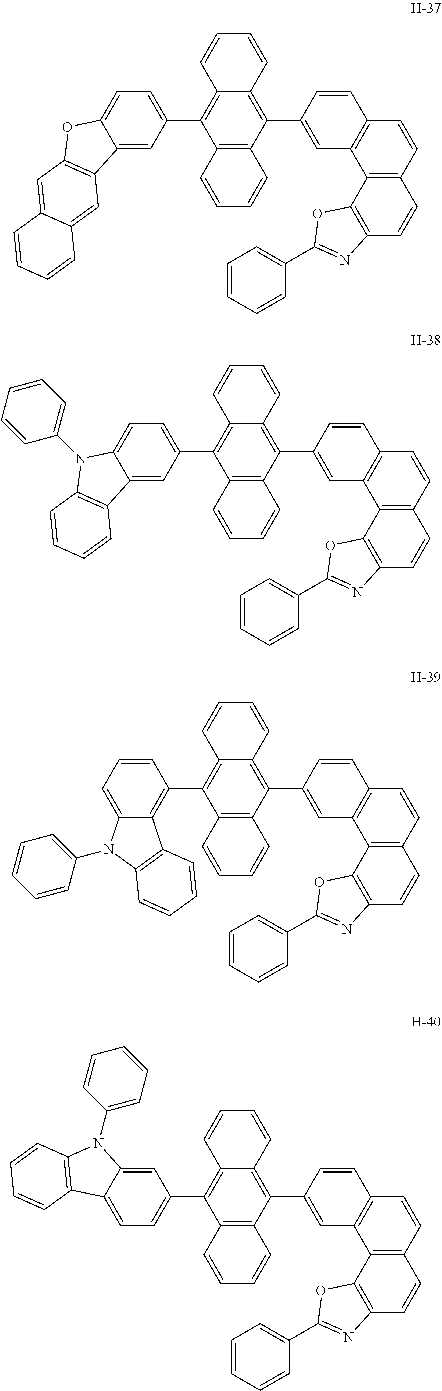 [SMILES: c1ccc(-c2nc3ccc4ccc5ccc(-c6c7ccccc7c(-c7ccc8oc9cc%10ccccc%10cc9c8c7)c7ccccc67)cc5c4c3o2)cc1 ; c1ccc(-c2nc3ccc4ccc5ccc(-c6c7ccccc7c(-c7ccc8c(c7)c7ccccc7n8-c7ccccc7)c7ccccc67)cc5c4c3o2)cc1 ; c1ccc(-c2nc3ccc4ccc5ccc(-c6c7ccccc7c(-c7cccc8c7c7ccccc7n8-c7ccccc7)c7ccccc67)cc5c4c3o2)cc1 ; c1ccc(-c2nc3ccc4ccc5ccc(-c6c7ccccc7c(-c7ccc8c9ccccc9n(-c9ccccc9)c8c7)c7ccccc67)cc5c4c3o2)cc1]

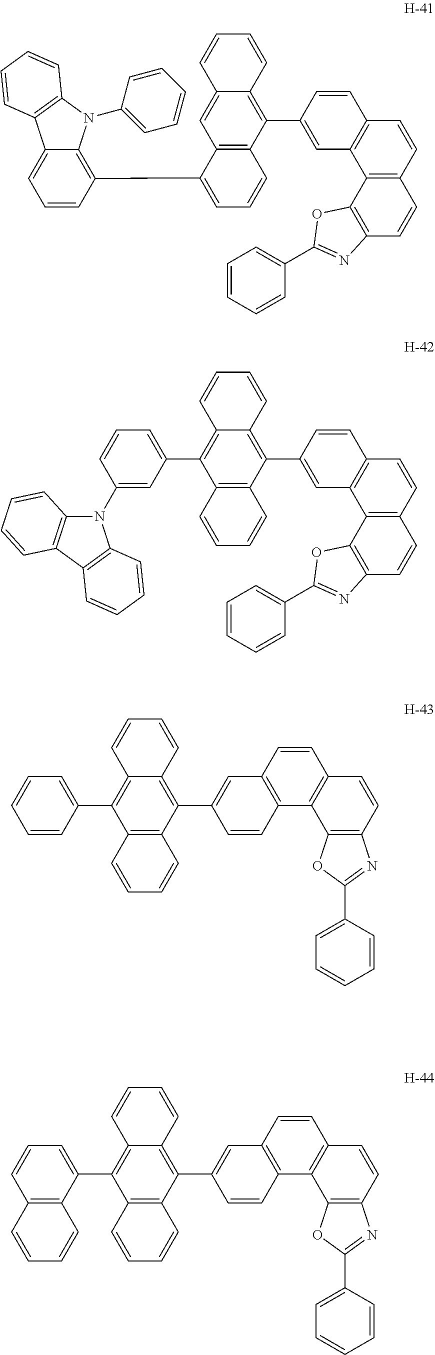 [SMILES: c1ccc(-c2nc3ccc4ccc5ccc(-c6c7ccccc7cc7c(-c8cccc9c%10ccccc%10n(-c%10ccccc%10)c89)cccc67)cc5c4c3o2)cc1 ; c1ccc(-c2nc3ccc4ccc5ccc(-c6c7ccccc7c(-c7cccc(-n8c9ccccc9c9ccccc98)c7)c7ccccc67)cc5c4c3o2)cc1 ; c1ccc(-c2nc3ccc4ccc5cc(-c6c7ccccc7c(-c7ccccc7)c7ccccc67)ccc5c4c3o2)cc1 ; c1ccc(-c2nc3ccc4ccc5cc(-c6c7ccccc7c(-c7cccc8ccccc78)c7ccccc67)ccc5c4c3o2)cc1]

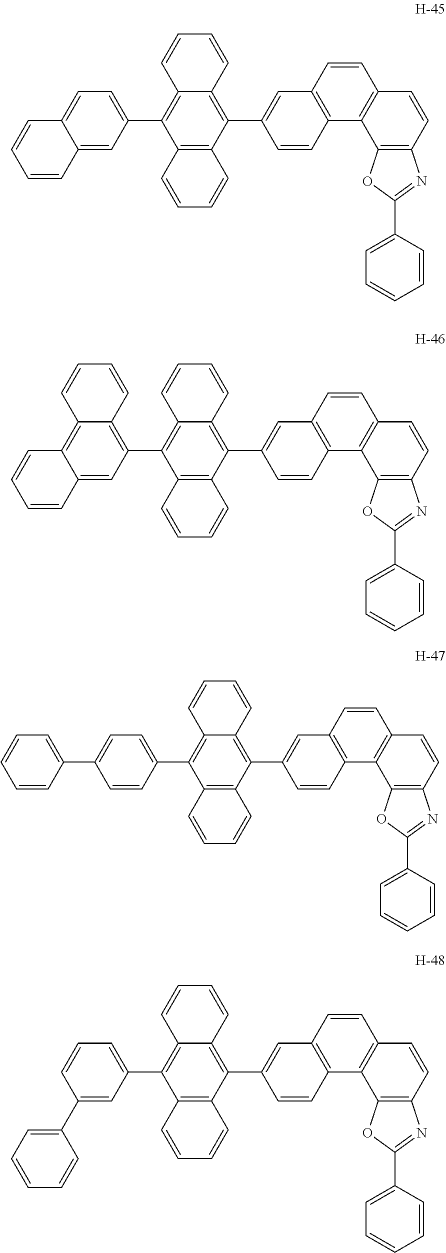 [SMILES: c1ccc(-c2nc3ccc4ccc5cc(-c6c7ccccc7c(-c7ccc8ccccc8c7)c7ccccc67)ccc5c4c3o2)cc1 ; c1ccc(-c2nc3ccc4ccc5cc(-c6c7ccccc7c(-c7cc8ccccc8c8ccccc78)c7ccccc67)ccc5c4c3o2)cc1 ; c1ccc(-c2ccc(-c3c4ccccc4c(-c4ccc5c(ccc6ccc7nc(-c8ccccc8)oc7c65)c4)c4ccccc34)cc2)cc1 ; c1ccc(-c2cccc(-c3c4ccccc4c(-c4ccc5c(ccc6ccc7nc(-c8ccccc8)oc7c65)c4)c4ccccc34)c2)cc1]

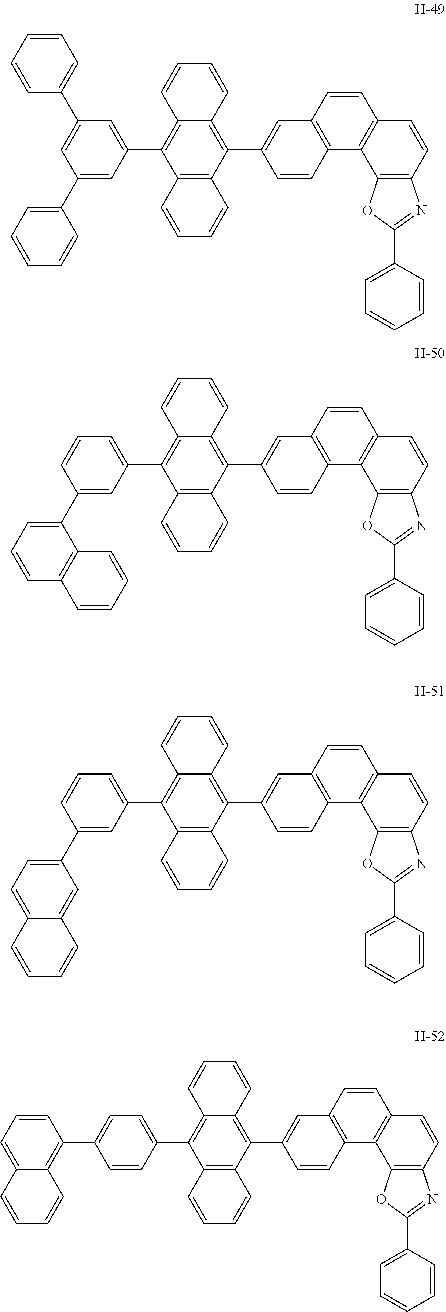 [SMILES: c1ccc(-c2cc(-c3ccccc3)cc(-c3c4ccccc4c(-c4ccc5c(ccc6ccc7nc(-c8ccccc8)oc7c65)c4)c4ccccc34)c2)cc1 ; c1ccc(-c2nc3ccc4ccc5cc(-c6c7ccccc7c(-c7cccc(-c8cccc9ccccc89)c7)c7ccccc67)ccc5c4c3o2)cc1 ; c1ccc(-c2nc3ccc4ccc5cc(-c6c7ccccc7c(-c7cccc(-c8ccc9ccccc9c8)c7)c7ccccc67)ccc5c4c3o2)cc1 ; c1ccc(-c2nc3ccc4ccc5cc(-c6c7ccccc7c(-c7ccc(-c8cccc9ccccc89)cc7)c7ccccc67)ccc5c4c3o2)cc1]

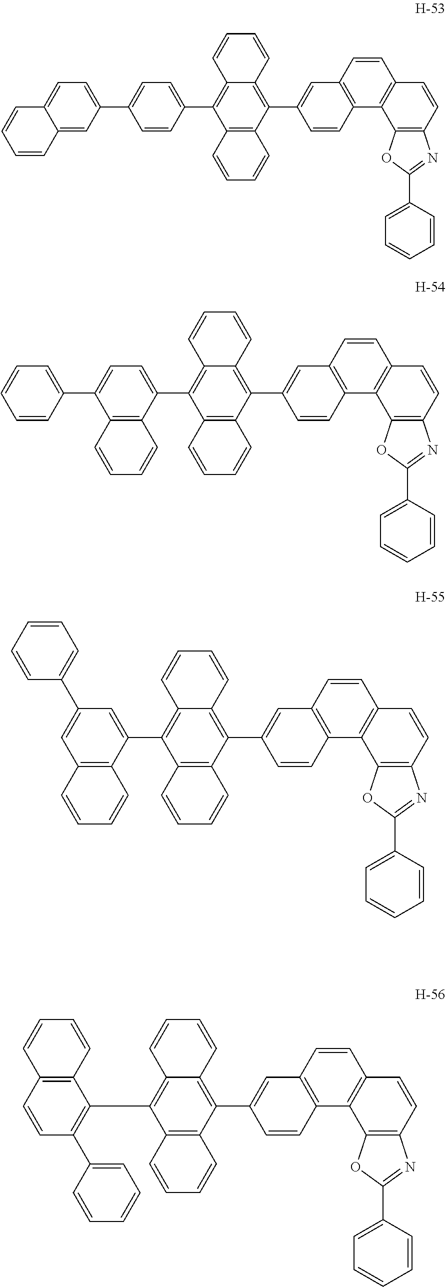 [SMILES: c1ccc(-c2nc3ccc4ccc5cc(-c6c7ccccc7c(-c7ccc(-c8ccc9ccccc9c8)cc7)c7ccccc67)ccc5c4c3o2)cc1 ; c1ccc(-c2nc3ccc4ccc5cc(-c6c7ccccc7c(-c7ccc(-c8ccccc8)c8ccccc78)c7ccccc67)ccc5c4c3o2)cc1 ; c1ccc(-c2cc(-c3c4ccccc4c(-c4ccc5c(ccc6ccc7nc(-c8ccccc8)oc7c65)c4)c4ccccc34)c3ccccc3c2)cc1 ; c1ccc(-c2nc3ccc4ccc5cc(-c6c7ccccc7c(-c7c(-c8ccccc8)ccc8ccccc78)c7ccccc67)ccc5c4c3o2)cc1]

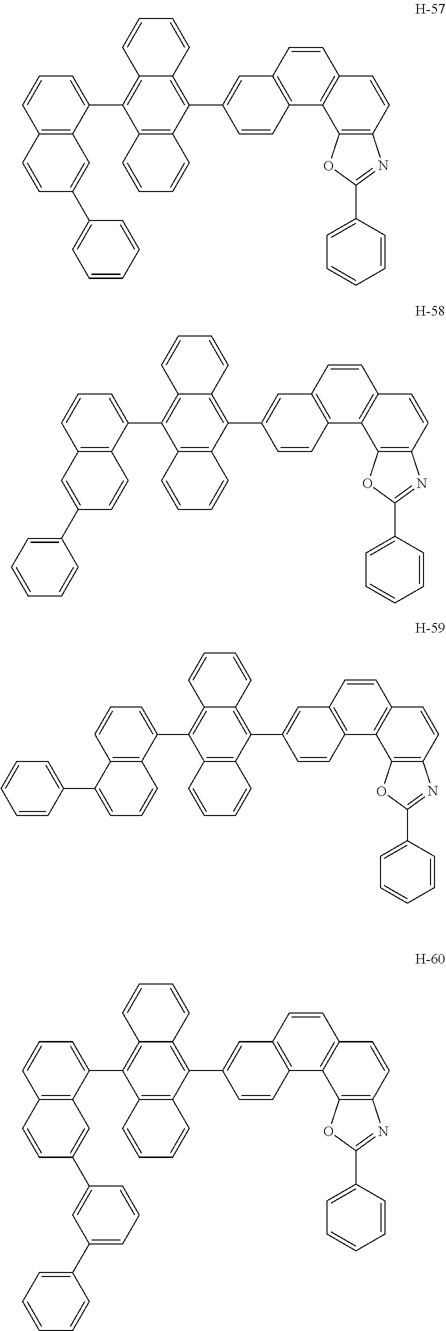 [SMILES: c1ccc(-c2ccc3cccc(-c4c5ccccc5c(-c5ccc6c(ccc7ccc8nc(-c9ccccc9)oc8c76)c5)c5ccccc45)c3c2)cc1 ; c1ccc(-c2ccc3c(-c4c5ccccc5c(-c5ccc6c(ccc7ccc8nc(-c9ccccc9)oc8c76)c5)c5ccccc45)cccc3c2)cc1 ; c1ccc(-c2nc3ccc4ccc5cc(-c6c7ccccc7c(-c7cccc8c(-c9ccccc9)cccc78)c7ccccc67)ccc5c4c3o2)cc1 ; c1ccc(-c2cccc(-c3ccc4cccc(-c5c6ccccc6c(-c6ccc7c(ccc8ccc9nc(-c%10ccccc%10)oc9c87)c6)c6ccccc56)c4c3)c2)cc1]

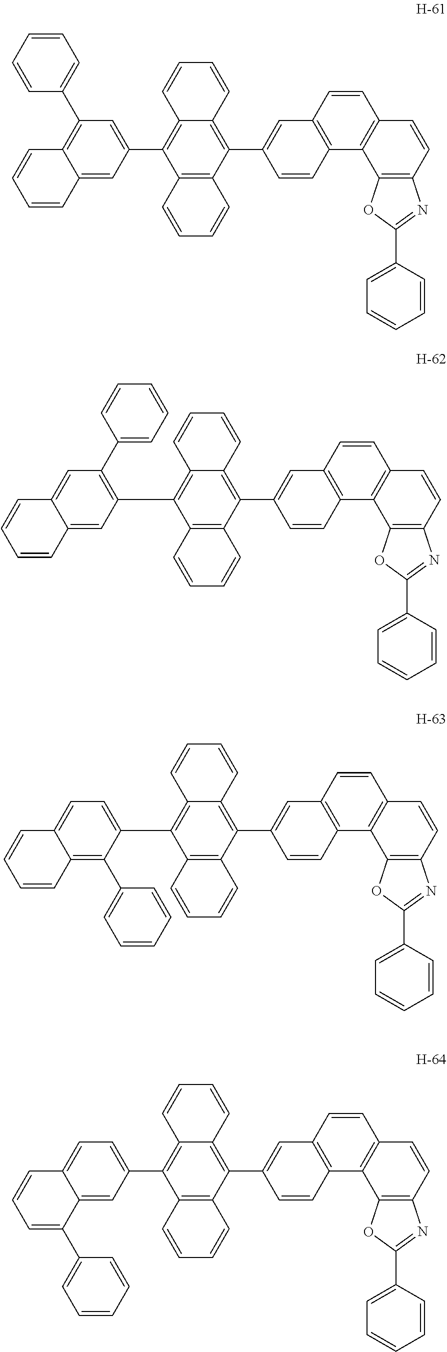 [SMILES: c1ccc(-c2nc3ccc4ccc5cc(-c6c7ccccc7c(-c7cc(-c8ccccc8)c8ccccc8c7)c7ccccc67)ccc5c4c3o2)cc1 ; c1ccc(-c2nc3ccc4ccc5cc(-c6c7ccccc7c(-c7cc8ccccc8cc7-c7ccccc7)c7ccccc67)ccc5c4c3o2)cc1 ; c1ccc(-c2nc3ccc4ccc5cc(-c6c7ccccc7c(-c7ccc8ccccc8c7-c7ccccc7)c7ccccc67)ccc5c4c3o2)cc1 ; c1ccc(-c2nc3ccc4ccc5cc(-c6c7ccccc7c(-c7ccc8cccc(-c9ccccc9)c8c7)c7ccccc67)ccc5c4c3o2)cc1]

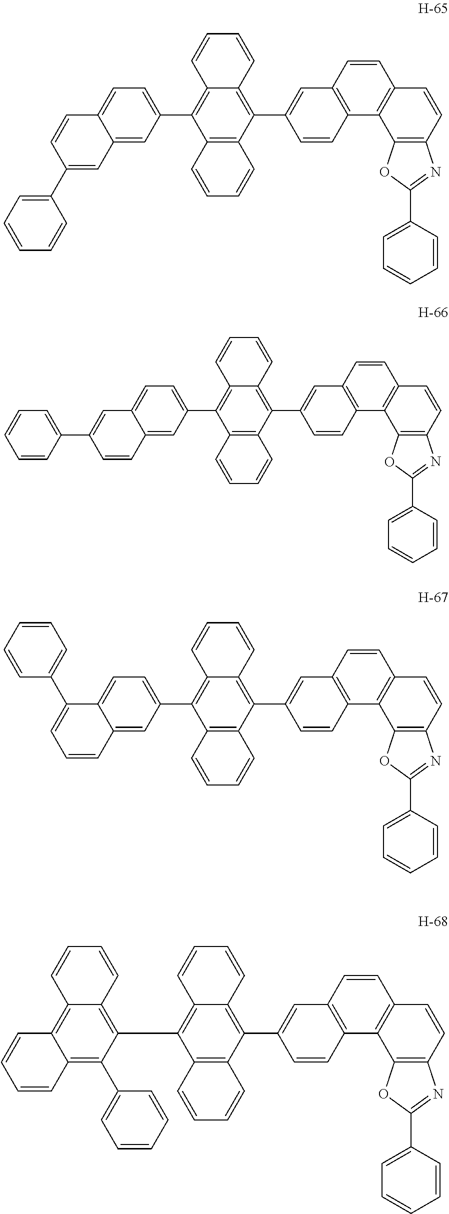 [SMILES: c1ccc(-c2ccc3ccc(-c4c5ccccc5c(-c5ccc6c(ccc7ccc8nc(-c9ccccc9)oc8c76)c5)c5ccccc45)cc3c2)cc1 ; c1ccc(-c2ccc3cc(-c4c5ccccc5c(-c5ccc6c(ccc7ccc8nc(-c9ccccc9)oc8c76)c5)c5ccccc45)ccc3c2)cc1 ; c1ccc(-c2nc3ccc4ccc5cc(-c6c7ccccc7c(-c7ccc8c(-c9ccccc9)cccc8c7)c7ccccc67)ccc5c4c3o2)cc1 ; c1ccc(-c2nc3ccc4ccc5cc(-c6c7ccccc7c(-c7c(-c8ccccc8)c8ccccc8c8ccccc78)c7ccccc67)ccc5c4c3o2)cc1]

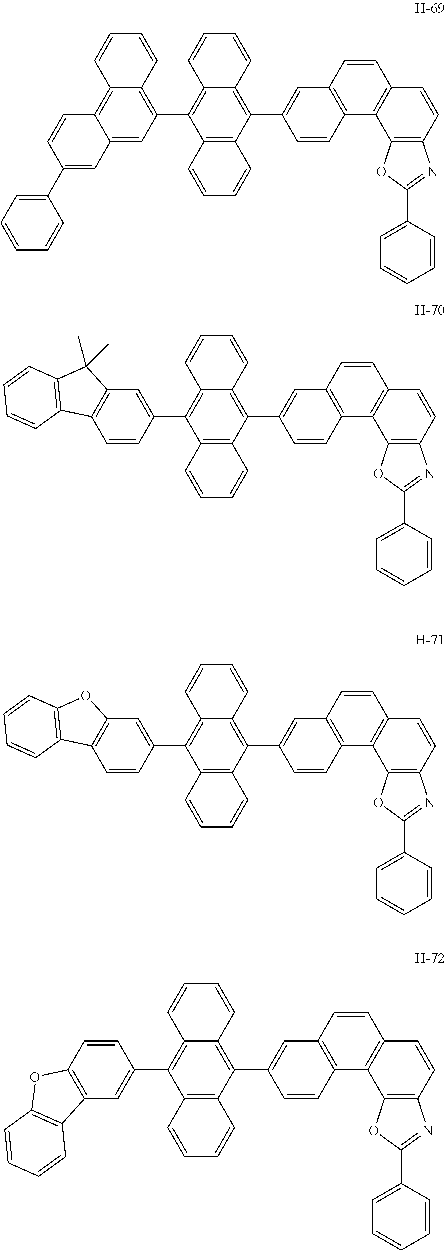 [SMILES: c1ccc(-c2ccc3c(c2)cc(-c2c4ccccc4c(-c4ccc5c(ccc6ccc7nc(-c8ccccc8)oc7c65)c4)c4ccccc24)c2ccccc23)cc1 ; CC1(C)c2ccccc2-c2ccc(-c3c4ccccc4c(-c4ccc5c(ccc6ccc7nc(-c8ccccc8)oc7c65)c4)c4ccccc34)cc21 ; c1ccc(-c2nc3ccc4ccc5cc(-c6c7ccccc7c(-c7ccc8c(c7)oc7ccccc78)c7ccccc67)ccc5c4c3o2)cc1 ; c1ccc(-c2nc3ccc4ccc5cc(-c6c7ccccc7c(-c7ccc8oc9ccccc9c8c7)c7ccccc67)ccc5c4c3o2)cc1]

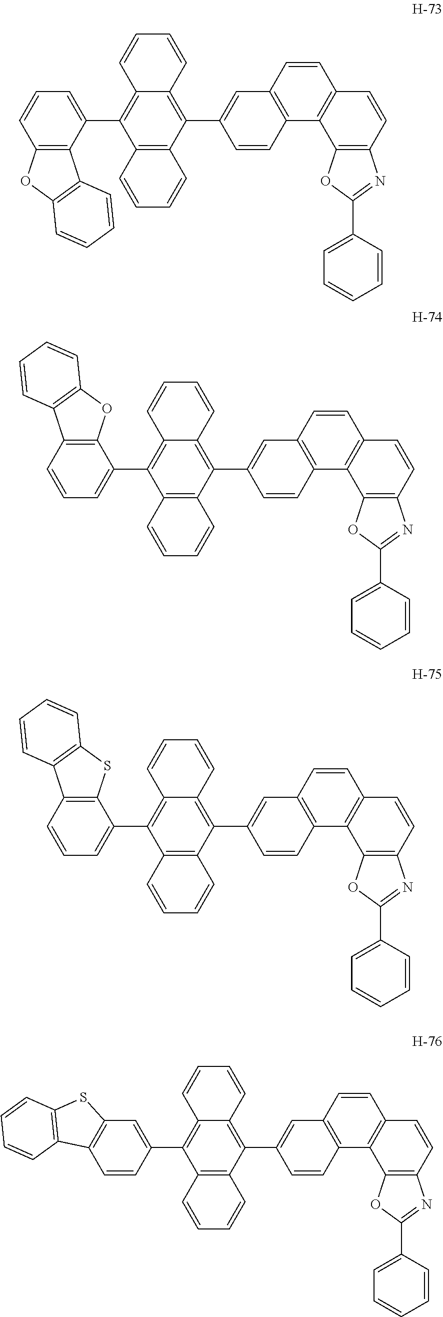 [SMILES: c1ccc(-c2nc3ccc4ccc5cc(-c6c7ccccc7c(-c7cccc8oc9ccccc9c78)c7ccccc67)ccc5c4c3o2)cc1 ; c1ccc(-c2nc3ccc4ccc5cc(-c6c7ccccc7c(-c7cccc8c7oc7ccccc78)c7ccccc67)ccc5c4c3o2)cc1 ; c1ccc(-c2nc3ccc4ccc5cc(-c6c7ccccc7c(-c7cccc8c7sc7ccccc78)c7ccccc67)ccc5c4c3o2)cc1 ; c1ccc(-c2nc3ccc4ccc5cc(-c6c7ccccc7c(-c7ccc8c(c7)sc7ccccc78)c7ccccc67)ccc5c4c3o2)cc1]

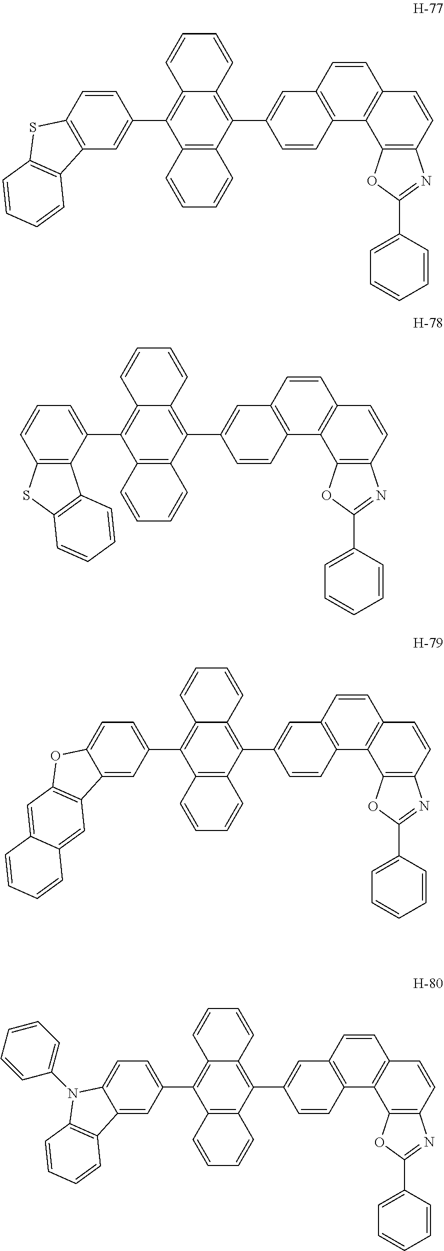 [SMILES: c1ccc(-c2nc3ccc4ccc5cc(-c6c7ccccc7c(-c7ccc8sc9ccccc9c8c7)c7ccccc67)ccc5c4c3o2)cc1 ; c1ccc(-c2nc3ccc4ccc5cc(-c6c7ccccc7c(-c7cccc8sc9ccccc9c78)c7ccccc67)ccc5c4c3o2)cc1 ; c1ccc(-c2nc3ccc4ccc5cc(-c6c7ccccc7c(-c7ccc8oc9cc%10ccccc%10cc9c8c7)c7ccccc67)ccc5c4c3o2)cc1 ; c1ccc(-c2nc3ccc4ccc5cc(-c6c7ccccc7c(-c7ccc8c(c7)c7ccccc7n8-c7ccccc7)c7ccccc67)ccc5c4c3o2)cc1]

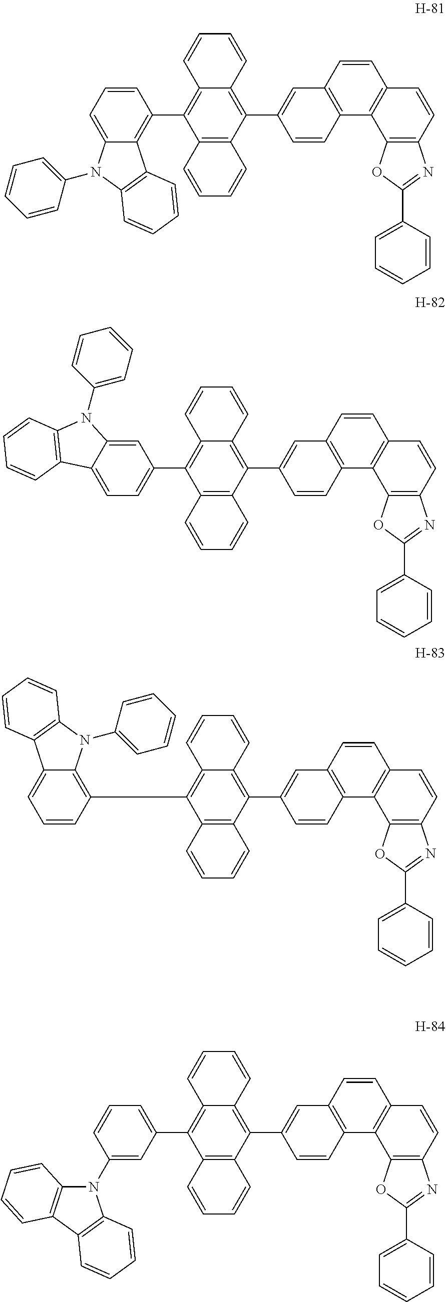 [SMILES: c1ccc(-c2nc3ccc4ccc5cc(-c6c7ccccc7c(-c7cccc8c7c7ccccc7n8-c7ccccc7)c7ccccc67)ccc5c4c3o2)cc1 ; c1ccc(-c2nc3ccc4ccc5cc(-c6c7ccccc7c(-c7ccc8c9ccccc9n(-c9ccccc9)c8c7)c7ccccc67)ccc5c4c3o2)cc1 ; c1ccc(-c2nc3ccc4ccc5cc(-c6c7ccccc7c(-c7cccc8c9ccccc9n(-c9ccccc9)c78)c7ccccc67)ccc5c4c3o2)cc1 ; c1ccc(-c2nc3ccc4ccc5cc(-c6c7ccccc7c(-c7cccc(-n8c9ccccc9c9ccccc98)c7)c7ccccc67)ccc5c4c3o2)cc1]

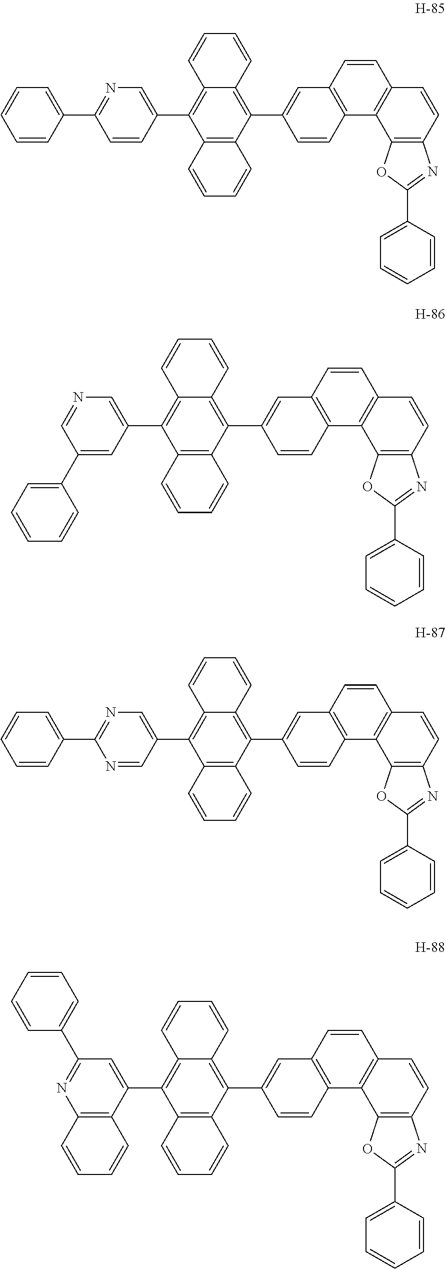 [SMILES: c1ccc(-c2ccc(-c3c4ccccc4c(-c4ccc5c(ccc6ccc7nc(-c8ccccc8)oc7c65)c4)c4ccccc34)cn2)cc1 ; c1ccc(-c2cncc(-c3c4ccccc4c(-c4ccc5c(ccc6ccc7nc(-c8ccccc8)oc7c65)c4)c4ccccc34)c2)cc1 ; c1ccc(-c2ncc(-c3c4ccccc4c(-c4ccc5c(ccc6ccc7nc(-c8ccccc8)oc7c65)c4)c4ccccc34)cn2)cc1 ; c1ccc(-c2cc(-c3c4ccccc4c(-c4ccc5c(ccc6ccc7nc(-c8ccccc8)oc7c65)c4)c4ccccc34)c3ccccc3n2)cc1]

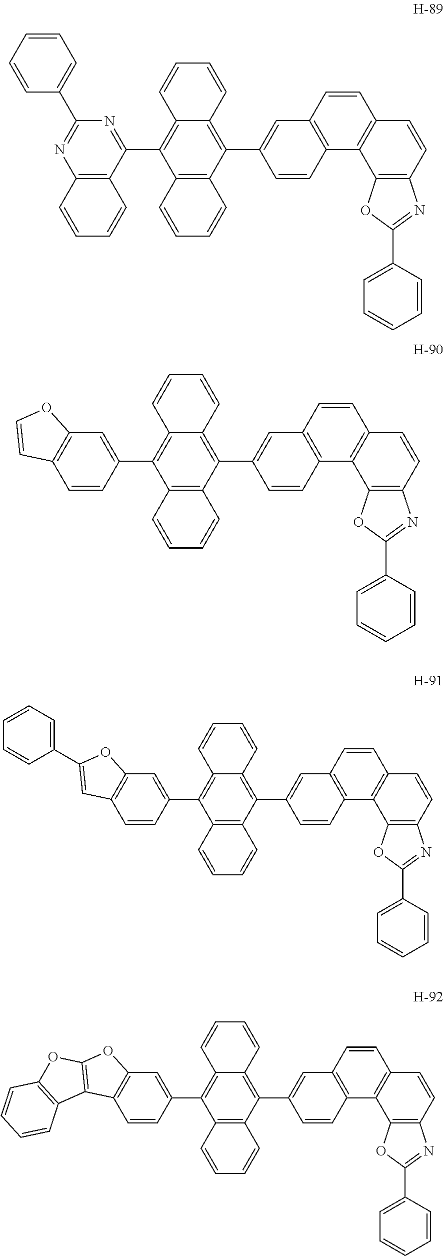 [SMILES: c1ccc(-c2nc(-c3c4ccccc4c(-c4ccc5c(ccc6ccc7nc(-c8ccccc8)oc7c65)c4)c4ccccc34)c3ccccc3n2)cc1 ; c1ccc(-c2nc3ccc4ccc5cc(-c6c7ccccc7c(-c7ccc8ccoc8c7)c7ccccc67)ccc5c4c3o2)cc1 ; c1ccc(-c2cc3ccc(-c4c5ccccc5c(-c5ccc6c(ccc7ccc8nc(-c9ccccc9)oc8c76)c5)c5ccccc45)cc3o2)cc1 ; c1ccc(-c2nc3ccc4ccc5cc(-c6c7ccccc7c(-c7ccc8c(c7)oc7oc9ccccc9c78)c7ccccc67)ccc5c4c3o2)cc1]

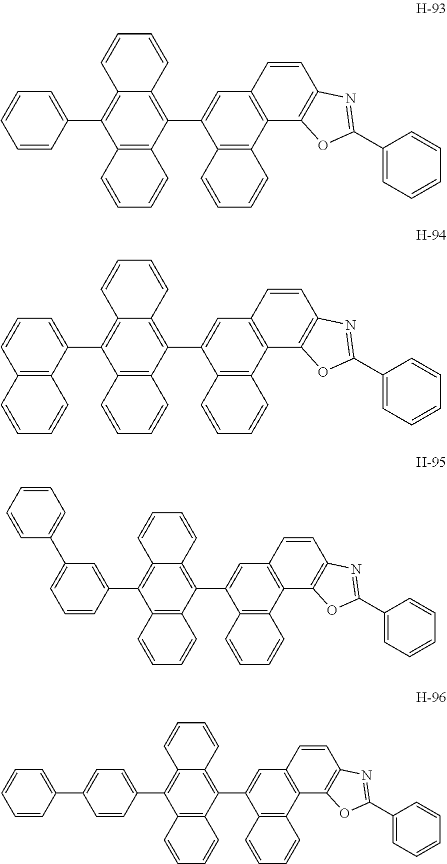 [SMILES: c1ccc(-c2nc3ccc4cc(-c5c6ccccc6c(-c6ccccc6)c6ccccc56)c5ccccc5c4c3o2)cc1 ; c1ccc(-c2nc3ccc4cc(-c5c6ccccc6c(-c6cccc7ccccc67)c6ccccc56)c5ccccc5c4c3o2)cc1 ; c1ccc(-c2cccc(-c3c4ccccc4c(-c4cc5ccc6nc(-c7ccccc7)oc6c5c5ccccc45)c4ccccc34)c2)cc1 ; c1ccc(-c2ccc(-c3c4ccccc4c(-c4cc5ccc6nc(-c7ccccc7)oc6c5c5ccccc45)c4ccccc34)cc2)cc1]

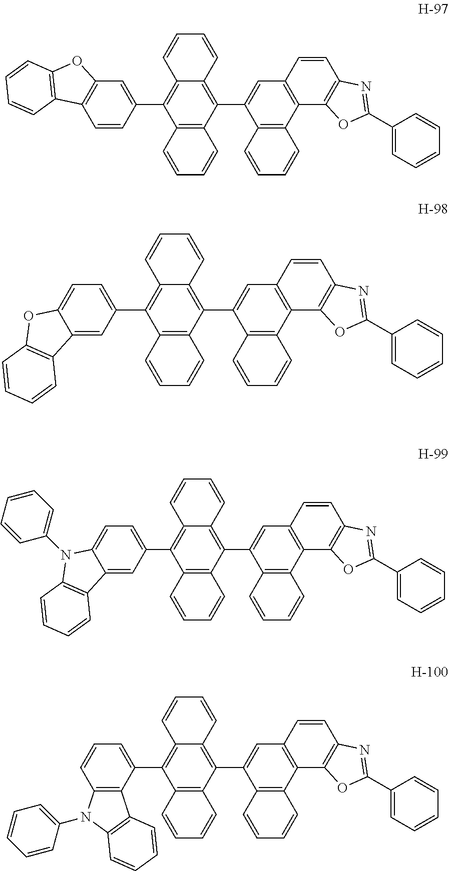 [SMILES: c1ccc(-c2nc3ccc4cc(-c5c6ccccc6c(-c6ccc7c(c6)oc6ccccc67)c6ccccc56)c5ccccc5c4c3o2)cc1 ; c1ccc(-c2nc3ccc4cc(-c5c6ccccc6c(-c6ccc7oc8ccccc8c7c6)c6ccccc56)c5ccccc5c4c3o2)cc1 ; c1ccc(-c2nc3ccc4cc(-c5c6ccccc6c(-c6ccc7c(c6)c6ccccc6n7-c6ccccc6)c6ccccc56)c5ccccc5c4c3o2)cc1 ; c1ccc(-c2nc3ccc4cc(-c5c6ccccc6c(-c6cccc7c6c6ccccc6n7-c6ccccc6)c6ccccc56)c5ccccc5c4c3o2)cc1]

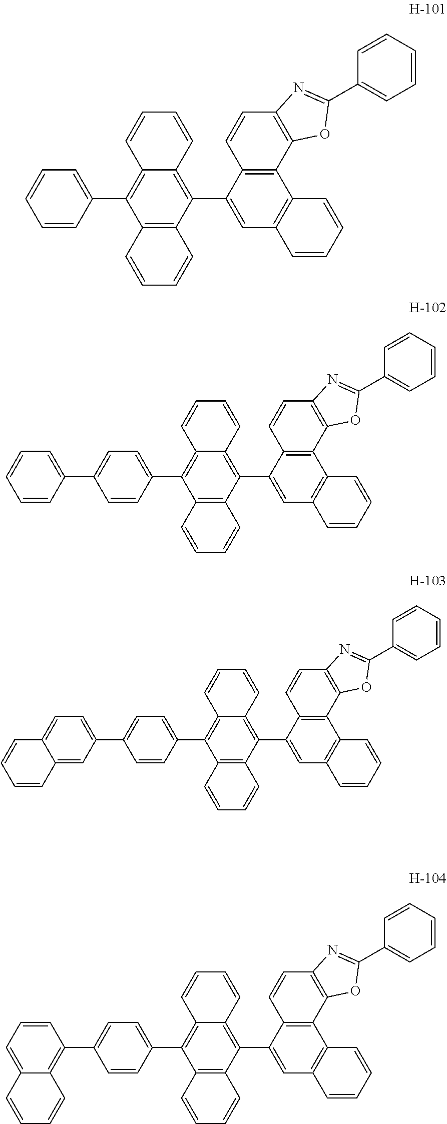 [SMILES: c1ccc(-c2nc3ccc4c(-c5c6ccccc6c(-c6ccccc6)c6ccccc56)cc5ccccc5c4c3o2)cc1 ; c1ccc(-c2ccc(-c3c4ccccc4c(-c4cc5ccccc5c5c4ccc4nc(-c6ccccc6)oc45)c4ccccc34)cc2)cc1 ; c1ccc(-c2nc3ccc4c(-c5c6ccccc6c(-c6ccc(-c7ccc8ccccc8c7)cc6)c6ccccc56)cc5ccccc5c4c3o2)cc1 ; c1ccc(-c2nc3ccc4c(-c5c6ccccc6c(-c6ccc(-c7cccc8ccccc78)cc6)c6ccccc56)cc5ccccc5c4c3o2)cc1]

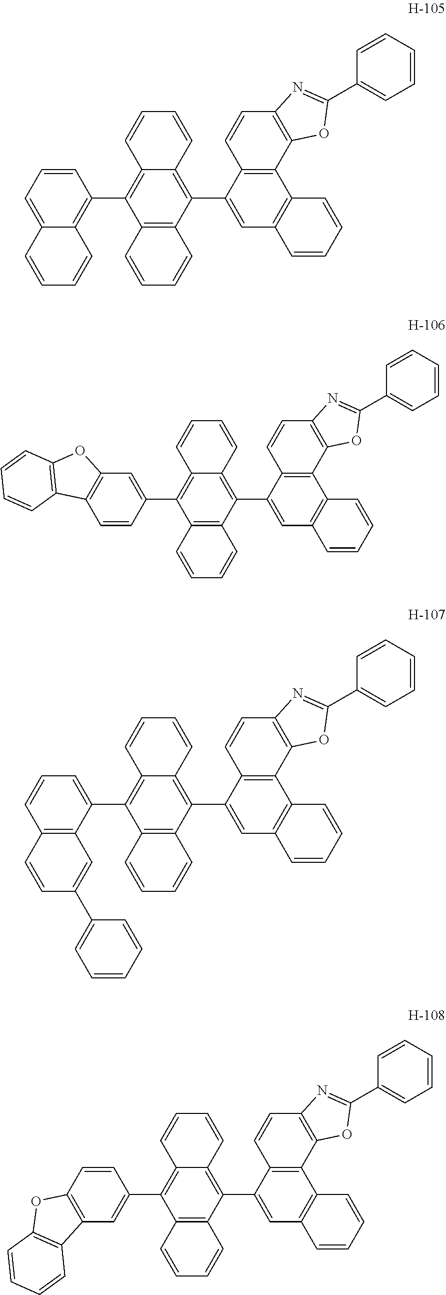 [SMILES: c1ccc(-c2nc3ccc4c(-c5c6ccccc6c(-c6cccc7ccccc67)c6ccccc56)cc5ccccc5c4c3o2)cc1 ; c1ccc(-c2nc3ccc4c(-c5c6ccccc6c(-c6ccc7c(c6)oc6ccccc67)c6ccccc56)cc5ccccc5c4c3o2)cc1 ; c1ccc(-c2ccc3cccc(-c4c5ccccc5c(-c5cc6ccccc6c6c5ccc5nc(-c7ccccc7)oc56)c5ccccc45)c3c2)cc1 ; c1ccc(-c2nc3ccc4c(-c5c6ccccc6c(-c6ccc7oc8ccccc8c7c6)c6ccccc56)cc5ccccc5c4c3o2)cc1]

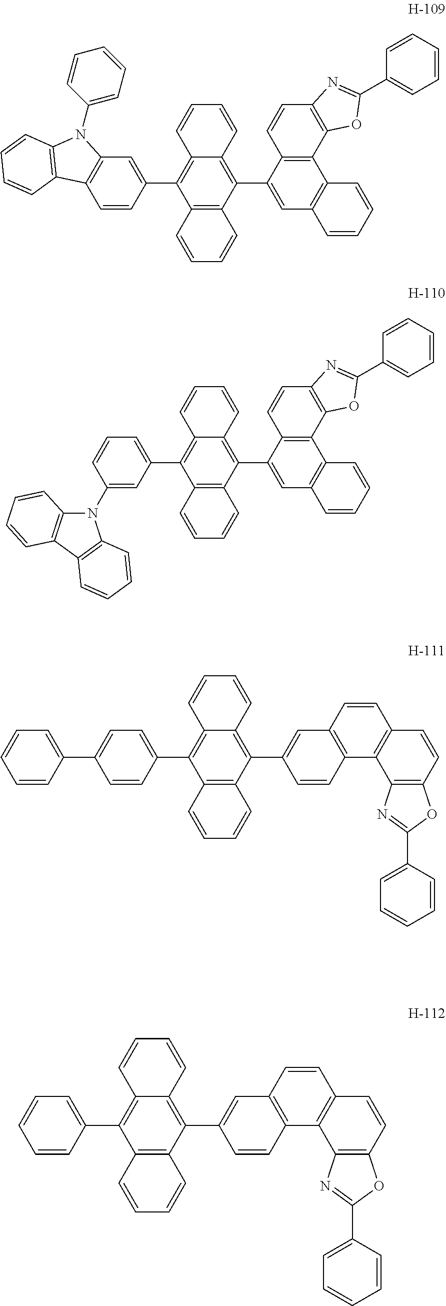 [SMILES: c1ccc(-c2nc3ccc4c(-c5c6ccccc6c(-c6ccc7c8ccccc8n(-c8ccccc8)c7c6)c6ccccc56)cc5ccccc5c4c3o2)cc1 ; c1ccc(-c2nc3ccc4c(-c5c6ccccc6c(-c6cccc(-n7c8ccccc8c8ccccc87)c6)c6ccccc56)cc5ccccc5c4c3o2)cc1 ; c1ccc(-c2ccc(-c3c4ccccc4c(-c4ccc5c(ccc6ccc7oc(-c8ccccc8)nc7c65)c4)c4ccccc34)cc2)cc1 ; c1ccc(-c2nc3c(ccc4ccc5cc(-c6c7ccccc7c(-c7ccccc7)c7ccccc67)ccc5c43)o2)cc1]

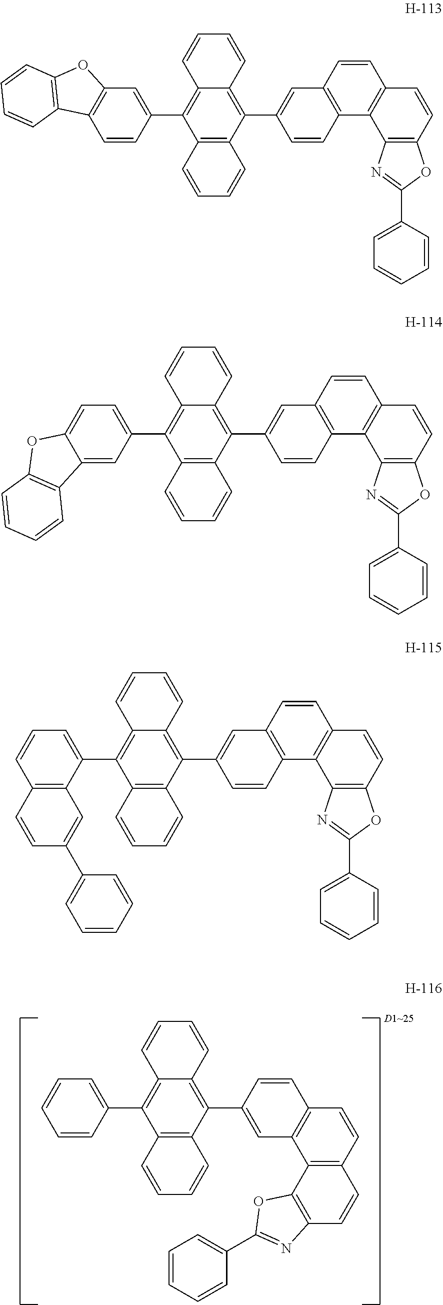

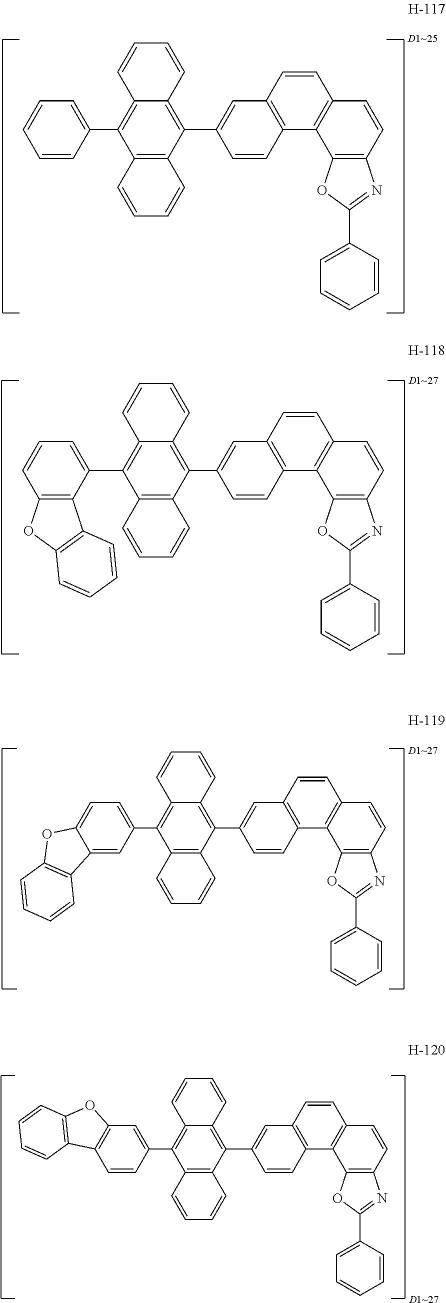

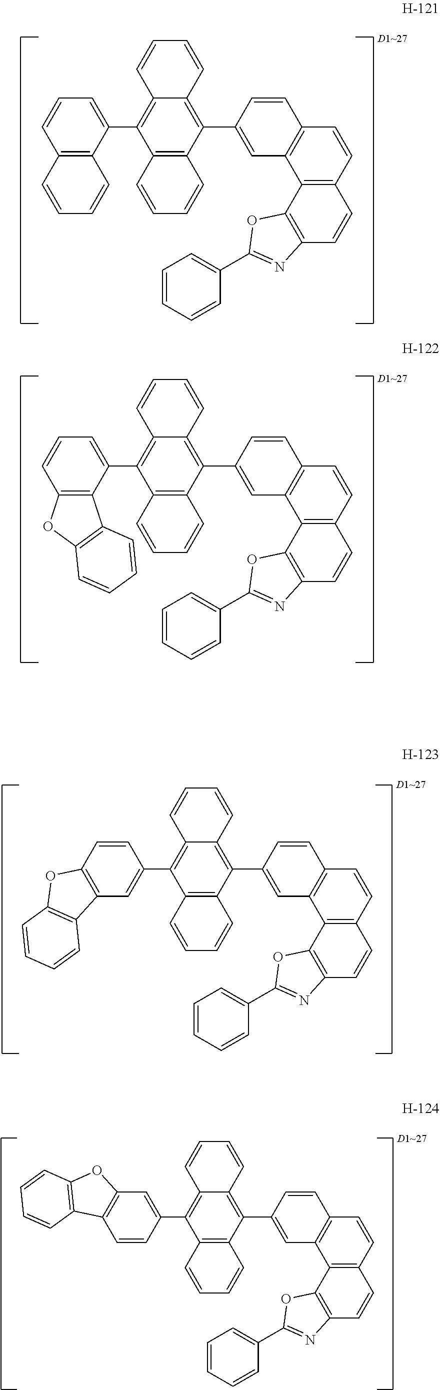

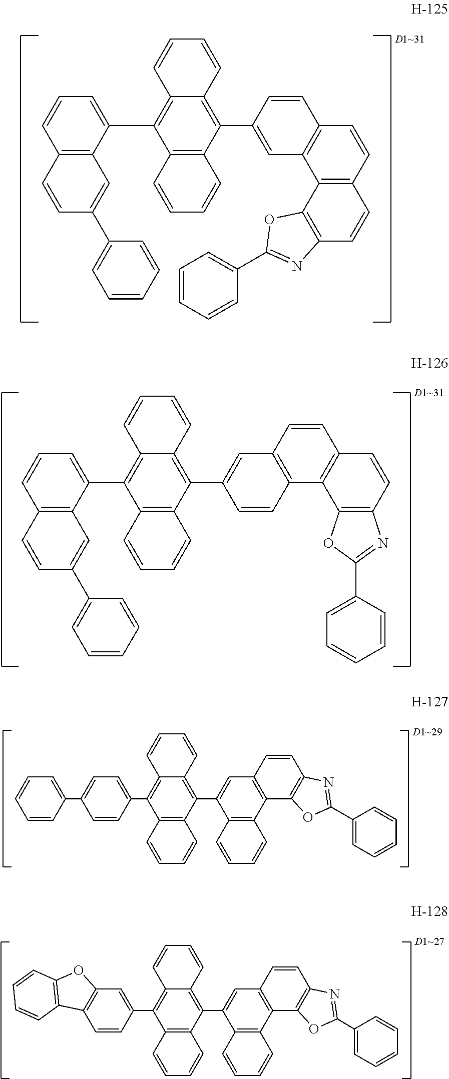

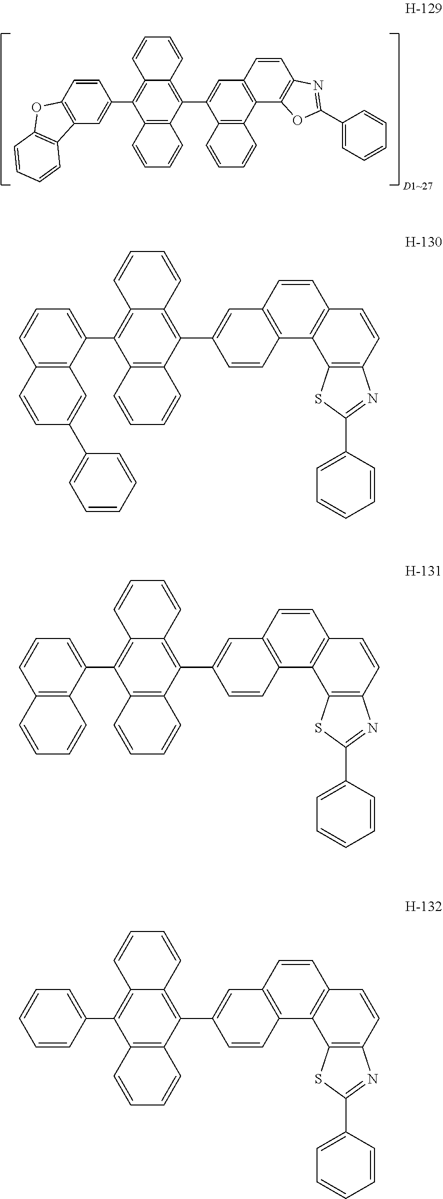

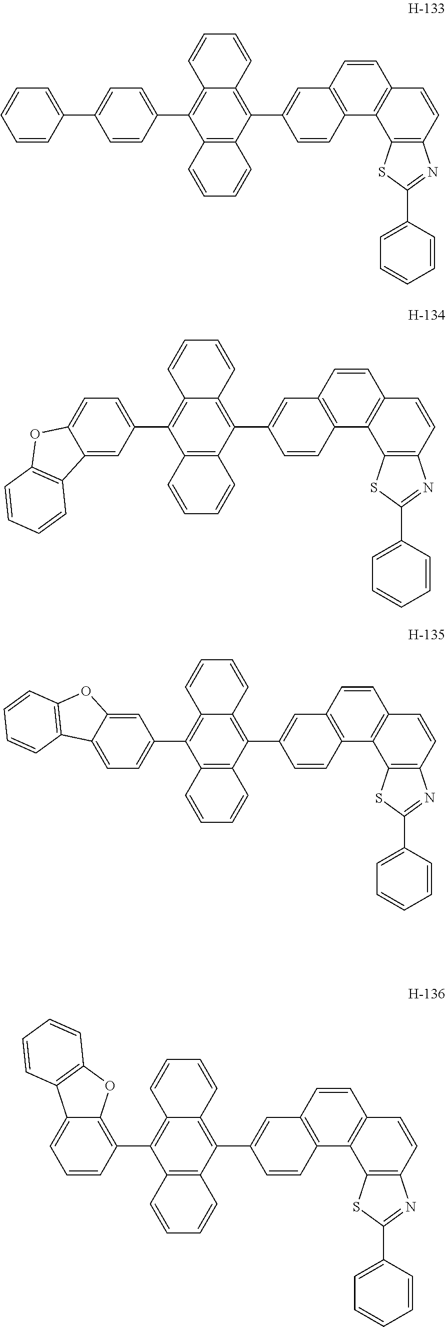 [SMILES: c1ccc(-c2ccc(-c3c4ccccc4c(-c4ccc5c(ccc6ccc7nc(-c8ccccc8)sc7c65)c4)c4ccccc34)cc2)cc1 ; c1ccc(-c2nc3ccc4ccc5cc(-c6c7ccccc7c(-c7ccc8oc9ccccc9c8c7)c7ccccc67)ccc5c4c3s2)cc1 ; c1ccc(-c2nc3ccc4ccc5cc(-c6c7ccccc7c(-c7ccc8c(c7)oc7ccccc78)c7ccccc67)ccc5c4c3s2)cc1 ; c1ccc(-c2nc3ccc4ccc5cc(-c6c7ccccc7c(-c7cccc8c7oc7ccccc78)c7ccccc67)ccc5c4c3s2)cc1]

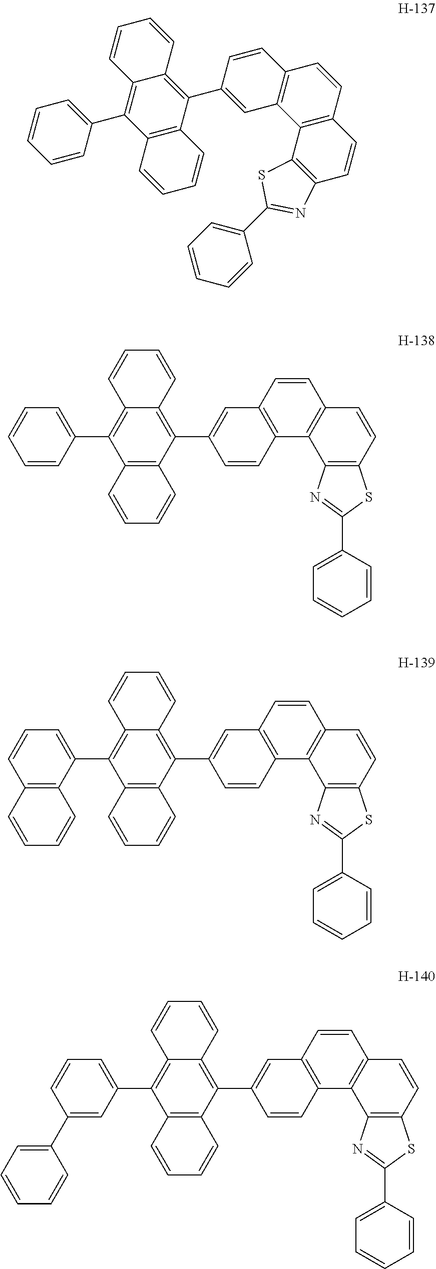 [SMILES: c1ccc(-c2nc3ccc4ccc5ccc(-c6c7ccccc7c(-c7ccccc7)c7ccccc67)cc5c4c3s2)cc1 ; c1ccc(-c2nc3c(ccc4ccc5cc(-c6c7ccccc7c(-c7ccccc7)c7ccccc67)ccc5c43)s2)cc1 ; c1ccc(-c2nc3c(ccc4ccc5cc(-c6c7ccccc7c(-c7cccc8ccccc78)c7ccccc67)ccc5c43)s2)cc1 ; c1ccc(-c2cccc(-c3c4ccccc4c(-c4ccc5c(ccc6ccc7sc(-c8ccccc8)nc7c65)c4)c4ccccc34)c2)cc1]

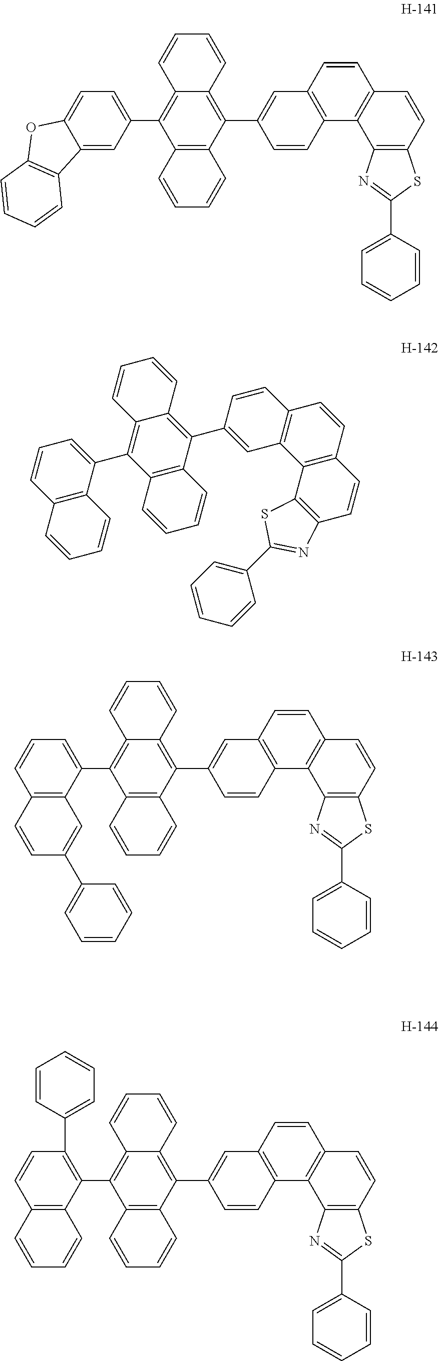 [SMILES: c1ccc(-c2nc3c(ccc4ccc5cc(-c6c7ccccc7c(-c7ccc8oc9ccccc9c8c7)c7ccccc67)ccc5c43)s2)cc1 ; c1ccc(-c2nc3ccc4ccc5ccc(-c6c7ccccc7c(-c7cccc8ccccc78)c7ccccc67)cc5c4c3s2)cc1 ; c1ccc(-c2ccc3cccc(-c4c5ccccc5c(-c5ccc6c(ccc7ccc8sc(-c9ccccc9)nc8c76)c5)c5ccccc45)c3c2)cc1 ; c1ccc(-c2nc3c(ccc4ccc5cc(-c6c7ccccc7c(-c7c(-c8ccccc8)ccc8ccccc78)c7ccccc67)ccc5c43)s2)cc1]

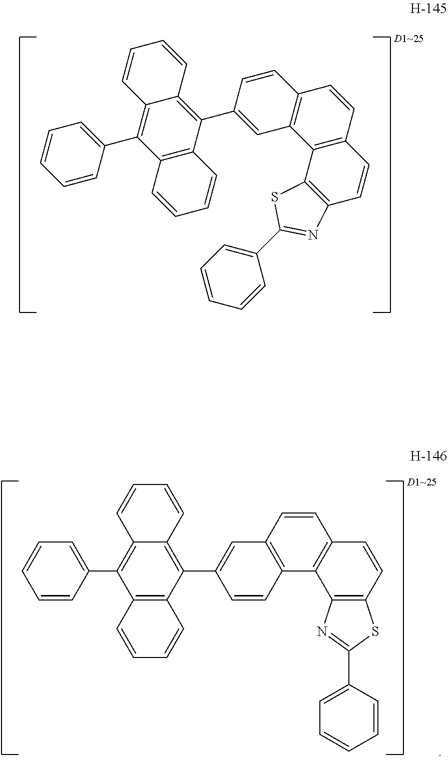

5. An organic electroluminescent material comprising the organic electroluminescent compound according to claim 1.

6. An organic electroluminescent device comprising the organic electroluminescent compound according to claim 1.

7. The organic electroluminescent device according to claim 6, wherein the organic electroluminescent compound is comprised as a host material.
